SEQUENCE LISTING (59 sequences)

SEQ ID NO: 1 - protein, 199 aa, Homo sapiens
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 2 - protein, 918 aa, Homo sapiens
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 3 - protein, 422 aa, Homo sapiens
MSSSCSGLSRVLVAVATALVSASSPCPQAWGPPGVQYGQPGRSVKLCCPGVTAGDPVSWFRDGEPKLLQGPDSGLGHELVLAQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGTIPKEIPAWGQLHTQPEVEPQVDSPAPPRPSLQPHPRLLDHRDSVEQVAVLASLGILSFLGLVAGALALGLWLRLRRGGKDGSPKPGFLASVIPVDRRPGAPNL

SEQ ID NO: 4 - DNA, 19 bp, Artificial Sequence
  synthetic construct
ccttccaaagccagatctt

SEQ ID NO: 5 - DNA, 19 bp, Artificial Sequence
  synthetic construct
gcctgggcaggaacatata

SEQ ID NO: 6 - DNA, 19 bp, Artificial Sequence
  synthetic construct
cctgggcaggaacatatat

SEQ ID NO: 7 - DNA, 19 bp, Artificial Sequence
  synthetic construct
ggttcattatggctgtgtt

SEQ ID NO: 8 - DNA, 19 bp, Artificial Sequence
  synthetic construct
ggaccataccaaaggagat

SEQ ID NO: 9 - DNA, 19 bp, Artificial Sequence
  synthetic construct
gcgtctttgggaatccttt

SEQ ID NO: 10 - DNA, 18 bp, Artificial Sequence
  synthetic construct
gcaggacagtagatccct

SEQ ID NO: 11 - DNA, 19 bp, Artificial Sequence
  synthetic construct
gctcaaggaacgtgtgtaa

SEQ ID NO: 12 - RNA, 42 nt, Artificial Sequence
  synthetic construct
  misc_feature (20)..(21): n = deoxythmidine
  misc_feature (41)..(42): n = deoxythmidine
ccuuccaaagccagaucuunnaagaucuggcuuuggaaggnn

SEQ ID NO: 13 - RNA, 42 nt, Artificial Sequence
  synthetic construct
  misc_feature (20)..(21): n = deoxythmidine
  misc_feature (41)..(42): n = deoxythmidine
gccugggcaggaacauauannuauauguuccugcccaggcnn

SEQ ID NO: 14 - RNA, 42 nt, Artificial Sequence
  synthetic construct
  misc_feature (20)..(21): n = deoxythmidine
  misc_feature (41)..(42): n = deoxythmidine
ccugggcaggaacauauaunnauauauguuccugcccaggnn

SEQ ID NO: 15 - RNA, 42 nt, Artificial Sequence
  synthetic construct
  misc_feature (20)..(21): n = deoxythmidine
  misc_feature (41)..(42): n = deoxythmidine
gguucauuauggcuguguunnaacacagccauaaugaaccnn

SEQ ID NO: 16 - RNA, 42 nt, Artificial Sequence
  synthetic construct
  misc_feature (20)..(21): n = deoxythmidine
  misc_feature (41)..(42): n = deoxythmidine
ggaccauaccaaaggagaunnaucuccuuugguaugguccnn

SEQ ID NO: 17 - RNA, 42 nt, Artificial Sequence
  synthetic construct
  misc_feature (20)..(21): n = deoxythmidine
  misc_feature (41)..(42): n = deoxythmidine
gcgucuuugggaauccuuunnaaaggauucccaaagacgcnn

SEQ ID NO: 18 - RNA, 42 nt, Artificial Sequence
  synthetic construct
  misc_feature (20)..(21): n = deoxythmidine
  misc_feature (41)..(42): n = deoxythmidine
gcaggacaguagaucccuannuagggaucuacuguccugcnn

SEQ ID NO: 19 - RNA, 42 nt, Artificial Sequence
  synthetic construct
  misc_feature (20)..(21): n = deoxythmidine
  misc_feature (41)..(42): n = deoxythmidine
gcucaaggaacguguguaannuuacacacguuccuugagcnn

SEQ ID NO: 20 - protein, 21 aa, Artificial Sequence
  synthetic construct
GPAGQSGGGGGSGGGSGGGSV

SEQ ID NO: 21 - protein, 516 aa, Artificial Sequence
  synthetic construct
MSSSCSGLSRVLVAVATALVSASSPCPQAWGPPGVQYGQPGRSVKLCCPGVTAGDPVSWFRDGEPKLLQGPDSGLGHELVLAQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGPAGQSGGGGGSGGGSGGGSVPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 22 - protein, 119 aa, Artificial Sequence
  synthetic construct
EVQLQQSGPELVKPGASVKIPCKASGYTFTDYNMDWVKQSHGKSLEWIGDINPHNGGPIYNQKFTGKATLTVDKSSSTAYMELRSLTSEDTAVYYCARGELGHWYFDVWGTGTTVTVSS

SEQ ID NO: 23 - protein, 111 aa, Artificial Sequence
  synthetic construct
DIVLTQSPASLAVSLGQRATISCRASKSVSTSGYSYIHWYQQKPGQPPKLLIYLASNLDSGVPARFSGSGSGTDFTLNIHPVEEEDAATYYCQHSRDLPPTFGGGTKLEIK

SEQ ID NO: 24 - protein, 117 aa, Artificial Sequence
  synthetic construct
QVQLQQPGAELVRPGSSVKLSCKASGYTFTNYWMHWLKQRPVQGLEWIGNIGPSDSKTHYNQKFKDKATLTVDKSSSTAYMQLNSLTSEDSAVYYCARGDYVLFTYWGQGTLVTVSA

SEQ ID NO: 25 - protein, 107 aa, Artificial Sequence
  synthetic construct
DIVLTQSPATLSLSPGERATLSCRASQSISNNLHWYQQKSHEAPRLLIKYASQSISGIPARFSGSGSGTDFTLSFSSLETEDFAVYFCQQSYSWPLTFGQGTKLEIK

SEQ ID NO: 26 - protein, 118 aa, Artificial Sequence
  synthetic construct
QVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAKIGATDPLDYWGQGTLVTVSS

SEQ ID NO: 27 - protein, 111 aa, Artificial Sequence
  synthetic construct
QSALTQPRSVSGSPGQSVTLSCTGTSSDVGGYNYVSWYQHYPGKAPKLMIFDVNERSSGVPDRFSGSKSGNTASLTISGLQAEDEADYYCASYAGRYTWMFGGGTKVTVLG

SEQ ID NO: 28 - protein, 445 aa, Artificial Sequence
  synthetic construct
QVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAKIGATDPLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 216 aa, Artificial Sequence
  synthetic construct
QSALTQPRSVSGSPGQSVTLSCTGTSSDVGGYNYVSWYQHYPGKAPKLMIFDVNERSSGVPDRFSGSKSGNTASLTISGLQAEDEADYYCASYAGRYTWMFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 30 - protein, 119 aa, Artificial Sequence
  synthetic construct
EVQLVQSGAEVKKPGASVKISCKASGYTFTDYNMDWVKQAPGQRLEWIGDINPHNGGPIYNQKFTGRATLTVDKSASTAYMELSSLRSEDTAVYYCARGELGHWYFDVWGQGTTVTVSS

SEQ ID NO: 31 - protein, 111 aa, Artificial Sequence
  synthetic construct
DIVLTQSPASLALSPGERATLSCRASKSVSTSGYSYIHWYQQKPGQAPRLLIYLASNLDSGVPARFSGSGSGTDFTLTISSLEEEDFATYYCQHSRDLPPTFGQGTKLEIK

SEQ ID NO: 32 - protein, 117 aa, Artificial Sequence
  synthetic construct
QVQLVQSGAEVKKPGASVKVSCKASGYTFTNYWMHWLRQRPGQGLEWIGNIGPSDSKTHYNQKFKDRVTMTVDKSTSTAYMELSSLRSEDTAVYYCARGDYVLFTYWGQGTLVTVSS

SEQ ID NO: 33 - protein, 107 aa, Artificial Sequence
  synthetic construct
DIVLTQSPATLSLSPGERATLSCRASQSISNNLHWYQQKPGQAPRLLIKYASQSISGIPARFSGSGSGTDFTLTISSLEPEDFAVYYCQQSYSWPLTFGQGTKLEIK

SEQ ID NO: 34 - protein, 5 aa, Artificial Sequence
  synthetic construct
SYGMH

SEQ ID NO: 35 - protein, 17 aa, Artificial Sequence
  synthetic construct
VISYDGSNKYYADSVKG

SEQ ID NO: 36 - protein, 9 aa, Artificial Sequence
  synthetic construct
IGATDPLDY

SEQ ID NO: 37 - protein, 14 aa, Artificial Sequence
  synthetic construct
TGTSSDVGGYNYVS

SEQ ID NO: 38 - protein, 7 aa, Artificial Sequence
  synthetic construct
DVNERSS

SEQ ID NO: 39 - protein, 10 aa, Artificial Sequence
  synthetic construct
ASYAGRYTWM

SEQ ID NO: 40 - protein, 5 aa, Artificial Sequence
  synthetic construct
DYNMD

SEQ ID NO: 41 - protein, 17 aa, Artificial Sequence
  synthetic construct
DINPHNGGPIYNQKFTG

SEQ ID NO: 42 - protein, 10 aa, Artificial Sequence
  synthetic construct
GELGHWYFDV

SEQ ID NO: 43 - protein, 15 aa, Artificial Sequence
  synthetic construct
RASKSVSTSGYSYIH

SEQ ID NO: 44 - protein, 7 aa, Artificial Sequence
  synthetic construct
LASNLDS

SEQ ID NO: 45 - protein, 9 aa, Artificial Sequence
  synthetic construct
QHSRDLPPT

SEQ ID NO: 46 - protein, 5 aa, Artificial Sequence
  synthetic construct
NYWMH

SEQ ID NO: 47 - protein, 17 aa, Artificial Sequence
  synthetic construct
NIGPSDSKTHYNQKFKD

SEQ ID NO: 48 - protein, 8 aa, Artificial Sequence
  synthetic construct
GDYVLFTY

SEQ ID NO: 49 - protein, 11 aa, Artificial Sequence
  synthetic construct
RASQSISNNLH

SEQ ID NO: 50 - protein, 7 aa, Artificial Sequence
  synthetic construct
YASQSIS

SEQ ID NO: 51 - protein, 9 aa, Artificial Sequence
  synthetic construct
QQSYSWPLT

SEQ ID NO: 52 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 107 aa, Homo sapiens [sequence omitted]

SEQ ID NO: 55 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 56 - protein, 449 aa, Artificial Sequence
  synthetic construct
EVQLVQSGAEVKKPGASVKISCKASGYTFTDYNMDWVKQAPGQRLEWIGDINPHNGGPIYNQKFTGRATLTVDKSASTAYMELSSLRSEDTAVYYCARGELGHWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 218 aa, Artificial Sequence
  synthetic construct
DIVLTQSPASLALSPGERATLSCRASKSVSTSGYSYIHWYQQKPGQAPRLLIYLASNLDSGVPARFSGSGSGTDFTLTISSLEEEDFATYYCQHSRDLPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 444 aa, Artificial Sequence
  synthetic construct
QVQLVQSGAEVKKPGASVKVSCKASGYTFTNYWMHWLRQRPGQGLEWIGNIGPSDSKTHYNQKFKDRVTMTVDKSTSTAYMELSSLRSEDTAVYYCARGDYVLFTYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 214 aa, Artificial Sequence
  synthetic construct
DIVLTQSPATLSLSPGERATLSCRASQSISNNLHWYQQKPGQAPRLLIKYASQSISGIPARFSGSGSGTDFTLTISSLEPEDFAVYYCQQSYSWPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0212279 A1
(43) Pub. Date: Jul. 6, 2023

(54) TREATMENT AND PREVENTION OF DISEASE CAUSED BY TYPE IV COLLAGEN DYSFUNCTION

(71) Applicants: Singapore Health Services PTE, LTD., Singapore (SG); National University of Singapore, Singapore (SG)

(72) Inventors: Stuart Alexander Cook, Singapore (SG); Anissa Anindya, Widjaja (SG); Sebastian Schaefer, Singapore (SG)

(73) Assignees: Singapore Health Services PTE, LTD., Singapore (SG); National University of Singapore, Singapore (SG)

(21) Appl. No.: 18/011,147

(22) PCT Filed: Jun. 17, 2021
(86) PCT No.: PCT/EP2021/066446

§ 371 Date: Dec. 16, 2022

(30) Foreign Application Priority Data
Jun. 18, 2020 (GB) 2009292.0

(51) Int. Cl. C07K 16/24; A61P 13/12

(52) U.S. Cl. CPC C07K 16/244; A61P 13/12; C07K 2317/76; A61K 2039/505

(57) ABSTRACT

Methods of treating and preventing Alport syndrome through inhibiting interleukin 11 (EL-11)-mediated signalling are disclosed, as well as agents for use in such methods.

This application claims priority from GB 2009292.0 filed 18 Jun. 2020, the contents and elements of which are herein incorporated by reference for all purposes.

### FIELD OF THE INVENTION

The present invention relates to the diagnosis, treatment and prophylaxis, in particular of diseases caused by type IV collagen dysfunction, such as Alport syndrome.

### BACKGROUND TO THE INVENTION

Alport syndrome (AS) is a progressive hereditary renal disease characterised by glomerulonephritis, ocular abnormalities and sensorineural hearing loss (reviewed e.g. in Nozu et al., Clin Exp Nephrol. (2019) 23(2):158-168).

Alport syndrome is caused by mutations in the COL4A3, COL4A4 and COL4A5 genes encoding type IV collagens^{1,2}, and manifests due to defective production of the glomerular basement membrane, integrin-mediated podocyte dysfunction, glomerular hypertension, and ultrafiltration^{3,4}. Later, affected subjects develop kidney failure. Alport syndrome affects up to 60,000 people in the United States, and is associated with hearing loss, ocular abnormalities, and chronic kidney disease (CKD). Alport syndrome affects 1:5000 children and is characterized by glomerulonephritis, hearing loss, hematuria, proteinuria and end-stage kidney disease. Sometimes, eyes, ears and other parts of the body are affected.

Kidney dysfunction in AS is initiated in the glomerulus, related to altered GBM mechanics and podocyte dysfunction. However, as in other primary glomerular diseases, a major determinant of progressive kidney failure is in the associated tubulointerstitial disease⁴. Indeed, similar to other forms of CKD, kidney function in AS patients correlates most strongly with the degree of tubulointerstitial fibrosis, rather than glomerular pathology¹¹. Disease pathogenesis in AS is complex, involving renin angiotensin system and TGFβ activation, inflammation, partial epithelial-mesenchymal transition (pEMT) and fibrosis, among other factors^{9,12,13}.

In the commonest form of disease due to X-linked mutation of COL4A5, 90% of affected males develop end-stage kidney failure by the age of 40⁵. Early disease can manifest as hematuria, microalbuminuria or proteinuria and while there are no specific therapies, affected children are commonly treated with an angiotensin converting enzyme inhibitor (ACEi), based in part on extrapolation of studies conducted in Col43^{-/-} mice⁶ and supported by data from clinical trials^{7,8}.

The Col4a3^{-/-} mouse strain is widely viewed as one of the best animal models of progressive AS. In seminal studies, treatment of 4-week-old Col4a3^{-/-} mice with an ACEi (ramipril), prior to onset of proteinuria and tubulointerstitial fibrosis, attenuated kidney dysfunction and prolonged lifespan⁶. However, if ramipril treatment of Col4a3^{-/-} mice was delayed until after proteinuria was established, there was no beneficial effect^{6,9}. Even with treatment, AS often progresses to end-stage renal failure. There are no specific or second-line medical therapies for AS and renal transplantation is the preferred treatment for progressive CKD in AS¹⁰. There remains a need for effective treatments for Alport system.

### SUMMARY OF THE INVENTION

In a first aspect, the present invention provides an agent capable of inhibiting interleukin 11 (IL-11)-mediated signalling for use in a method of treating or preventing a disease or condition characterised by type IV collagen dysfunction.

The present invention also provides the use of an agent capable of inhibiting interleukin 11 (IL-11)-mediated signalling in the manufacture of a medicament for use in a method of treating or preventing a disease or condition characterised by type IV collagen dysfunction.

The present invention also provides a method of treating or preventing a disease or condition characterised by type IV collagen dysfunction, comprising administering a therapeutically or prophylactically effective amount of an agent capable of inhibiting interleukin 11 (IL-11)-mediated signalling to a subject.

The present invention also provides an agent capable of inhibiting interleukin 11 (IL-11)-mediated signalling for use in a method of treating or preventing Alport syndrome.

The present invention also provides the use of an agent capable of inhibiting interleukin 11 (IL-11)-mediated signalling in the manufacture of a medicament for use in a method of treating or preventing Alport syndrome.

The present invention also provides a method of treating or preventing Alport syndrome, comprising administering a therapeutically or prophylactically effective amount of an agent capable of inhibiting interleukin 11 (IL-11)-mediated signalling to a subject.

In some embodiments the agent is an agent capable of preventing or reducing the binding of interleukin 11 (IL-11) to a receptor for interleukin 11 (IL-11R).

In some embodiments the agent is capable of binding to interleukin 11 (IL-11) or a receptor for interleukin 11 (IL-11R).

In some embodiments the agent is selected from the group consisting of: an antibody or an antigen-binding fragment thereof, a polypeptide, a peptide, a nucleic acid, an oligonucleotide, an aptamer or a small molecule.

In some embodiments the agent is an antibody or an antigen-binding fragment thereof.

In some embodiments the agent is an anti-IL-11 antibody antagonist of IL-11-mediated signalling, or an antigen-binding fragment thereof.

In some embodiments the antibody or antigen-binding fragment comprises:
(i) a heavy chain variable (VH) region incorporating the following CDRs:
   HC-CDR1 having the amino acid sequence of SEQ ID NO:34
   HC-CDR2 having the amino acid sequence of SEQ ID NO:35
   HC-CDR3 having the amino acid sequence of SEQ ID NO:36; and
(ii) a light chain variable (VL) region incorporating the following CDRs:
   LC-CDR1 having the amino acid sequence of SEQ ID NO:37
   LC-CDR2 having the amino acid sequence of SEQ ID NO:38
   LC-CDR3 having the amino acid sequence of SEQ ID NO:39.

In some embodiments the antibody or antigen-binding fragment comprises:
(i) a heavy chain variable (VH) region incorporating the following CDRs:
   HC-CDR1 having the amino acid sequence of SEQ ID NO:40
   HC-CDR2 having the amino acid sequence of SEQ ID NO:41
   HC-CDR3 having the amino acid sequence of SEQ ID NO:42; and
(ii) a light chain variable (VL) region incorporating the following CDRs:
   LC-CDR1 having the amino acid sequence of SEQ ID NO:43
   LC-CDR2 having the amino acid sequence of SEQ ID NO:44
   LC-CDR3 having the amino acid sequence of SEQ ID NO:45.

In some embodiments the agent is an anti-IL-11Rα antibody antagonist of IL-11-mediated signalling, or an antigen-binding fragment thereof.

In some embodiments the antibody or antigen-binding fragment comprises:
(i) a heavy chain variable (VH) region incorporating the following CDRs:
   HC-CDR1 having the amino acid sequence of SEQ ID NO:46
   HC-CDR2 having the amino acid sequence of SEQ ID NO:47
   HC-CDR3 having the amino acid sequence of SEQ ID NO:48; and
(ii) a light chain variable (VL) region incorporating the following CDRs:
   LC-CDR1 having the amino acid sequence of SEQ ID NO:49
   LC-CDR2 having the amino acid sequence of SEQ ID NO:50
   LC-CDR3 having the amino acid sequence of SEQ ID NO:51.

In some embodiments the agent is a decoy receptor. In some embodiments the agent is a decoy receptor for IL-11. In some embodiments the decoy receptor for IL-11 comprises: (i) an amino acid sequence corresponding to the cytokine binding module of gp130 and (ii) an amino acid sequence corresponding to the cytokine binding module of IL-11Rα.

In some embodiments the agent is an IL-11 mutein. In some embodiments the IL-11 mutein is W147A.

In some embodiments the agent is capable of preventing or reducing the expression of interleukin 11 (IL-11) or a receptor for interleukin 11 (IL-11R). In some embodiments the agent is an oligonucleotide or a small molecule.

In some embodiments the agent is an antisense oligonucleotide capable of preventing or reducing the expression of IL-11. In some embodiments the antisense oligonucleotide capable of preventing or reducing the expression of IL-11 is siRNA targeted to IL11 comprising the sequence of SEQ ID NO:12, 13, 14 or 15.

In some embodiments the agent is an antisense oligonucleotide capable of preventing or reducing the expression of IL-11Rα. In some embodiments the antisense oligonucleotide capable of preventing or reducing the expression of IL-11Rα is siRNA targeted to IL11RA comprising the sequence of SEQ ID NO:16, 17, 18 or 19.

In some embodiments the interleukin 11 receptor is or comprises IL-11Rα.

In some embodiments the method comprises administering the agent to a subject in which expression of interleukin 11 (IL-11) or a receptor for IL-11 (IL-11R) is upregulated.

In some embodiments the method comprises administering the agent to a subject in expression of interleukin 11 (IL-11) or a receptor for interleukin 11 (IL-11R) has been determined to be upregulated.

In some embodiments the method comprises determining whether expression of interleukin 11 (IL-11) or a receptor for IL-11 (IL-11R) is upregulated in the subject and administering the agent to a subject in which expression of interleukin 11 (IL-11) or a receptor for IL-11 (IL-11R) is upregulated.

Description

In the present disclosure the inventors establish IL-11-mediated signalling as a driver of the pathology of Alport syndrome. Expression of IL-11 is found to be upregulated in the kidneys of mice in a model of Alport syndrome, and the receptor for IL11 (IL11RA1) is expressed on podocytes and tubule cells. Treatment of mice having Alport syndrome with antibody antagonist of IL-11-mediated signalling is shown to reduce kidney fibrosis, inflammation and tubule damage, and to improve kidney function and extend lifespan. Thus the present disclosure identifies the IL-11/1L-11 receptor signalling pathway as a therapeutic target for Alport syndrome, and demonstrates that antagonism of IL-11-mediated signalling is a suitable intervention for Alport syndrome.

Interleukin 11 and Receptors for IL-11

Interleukin 11 (IL-11), also known as adipogenesis inhibitory factor, is a pleiotropic cytokine and a member of the IL-6 family of cytokines that includes IL-6, IL-11, IL-27, IL-31, oncostatin M (OSM), leukemia inhibitory factor (LIF), cardiotrophin-1 (CT-1), cardiotrophin-like cytokine (CLC), ciliary neurotrophic factor (CNTF) and neuropoetin (NP-1).

Interleukin 11 (IL-11) is expressed in a variety of mesenchymal cell types. IL-11 genomic sequences have been mapped onto chromosome 19 and the centromeric region of chromosome 7, and is transcribed with a canonical signal peptide that ensures efficient secretion from cells. The activator protein complex of IL-11, cJun/AP-1, located within its promoter sequence is critical for basal transcriptional regulation of IL-11 (Du and Williams., Blood 1997, Vol 89: 3897-3908). The immature form of human IL-11 is a 199 amino acid polypeptide whereas the mature form of IL-11 encodes a protein of 178 amino acid residues (Garbers and Scheller., Biol. Chem. 2013; 394(9):1145-1161). The human IL-11 amino acid sequence is available under UniProt accession no. P20809 (P20809.1 GI:124294; SEQ ID NO:1). Recombinant human IL-11 (oprelvekin) is also commercially available. IL-11 from other species, including mouse, rat, pig, cow, several species of bony fish and primates, have also been cloned and sequenced.

In this specification “IL-11” refers to an IL-11 from any species and includes isoforms, fragments, variants or homologues of an IL-11 from any species. In preferred embodiments the species is human (Homo sapiens). Isoforms, fragments, variants or homologues of an IL-11 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of immature or mature IL-11 from a given species, e.g. human. Isoforms, fragments, variants or homologues of an IL-11 may optionally be characterised by ability to bind IL-11Rα (preferably from the same species) and stimulate signal transduction in cells expressing IL-11Rα and gp130 (e.g. as described in Curtis et al. Blood, 1997, 90(11); or Karpovich et al. Mol. Hum. Reprod. 2003 9(2): 75-80). A fragment of IL-11 may be of any length (by number of amino acids), although may optionally be at least 25% of the length of mature IL-11 and may have a maximum length of one of 50%, 75%, 80%, 85%, 90%, 91%, 92%, 93%, 94%, 95%, 96%, 97%, 98%, or 99% of the length of mature IL-11. A fragment of IL-11 may have a minimum length of 10 amino acids, and a maximum length of one of 15, 20, 25, 30, 40, 50, 60, 70, 80, 90, 100, 110, 120, 130, 140, 150, 160, 170, 180, 190 or 195 amino acids.

IL-11 signals through a homodimer of the ubiquitously expressed glycoprotein 130 (gp130; also known as glycoprotein 130, IL-6ST, IL-6-beta or CD130). Gp130 is a transmembrane protein that forms one subunit of the type I cytokine receptor with the IL-6 receptor family. Specificity is gained through an individual interleukin 11 receptor subunit alpha (IL-11Ra), which does not directly participate in signal transduction, although the initial cytokine binding event to the α-receptor leads to the final complex formation with gp130.

Human gp130 (including the 22 amino acid signal peptide) is a 918 amino acid protein, and the mature form is 866 amino acids, comprising a 597 amino acid extracellular domain, a 22 amino acid transmembrane domain, and a 277 amino acid intracellular domain. The extracellular domain of the protein comprises the cytokine-binding module (CBM) of gp130. The CBM of gp130 comprises the Ig-like domain D1, and the fibronectin-type III domains D2 and D3 of gp130. The amino acid sequence of human gp130 is available under UniProt accession no. P40189-1 (SEQ ID NO:2).

Human IL-11Rα is a 422 amino acid polypeptide (UniProt Q14626; SEQ ID NO:3) and shares ˜85% nucleotide and amino acid sequence identity with the murine IL-11Rα. Two isoforms of IL-11Rα have been reported, which differ in the cytoplasmic domain (Du and Williams, supra). The IL-11 receptor α-chain (IL-11Rα) shares many structural and functional similarities with the IL-6 receptor α-chain (IL-6Rα). The extracellular domain shows 24% amino acid identity including the characteristic conserved Trp-Ser-X-Trp-Ser (WSXWS) motif. The short cytoplasmic domain (34 amino acids) lacks the Box 1 and 2 regions that are required for activation of the JAK/STAT signalling pathway.

The receptor binding sites on murine IL-11 have been mapped and three sites—sites I, II and III—identified. Binding to gp130 is reduced by substitutions in the site II region and by substitutions in the site III region. Site III mutants show no detectable agonist activity and have IL-11Rα antagonist activity (Cytokine Inhibitors Chapter 8; edited by Gennaro Ciliberto and Rocco Savino, Marcel Dekker, Inc. 2001).

In this specification a receptor for IL-11 (IL-11R) refers to a polypeptide or polypeptide complex capable of binding IL-11. In some embodiments an IL-11 receptor is capable of binding IL-11 and inducing signal transduction in cells expressing the receptor.

An IL-11 receptor may be from any species and includes isoforms, fragments, variants or homologues of an IL-11 receptor from any species. In preferred embodiments the species is human (Homo sapiens).

In some embodiments the IL-11 receptor may be IL-11Rα. In some embodiments a receptor for IL-11 may be a polypeptide complex comprising IL-11Rα. In some embodiments the IL-11 receptor may be a polypeptide complex comprising IL-11Rα and gp130. In some embodiments the IL-11 receptor may be gp130 or a complex comprising gp130 to which IL-11 binds.

Isoforms, fragments, variants or homologues of an IL-11Rα may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of IL-11Rα from a given species, e.g. human. Isoforms, fragments, variants or homologues of an IL-11Rα may optionally be characterised by ability to bind IL-11 (preferably from the same species) and stimulate signal transduction in cells expressing the IL-11Rα and gp130 (e.g. as described in Curtis et al. Blood, 1997, 90(11) or Karpovich et al. Mol. Hum. Reprod. 2003 9(2): 75-80). A fragment of an IL-11 receptor may be of any length (by number of amino acids), although may optionally be at least 25% of the length of the mature IL-11Rα and have a maximum length of one of 50%, 75%, 80%, 85%, 90%, 91%, 92%, 93%, 94%, 95%, 96%, 97%, 98%, or 99% of the length of the mature IL-11Rα. A fragment of an IL-11 receptor fragment may have a minimum length of 10 amino acids, and a maximum length of one of 15, 20, 25, 30, 40, 50, 100, 110, 120, 130, 140, 150, 160, 170, 180, 190, 200, 250, 300, 400, or 415 amino acids.

### IL-11 Signalling

IL-11 binds to IL-11Rα with low affinity (Kd ˜22 nM; see Metcalfe et al., JBC (2020) Manuscript RA119.012351), and interaction between these binding partners alone is insufficient to transduce a biological signal. The generation of a high affinity receptor (Kd ˜400 to 800 pmol/L) capable of signal transduction requires co-expression of the IL-11Rα and gp130 (Curtis et al Blood 1997; 90 (11):4403-12; Hilton et al., EMBO J 13:4765, 1994; Nandurkar et al., Oncogene 12:585, 1996). Binding of IL-11 to cell-surface IL-11Rα induces heterodimerization, tyrosine phosphorylation, activation of gp130 and downstream signalling, predominantly through the mitogen-activated protein kinase (MAPK)-cascade and the Janus kinase/signal transducer and activator of transcription (Jak/STAT) pathway (Garbers and Scheller, supra).

In principle, a soluble IL-11Rα can also form biologically active soluble complexes with IL-11 (Pflanz et al., 1999 FEBS Lett, 450, 117-122) raising the possibility that, similar to IL-6, IL-11 may in some instances bind soluble IL-11Rα prior to binding cell-surface gp130 (Garbers and Scheller, supra). Curtis et al (Blood 1997 Dec. 1; 90 (11):4403-12) describe expression of a soluble murine IL-11 receptor alphα chain (sIL-11R) and examined signalling in cells expressing gp130. In the presence of gp130 but not transmembrane IL-11R the sIL-11R mediated IL-11 dependent differentiation of M1 leukemic cells and proliferation in Ba/F3 cells and early intracellular events including phosphorylation of gp130, STAT3 and SHP2 similar to signalling through transmembrane IL-11R. Activation of signalling through cell-membrane bound gp130 by IL-11 bound to soluble IL-11Rα has recently been demonstrated (Lokau et al., 2016 Cell Reports 14, 1761-1773). This so-called IL-11 trans signalling may be important for disease pathogenesis, yet its role in human disease has not yet been studied.

As used herein, ‘IL-11 trans signalling’ is used to refer to signalling which is triggered by binding of IL-11 bound to IL-11Rα, to gp130. The IL-11 may be bound to IL-11Rα as a non-covalent complex. The gp130 is membrane-bound and expressed by the cell in which signalling occurs following binding of the IL-11:IL-11Rα complex to gp130. In some embodiments the IL-11Rα may be a soluble IL-11Rα. In some embodiments, the soluble IL-11Rα is a soluble (secreted) isoform of IL-11Rα (e.g. lacking a transmembrane domain). In some embodiments, the soluble IL-11Rα is the liberated product of proteolytic cleavage of the extracellular domain of cell membrane bound IL-11Rα. In some embodiments, the IL-11Rα may be cell membrane-bound, and signalling through gp130 may be triggered by binding of IL-11 bound to cell-membrane-bound IL-11Rα, termed “IL-11 cis signalling”. In preferred embodiments, inhibition of IL-11-mediated signalling is achieved by disrupting IL-11-mediated cis signalling.

IL-11-mediated signalling has been shown to stimulate hematopoiesis and thrombopoiesis, stimulate osteoclast activity, stimulate neurogenesis, inhibit adipogenesis, reduce pro inflammatory cytokine expression, modulate extracellular matrix (ECM) metabolism, and mediate normal growth control of gastrointestinal epithelial cells (Du and Williams, supra).

The physiological role of Interleukin 11 (IL-11) remains unclear. IL-11 has been most strongly linked with activation of haematopoetic cells and with platelet production. IL-11 has also been shown to confer protection against graft-vs-host-disease, inflammatory arthritis and inflammatory bowel disease, leading to IL-11 being considered an anti-inflammatory cytokine (Putoczki and Ernst, J Leukoc Biol 2010, 88(6):1109-1117). However, it is suggested that IL-11 is pro-inflammatory as well as anti-inflammatory, pro-angiogenic and important for neoplasia. Recent studies have shown that IL-11 is readily detectable during viral-induced inflammation in a mouse arthritis model and in cancers, suggesting that the expression of IL-11 can be induced by pathological stimuli. IL-11 is also linked to Stat3-dependent activation of tumour-promoting target genes in neoplastic gastrointestinal epithelium (Putoczki and Ernst, supra).

As used herein, “IL-11 signalling” and “IL-11-mediated signalling” refers to signalling mediated by binding of IL-11, or a fragment thereof having the function of the mature IL-11 molecule, to a receptor for IL-11. It will be appreciated that “IL-11 signalling” and “IL-11 mediated signalling” refer to signalling initiated by IL-11/functional fragment thereof, e.g. through binding to a receptor for IL-11. “Signalling” in turn refers to signal transduction and other cellular processes governing cellular activity.

Type IV Collagen Dysfunction and Alport Syndrome

The present invention is concerned with the treatment and/or prevention of diseases and conditions characterised by type IV collagen dysfunction.

Diseases and conditions characterised by type IV collagen dysfunction are described e.g. in Pescucci et al., J Nephrol (2003) 16(2): 314-316, which is hereby incorporated by reference in its entirety.

As used herein, ‘dysfunction’ encompasses all abnormal—i.e. non-wildtype—function, including aberrant function and insufficient function. Herein, ‘wildtype function’ refers to the quality and level of type IV collagen function observed in a subject not having a disease/condition characterised by type IV collagen dysfunction. For example, wildtype type IV collagen function may be the quality and level of type IV collagen function of a subject having the wildtype alleles for genes encoding type IV collagen α chains.

Diseases and conditions characterised by type IV collagen dysfunction may be characterised by disrupted/abnormal formation and/or function of type IV collagen complexes. For example, diseases and conditions characterised by type IV collagen dysfunction may be characterised by type IV collagen insufficiency, i.e. insufficiency of a structure/function performed by type IV collagen complexes.

Such diseases and conditions may be characterised by one or more of the following relative to the wildtype state: a reduction in the number of type IV collagen α chains, a reduction in the number of trimeric type IV collagen complexes, a reduction of the proportion of type IV collagen α chains associating to form trimeric type IV collagen complexes, a reduction in the level of a correlate of trimeric type IV collagen complex function, a thinner or incomplete basement membrane (e.g. glomerular basement membrane), or a thinner or incomplete lamina densa.

In particular, the present invention is concerned with the treatment and/or prevention of Alport syndrome. That is, in some embodiments, the disease/condition characterised by type IV collagen dysfunction is Alport syndrome.

Alport syndrome is reviewed e.g. in Nozu et al., Clin Exp Nephrol. (2019) 23(2):158-168, which is hereby incorporated by reference in its entirety. Alport syndrome (AS) is a progressive hereditary renal disease characterised by glomerulonephritis, ocular abnormalities and sensorineural hearing loss.

Alport syndrome is caused by pathogenic variants of genes encoding type IV collagen α chains, specifically α3, α4, and α5 chains. Type IV collagen has 6 different α chains (α1 to α6) which form triple helix structures in which the three chains are combined. The combination of three α-chains is organ-specific, and in the glomerular basement membrane (GBM), cochlea basement membrane, and at the base of the ocular lens, the triplet is α3, α4, α5. In Bowman's capsule and skin basement membrane, the triplet is α5, α5, α6. Alport syndrome-associated variants disrupt formation of triple helix structures, resulting in the thinning and splitting of basal lamina formed by type IV collagen, giving rise to nephropathy, sensorineural hearing loss, and eye lesions.

Alport syndrome is divided into three classes based on mode of inheritance: X-linked Alport syndrome (XLAS), autosomal recessive AS (ARAS), and autosomal dominant AS (ADAS). XLAS is caused by genetic variants of COL4A5 encoding type IV collagen a5. ADAS and ARAS are caused by genetic variants of the COL4A3 and/or COL4A4 genes respectively encoding type IV collagens a3 and a4. About 80% of Alport syndrome is XLAS, about 15% is ARAS, and about 5% is ADAS.

A subject having a Alport syndrome may have a symptom/correlate of Alport syndrome. A subject having Alport syndrome may have been diagnosed as having Alport syndrome. A subject may satisfy the diagnostic criteria for the diagnosis of Alport syndrome.

Alport syndrome may be diagnosed in accordance with the February 2015 diagnostic features of Alport syndrome prepared by the Working Group on Alport Syndrome of the Japanese Society of Pediatric Nephrology (Nakanishi and Yoshikawa, Nihon Jinzo Gakkai Shi. (2015) 57(4):736-42):
I. Primary feature:
I-1. Persistent hematuria (i.e. lasting for more than 3 months, and confirmed by urinalysis on at least two occasions)
II. Secondary features:
II-1. Mutation in a type IV collagen gene
   E.g. a homozygous or heterozygous mutation of COL4A3 or COL4A4, or a hemizygous (male) or heterozygous (female) mutation of COL4A5.
II-2. Abnormal expression of type IV collagen
   E.g. as determined by immunostaining using anti-α chain antibodies. For example, glomeruli, Bowman's capsule, and the skin basement membranes of male patients with XLAS may stain completely negative using an anti-α5-chain antibody.
II-3. Glomerular basement membrane (GBM)-specific abnormalities
   E.g. broad irregular thickening of the GBM, reticulation of the lamina densa, or extensive thinning of the GBM.
III. Accessory features:
III-1. Family history of kidney disease
III-2. Bilateral sensorineural deafness
III-3. Ocular abnormalities
   E.g. anterior lenticonus, posterior subcapsular cataract, posterior polymorphous dystrophy, or retinal flecks.
III-4. Diffuse leiomyomatosis

In some embodiments a subject having a disease/condition characterised by type IV collagen dysfunction (e.g. Alport syndrome) may have one or more of: nephritis, glomerulonephritis, hematuria, proteinuria, a mutation in a type IV collagen gene (e.g. a homozygous or heterozygous mutation of COL4A3 or COL4A4, or a hemizygous or heterozygous mutation of COL4A5), abnormal expression of type IV collagen (e.g. as determined by immunostaining using an anti-α chain antibody), podocyte dysfunction, a GBM abnormality (e.g. broad irregular thickening of the GBM, reticulation of the lamina densa, or extensive thinning of the GBM), family history of kidney disease, bilateral sensorineural deafness, an ocular abnormality (e.g. anterior lenticonus, posterior subcapsular cataract, posterior polymorphous dystrophy, or retinal flecks), and diffuse leiomyomatosis.

In some embodiments, a subject having a disease/condition characterised by type IV collagen dysfunction (e.g. Alport syndrome) may have a genetic variant which is associated with the disease/condition characterised by type IV collagen dysfunction (e.g. Alport syndrome).

As used herein, a ‘genetic variant’ refers to a variant of the nucleotide sequence of a reference nucleic acid sequence relative to the most common nucleotide sequence. For example, a genetic variant may be an allele of a gene comprising a nucleotide sequence which is non-identical to the nucleotide sequence of the most common allele of the gene. The most common allele of the gene may be referred to as the wildtype allele. Examples of genetic variants include e.g. mutations (substitutions, insertions, deletions) and single nucleotide polymorphisms (SNPs).

Genetic variants which are ‘associated with’ a given disease/condition may be genetic variants which are causal for, or which exacerbate the symptoms of, the disease/condition, or may be genetic variants which are risk factors for the development or progression of the disease/condition. Genetic variants which are associated with a given disease/condition may also be referred to herein as ‘disease-associated variants’. A disease-associated variant may also be referred to herein as ‘disease-associated allele’.

In some embodiments, a subject having a disease/condition characterised by type IV collagen dysfunction (e.g. Alport syndrome) may have a genetic variant of a gene encoding a type IV collagen a chain, e.g. a variant of one of COL4A1, COL4A2, COL4A3, COL4A4, COL4A5 and COL4A6. In some embodiments, a subject having a disease/condition characterised by type IV collagen dysfunction (e.g. Alport syndrome) may have a genetic variant of one of COL4A3, COL4A4 and COL4A5.

In some embodiments, the genetic variant may be a variant which is known, or which is predicted, to result in disrupted/abnormal formation of trimeric type IV collagen complexes. For example, the variant may encode a non-functional and/or dysfunctional version of the type IV collagen α chain, or may encode a version of the type IV collagen α chain which is truncated, misfolded and/or degraded.

In some embodiments the genetic variant of a gene encoding a type IV collagen α chain is associated with one or more of: reduced gene or protein expression of the type IV collagen α chain, a reduced level of RNA encoding the type IV collagen α chain, reduced transcription of nucleic acid encoding the type IV collagen α chain, increased degradation of RNA encoding the type IV collagen α chain, reduced or altered post-transcriptional processing (e.g. splicing, translation, post-translational processing) of RNA encoding the type IV collagen α chain, a reduced protein level of the type IV collagen α chain, increased degradation of the type IV collagen α chain, reduced or altered post-translational processing of the type IV collagen α chain, and reduced or altered association of the type IV collagen α chain with other type IV collagen α chains.

In some embodiments, a subject having a disease/condition characterised by type IV collagen dysfunction (e.g. Alport syndrome) may have an Alport syndrome-associated genetic variant.

In some embodiments, an Alport syndrome-associated genetic variant may be a variant of a gene encoding a type IV collagen α chain, e.g. a variant of one of COL4A1, COL4A2, COL4A3, COL4A4, COL4A5 and COL4A6. In some embodiments, an Alport syndrome-associated genetic variant is a variant of one of COL4A3, COL4A4 and COL4A5.

In some embodiments, an Alport syndrome-associated genetic variant is a variant described e.g. in Savige et al., PLoS One (2016) 11(9): e0161802, which is hereby incorporated by reference in its entirety. In some embodiments, an Alport syndrome-associated genetic variant is selected from: one of the following variants of COL4A5: tandem duplication of 35 exons described in Arrondel et al., Kidney Int (2004) 65: 2030-2040 (hereby incorporated by reference in its entirety), p.Gly325Arg, p.Arg373*, p.Gly624Asp, p.Gly869Arg, p.Ser916Gly, p.Gly953Val, p.Gly1030Ser, p.Arg1569Gln, p.Leu1655Arg, p.Arg1683Gln, or p. Arg1683*; one of the following variants of COL4A3: c.40-63del, p.Gly43Arg, p.Glu162Gly, p.Gly695Arg, p.Gly871Cys, p.Gly1334Glu, p. Gln1495Arg, or p. 13_22 del LPLLLVLL; or one of the following variants of COL4A4: p.Gly545Ala, c.2384-5T>C, p.A880Hisfs69*, p.Gly960Arg, or p.Ser969* (wherein “*” denotes the introduction of a stop codon).

Agents Capable of Inhibiting the Action of IL-11

Aspects of the present invention involve inhibition of IL-11-mediated signalling.

Herein, ‘inhibition’ refers to a reduction, decrease or lessening relative to a control condition. For example, inhibition of the action of IL-11 by an agent capable of inhibiting IL-11-mediated signalling refers to a reduction, decrease or lessening of the extent/degree of IL-11-mediated signalling in the absence of the agent, and/or in the presence of an appropriate control agent.

Inhibition may herein also be referred to as neutralisation or antagonism. That is, an agent capable of inhibiting IL-11-mediated signalling (e.g. interaction, signalling or other activity mediated by IL-11 or an IL-11-containing complex) may be said to be a ‘neutralising’ or ‘antagonist’ agent with respect to the relevant function or process. For example, an agent which is capable of inhibiting IL-11-mediated signalling may be referred to as an agent which is capable of neutralising IL-11-mediated signalling, or may be referred to as an antagonist of IL-11-mediated signalling.

The IL-11 signalling pathway offers multiple routes for inhibition of IL-11 signalling. An agent capable of inhibiting IL-11-mediated signalling may do so e.g. through inhibiting the action of one or more factors involved in, or necessary for, signalling through a receptor for IL-11.

For example, inhibition of IL-11 signalling may be achieved by disrupting interaction between IL-11 (or an IL-11 containing complex, e.g. a complex of IL-11 and IL-11Ra) and a receptor for IL-11 (e.g. IL-11Rα, a receptor complex comprising IL-11Rα, gp130 or a receptor complex comprising IL-11Rα and gp130). In some embodiments, inhibition of IL-11-mediated signalling is achieved by inhibiting the gene or protein expression of one or more of e.g. IL-11, IL-11Rα and gp130.

Inhibition of IL-11-mediated signalling may also be achieved by disrupting interaction between IL-11:11 receptor complexes (i.e. complexes comprising IL-11 and IL-11Rα, or IL-11 and gp130, or IL-11, IL-11Rα and gp130) to form multimers (e.g. hexameric complexes) required for activation of downstream signalling by cells expressing IL-11 receptors.

In embodiments, inhibition of IL-11-mediated signalling is achieved by disrupting IL-11-mediated cis signalling but not disrupting IL-11-mediated trans signalling, e.g. inhibition of IL-11-mediated signalling is achieved by inhibiting gp130-mediated cis complexes involving membrane bound IL-11Rα. In embodiments, inhibition of IL-11-mediated signalling is achieved by disrupting IL-11-mediated trans signalling but not disrupting IL-11-mediated cis signalling, i.e. inhibition of IL-11-mediated signalling is achieved by inhibiting gp130-mediated trans signalling complexes such as IL-11 bound to soluble IL-11Rα or IL-6 bound to soluble IL-6R. In embodiments, inhibition of IL-11-mediated signalling is achieved by disrupting IL-11-mediated cis signalling and IL-11-mediated trans signalling. Any agent as described herein may be used to inhibit IL-11-mediated cis and/or trans signalling.

In other examples, inhibition of IL-11 signalling may be achieved by disrupting signalling pathways downstream of IL-11/1L-11Ra/gp130. That is, in some embodiments inhibition/antagonism of IL-11-mediated signalling comprises inhibition of a signalling pathway/process/factor downstream of signalling through the IL-11/IL-11 receptor complex.

In some embodiments inhibition/antagonism of IL-11-mediated signalling comprises inhibition of signalling through an intracellular signalling pathway which is activated by the IL-11/1L-11 receptor complex. In some embodiments inhibition/antagonism of IL-11-mediated signalling comprises inhibition of one or more factors whose expression/activity is upregulated as a consequence of signalling through the IL-11/IL-11 receptor complex.

In some embodiments, the methods of the present invention employ agents capable of inhibiting JAK/STAT signalling. In some embodiments, agents capable of inhibiting JAK/STAT signalling are capable of inhibiting the action of JAK1, JAK2, JAK3, TYK2, STAT1, STAT2, STAT3, STAT4, STAT5A, STAT5B and/or STAT6. For example, agents may be capable of inhibiting activation of JAK/STAT proteins, inhibiting interaction of JAK or STAT proteins with cell surface receptors e.g. IL-11Rα or gp130, inhibiting phosphorylation of JAK proteins, inhibiting interaction between JAK and STAT proteins, inhibiting phosphorylation of STAT proteins, inhibiting dimerization of STAT proteins, inhibiting translocation of STAT proteins to the cell nucleus, inhibiting binding of STAT proteins to DNA, and/or promoting degradation of JAK and/or STAT proteins. In some embodiments, a JAK/STAT inhibitor is selected from Ruxolitinib (Jakafi/Jakavi; Incyte), Tofacitinib (Xeljanz/Jakvinus; NIH/Pfizer), Oclacitinib (Apoquel), Baricitinib (Olumiant; Incyte/Eli Lilly), Filaotinib (G-146034/GLPG-0634; Galapagos NV), Gandotinib (LY-2784544; Eli Lilly), Lestaurtinib (CEP-701; Teva), Momelotinib (GS-0387/CYT-387; Gilead Sciences), Pacritinib (SB1518; CTI), PF-04965842 (Pfizer), Upadacitinib (ABT-494; AbbVie), Peficitinib (ASP015K/JNJ-54781532; Astellas), Fedratinib (SAR302503; Celgene), Cucurbitacin I (JSI-124) and CHZ868.

In some embodiments, the methods of the present invention employ agents capable of inhibiting MAPK/ERK signalling. In some embodiments, agents capable of inhibiting MAPK/ERK signalling are capable of inhibiting the action of GRB2, inhibiting the action of RAF kinase, inhibiting the action of MEK proteins, inhibiting the activation of MAP3K/MAP2K/MAPK and/or Myc, and/or inhibiting the phosphorylation of STAT proteins. In some embodiments, agents capable of inhibiting ERK signalling are capable of inhibiting ERK p42/44. In some embodiments, an ERK inhibitor is selected from SCH772984, SC1, VX-11e, DEL-22379, Sorafenib (Nexavar; Bayer/Onyx), SB590885, PLX4720, XL281, RAF265 (Novartis), encorafenib (LGX818/Braftovi; Array BioPharma), dabrafenib (Tafinlar; GSK), vemurafenib (Zelboraf; Roche), cobimetinib (Cotellic; Roche), C1-1040, PD0325901, Binimetinib (MEK162/MEKTOVI; Array BioPharma), selumetinib (AZD6244; Array/AstraZeneca) and Trametinib (GSK1120212/Mekinist; Novartis). In some embodiments, the methods of the present invention employ agents capable of inhibiting c-Jun N-terminal kinase (JNK) signalling/activity. In some embodiments, agents capable of inhibiting JNK signalling/activity are capable of inhibiting the action and/or phosphorylation of a JNK (e.g. JNK1, JNK2). In some embodiments, a JNK inhibitor is selected from SP600125, CEP 1347, TCS JNK 6o, c-JUN peptide, SU3327, AEG 3482, TCS JNK 5a, B178D3, IQ3, SR3576, IQ1S, JIP-1 (153-163) and CC401 dihydrochloride.

In the present Examples the inventors demonstrate that NOX4 expression and activity is upregulated by signalling through IL-11/IL-11Rα/gp130. NOX4 is an NADPH oxidase, and a source of reactive oxygen species (ROS). Expression of Nox4 is upregulated in transgenic mice with hepatocyte-specific II11 expression, and primary human hepatocytes stimulated with IL11 upregulate NOX4 expression.

In some embodiments, the present invention employs agents capable of inhibiting NOX4 expression (gene or protein expression) or function. In some embodiments, the present invention employs agents capable of inhibiting IL-11-mediated upregulation of NOX4 expression/function. Agents capable of inhibiting NOX4 expression or function may be referred to herein as NOX4 inhibitors. For example, a NOX4 inhibitor may be capable of reducing expression (e.g. gene and/or protein expression) of NOX4, reducing the level of RNA encoding NOX4, reduce the level of NOX4 protein, and/or reducing the level of a NOX4 activity (e.g. reducing NOX4-mediated NADPH oxidase activity and/or NOX4-mediated ROS production).

NOX4 inhibitors include a NOX4-binding molecules and molecules capable of reducing NOX4 expression. NOX4-binding inhibitors include peptide/nucleic acid aptamers, antibodies (and antibody fragments) and fragments of interaction partners for NOX4 which behave as antagonists of NOX4 function, and small molecules inhibitors of NOX4. Molecules capable of reducing NOX4 expression include antisense RNA (e.g. siRNA, shRNA) to NOX4. In some embodiments, a NOX4 inhibitor is selected from a NOX4 inhibitor described in Altenhofer et al., Antioxid Redox Signal. (2015) 23(5): 406-427 or Augsburder et al., Redox Biol. (2019) 26: 101272, such as GKT137831.

Binding Agents

In some embodiments, agents capable of inhibiting IL-11-mediated signalling may bind to IL-11. In some embodiments, agents capable of inhibiting IL-11-mediated signalling may bind to a receptor for IL-11 (e.g. IL-11Rα, gp130, or a complex containing IL-11Rα and/or gp130). Binding of such agents may inhibit IL-11-mediated signalling by reducing/preventing the ability of IL-11 to bind to receptors for IL-11, thereby inhibiting downstream signalling. Binding of such agents may inhibit IL-11 mediated cis and/or trans-signalling by reducing/preventing the ability of IL-11 to bind to receptors for IL-11, e.g. IL-11Rα and/or gp130, thereby inhibiting downstream signalling. Agents may bind to trans-signalling complexes such as IL-11 and soluble IL-11Rα and inhibit gp130-mediated signalling.

Agents capable of binding to IL-11/an IL-11 containing complex or a receptor for IL-11 may be of any kind, but in some embodiments the agent may be an antibody, an antigen-binding fragment thereof, a polypeptide, a peptide, a nucleic acid, an oligonucleotide, an aptamer or a small molecule. The agents may be provided in isolated or purified form, or may be formulated as a pharmaceutical composition or medicament.

Antibodies and Antigen-Binding Fragments

In some embodiments, an agent capable of binding to IL-11/an IL-11 containing complex or a receptor for IL-11 is an antibody, or an antigen-binding fragment thereof. In some embodiments, an agent capable of binding to IL-11/an IL-11 containing complex or a receptor for IL-11 is a polypeptide, e.g. a decoy receptor molecule. In some embodiments, an agent capable of binding to IL-11/an IL-11 containing complex or a receptor for IL-11 may be an aptamer.

In some embodiments, an agent capable of binding to IL-11/an IL-11 containing complex or a receptor for IL-11 is an antibody, or an antigen-binding fragment thereof. An “antibody” is used herein in the broadest sense, and encompasses monoclonal antibodies, polyclonal antibodies, monospecific and multispecific antibodies (e.g., bispecific antibodies), and antibody fragments, as long as they display binding to the relevant target molecule.

In view of today's techniques in relation to monoclonal antibody technology, antibodies can be prepared to most antigens. The antigen-binding portion may be a part of an antibody (for example a Fab fragment) or a synthetic antibody fragment (for example a single chain Fv fragment [ScFv]). Monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimaeric antibodies are discussed by Neuberger et al (1988, 8th International Biotechnology Symposium Part 2, 792-799). Monoclonal antibodies (mAbs) are particularly useful in the methods of the invention, and are a homogenous population of antibodies specifically targeting a single epitope on an antigen.

Polyclonal antibodies are also useful in the methods of the invention. Monospecific polyclonal antibodies are preferred. Suitable polyclonal antibodies can be prepared using methods well known in the art.

Antigen-binding fragments of antibodies, such as Fab and Fab2 fragments may also be used/provided as can genetically engineered antibodies and antibody fragments. The variable heavy (VH) and variable light (VL) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855).

Antibodies and antigen-binding fragments according to the present disclosure comprise the complementarity-determining regions (CDRs) of an antibody which is capable of binding to the relevant target molecule (i.e. IL-11/an IL-11 containing complex/a receptor for IL-11).

Antibodies capable of binding to IL-11 include e.g. monoclonal mouse anti-human IL-11 antibody clone #22626; Catalog No. MAB218 (R&D Systems, MN, USA), used e.g. in Bockhorn et al. Nat. Commun. (2013) 4(0):1393, clone 6D9A (Abbiotec), clone KT8 (Abbiotec), clone M3103F11 (BioLegend), clone 1F1 (Abnova Corporation), clone 3C6 (Abnova Corporation), clone GF1 (LifeSpan Biosciences), clone 13455 (Source BioScience), 11h3/19.6.1 (Hermann et al., Arthritis Rheum. (1998) 41(8):1388-97), AB-218-NA (R&D Systems), X203 (Ng et al., Sci Transl Med. (2019) 11(511) pii: eaaw1237) and anti-IL-11 antibodies disclosed in US 2009/0202533 A1, WO 99/59608 A2, WO 2018/109174 A2 and WO 2019/238882 A1.

In particular, anti-IL-11 antibody clone 22626 (also known as MAB218) has been shown to be an antagonist of IL-11 mediated signalling, e.g. in Schaefer et al., Nature (2017) 552(7683):110-115. Monoclonal antibody 11h3/19.6.1 is disclosed in Hermann et al., Arthritis Rheum. (1998) 41(8):1388-97 to be a neutralising anti-IL-11 IgG1. AB-218-NA from R&D Systems, used e.g. in McCoy et al., BMC Cancer (2013) 13:16, is another example of neutralizing anti-IL-11 antibody. WO 2018/109174 A2 and WO 2019/238882 A1 disclose yet further exemplary anti-IL-11 antibody antagonists of IL-11 mediated signalling. X203 (also referred to as Enx203) disclosed in Ng, et al., “IL-11 is a therapeutic target in idiopathic pulmonary fibrosis.” bioRxiv 336537; doi: https://doi.org/10.1101/336537 and WO 2019/238882 A1 is an anti-IL-11 antibody antagonist of IL-11-mediated signalling, and comprises the VH region according to SEQ ID NO:92 of WO 2019/238882 A1 (SEQ ID NO:22 of the present disclosure), and the VL region according to SEQ ID NO:94 of WO 2019/238882 A1 (SEQ ID NO:23 of the present disclosure). Humanised versions of the X203 are described in WO 2019/238882 A1, including hEnx203 which comprises the VH region according to SEQ ID NO:117 of WO 2019/238882 A1 (SEQ ID NO:30 of the present disclosure), and the VL region according to SEQ ID NO:122 of WO 2019/238882 Al (SEQ ID NO:31 of the present disclosure). Enx108A is a further example of an anti-IL-11 antibody antagonist of IL-11-mediated signalling, and comprises the VH region according to SEQ ID NO:8 of WO 2019/238882 A1 (SEQ ID NO:26 of the present disclosure), and the VL region according to SEQ ID NO:20 of WO 2019/238882 A1 (SEQ ID NO:27 of the present disclosure).

Antibodies capable of binding to IL-11Rα include e.g. monoclonal antibody clone 025 (Sino Biological), clone EPR5446 (Abcam), clone 473143 (R & D Systems), clones 8E2, 8D10 and 8E4 and the affinity-matured variants of 8E2 described in US 2014/0219919 A1, the monoclonal antibodies described in Blanc et al (J. Immunol Methods. 2000 Jul. 31; 241(1-2);43-59), X209 (Widjaja et al., Gastroenterology (2019) 157(3):777-792, which is also published as Widjaja, et al., “IL-11 neutralising therapies target hepatic stellate cell-induced liver inflammation and fibrosis in NASH.” bioRxiv 470062; doi: https://doi.org/10.1101/470062) antibodies disclosed in WO 2014121325 A1 and US 2013/0302277 A1, and anti-IL-11Rα antibodies disclosed in US 2009/0202533 A1, WO 99/59608 A2, WO 2018/109170 A2 and WO 2019/238884 A1.

In particular, anti-IL-11Rα antibody clone 473143 (also known as MAB1977) has been shown to be an antagonist of IL-11 mediated signalling, e.g. in Schaefer et al., Nature (2017) 552(7683):110-115. US 2014/0219919 A1 provides sequences for anti-human IL-11Rα antibody clones 8E2, 8D10 and 8E4, and discloses their ability to antagonise IL-11 mediated signalling — see e.g. [0489] to [0490] of US 2014/0219919 A1. US 2014/0219919 A1 moreover provides sequence information for an additional 62 affinity-matured variants of clone 8E2, 61 of which are disclosed to antagonise IL-11 mediated signalling—see Table 3 of US 2014/0219919 A1. WO 2018/109170 A2 and WO 2019/238884 A1 disclose yet further exemplary anti-IL-11Rα antibody antagonists of IL-11 mediated signalling. X209 (also referred to as Enx209) disclosed in Widjaja, et al., “IL-11 neutralising therapies target hepatic stellate cell-induced liver inflammation and fibrosis in NASH.” bioRxiv 470062; doi: https://doi.org/10.1101/470062 and WO 2019/238884 A1 is an anti-IL-11Rα antibody antagonist of IL-11-mediated signalling, and comprises the VH region according to SEQ ID NO:7 of WO 2019/238884 A1 (SEQ ID NO:24 of the present disclosure), and the VL region according to SEQ ID NO:14 of WO 2019/238884 A1 (SEQ ID NO:25 of the present disclosure). Humanised versions of the X209 are described in WO 2019/238884 A1, including hEnx209 which comprises the VH region according to SEQ ID NO:11 of WO 2019/238884 A1 (SEQ ID NO:32 of the present disclosure), and the VL region according to SEQ ID NO:17 of WO 2019/238884 A1 (SEQ ID NO:33 of the present disclosure).

The skilled person is well aware of techniques for producing antibodies suitable for therapeutic use in a given species/subject. For example, procedures for producing antibodies suitable for therapeutic use in humans are described in Park and Smolen Advances in Protein Chemistry (2001) 56: 369-421 (hereby incorporated by reference in its entirety).

Antibodies to a given target protein (e.g. IL-11 or IL-11Rα) can be raised in model species (e.g. rodents, lagomorphs), and subsequently engineered in order to improve their suitability for therapeutic use in a given species/subject. For example, one or more amino acids of monoclonal antibodies raised by immunisation of model species can be substituted to arrive at an antibody sequence which is more similar to human germline immunoglobulin sequences (thereby reducing the potential for anti-xenogenic antibody immune responses in the human subject treated with the antibody). Modifications in the antibody variable domains may focus on the framework regions in order to preserve the antibody paratope. Antibody humanisation is a matter of routine practice in the art of antibody technology, and is reviewed e.g. in Almagro and Fransson, Frontiers in Bioscience (2008) 13:1619-1633, Safdari et al., Biotechnology and Genetic Engineering Reviews (2013) 29(2): 175-186 and Lo et al., Microbiology Spectrum (2014) 2(1), all of which are hereby incorporated by reference in their entirety. The requirement for humanisation can be circumvented by raising antibodies to a given target protein (e.g. IL-11 or IL-11Ra) in transgenic model species expressing human immunoglobulin genes, such that the antibodies raised in such animals are fully-human (described e.g. in Bruggemann et al., Arch Immunol Ther Exp (Warsz) (2015) 63(2):101-108, which is hereby incorporated by reference in its entirety).

Phage display techniques may also be employed to the identification of antibodies to a given target protein (e.g. IL-11 or IL-11Rα), and are well known to the skilled person. The use of phage display for the identification of fully human antibodies to human target proteins is reviewed e.g. in Hoogenboom, Nat. Biotechnol. (2005) 23, 1105-1116 and Chan et al., International Immunology (2014) 26(12): 649-657, which are hereby incorporated by reference in their entirety.

The antibodies/fragments may be antagonist antibodies/fragments that inhibit or reduce a biological activity of IL-11. The antibodies/fragments may be neutralising antibodies that neutralise the biological effect of IL-11, e.g. its ability to stimulate productive signalling via an IL-11 receptor. Neutralising activity may be measured by ability to neutralise IL-11 induced proliferation in the T11 mouse plasmacytoma cell line (Nordan, R. P. et al. (1987) J. lmmunol. 139:813).

IL-11- or IL-11Rα-binding antibodies can be evaluated for the ability to antagonise IL-11-mediated signalling, e.g. using the assay described in US 2014/0219919 A1 or Blanc et al (J. Immunol Methods. 2000 Jul. 31; 241(1-2);43-59. Briefly, IL-11- and IL-11Rα-binding antibodies can be evaluated in vitro for the ability to inhibit proliferation of Ba/F3 cells expressing IL-11Rα and gp130 from the appropriate species, in response to stimulation with IL-11 from the appropriate species. Alternatively, IL-11- and IL-11Rα-binding antibodies can be analysed in vitro for the ability to inhibit the fibroblast-to-myofibroblast transition following stimulation of fibroblasts with TGFβ1, by evaluation of αSMA expression (as described e.g. in WO 2018/109174 A2 (Example 6) and WO 2018/109170 A2 (Example 6), Ng et al., Sci Transl Med. (2019) 11(511) pii: eaaw1237 and Widjaja et al., Gastroenterology (2019) 157(3):777-792).

Antibodies generally comprise six CDRs; three in the light chain variable region (VL): LC-CDR1, LC-CDR2, LC-CDR3, and three in the heavy chain variable region (VH): HC-CDR1, HC-CDR2 and HC-CDR3. The six CDRs together define the paratope of the antibody, which is the part of the antibody which binds to the target molecule. The VH region and VL region comprise framework regions (FRs) either side of each CDR, which provide a scaffold for the CDRs. From N-terminus to C-terminus, VH regions comprise the following structure: N term-[HC-FR1]-[HC-CDR1]-[HC-FR2]-[HC-CDR2]-[HC-FR3]-[HC-CDR3]-[HC-FR4]-C term; and VL regions comprise the following structure: N term-[LC-FR1]-[LC-CDR1]-[LC-FR2]-[LC-CDR2]-[LC-FR3]-[LC-CDR3]-[LC-FR4]-C term.

There are several different conventions for defining antibody CDRs and FRs, such as those described in Kabat et al., Sequences of Proteins of Immunological Interest, 5th Ed. Public Health Service, National Institutes of Health, Bethesda, Md. (1991), Chothia et al., J. Mol. Biol. 196:901-917 (1987), and VBASE2, as described in Retter et al., Nucl. Acids Res. (2005) 33 (suppl 1): D671-D674. The CDRs and FRs of the VH regions and VL regions of the antibodies described herein are defined according to the Kabat system.

In some embodiments an antibody, or an antigen-binding fragment thereof, according to the present disclosure is derived from an antibody which binds specifically to IL-11 (e.g. Enx108A, Enx203 or hEnx203). In some embodiments an antibody, or an antigen-binding fragment thereof, according to the present disclosure is derived from an antibody which binds specifically to IL-11Rα (e.g. Enx209 or hEnx209).

Antibodies and antigen-binding fragments according to the present disclosure preferably inhibit IL mediated signalling. Such antibodies/antigen-binding fragments may be described as being antagonists of IL-11-mediated signalling, and/or may be described as having the ability to neutralise IL-11-mediated signalling.

In some embodiments, the antibody/antigen-binding fragment comprises the CDRs of an antibody which binds to IL-11. In some embodiments the antibody/antigen-binding fragment comprises the CDRs of, or CDRs derived from, the CDRs of an IL-11-binding antibody described herein (e.g. Enx108A, Enx203 or hEnx203).

In some embodiments the antibody/antigen-binding fragment comprises a VH region incorporating the following CDRs:

(1)
HC-CDR1 having the amino acid sequence of SEQ ID NO:34
HC-CDR2 having the amino acid sequence of SEQ ID NO:35
HC-CDR3 having the amino acid sequence of SEQ ID NO:36,
or a variant thereof in which one or two or three amino acids in one or more of HC-CDR1, HC-CDR2, or HC-CDR3 are substituted with another amino acid.

In some embodiments the antibody/antigen-binding fragment comprises a VL region incorporating the following CDRs:

(2)
LC-CDR1 having the amino acid sequence of SEQ ID NO:37
LC-CDR2 having the amino acid sequence of SEQ ID NO:38
LC-CDR3 having the amino acid sequence of SEQ ID NO:39,
or a variant thereof in which one or two or three amino acids in one or more of LC-CDR1, LC-CDR2, or LC-CDR3 are substituted with another amino acid.

In some embodiments the antibody/antigen-binding fragment comprises a VH region incorporating the following CDRs:

(3)
HC-CDR1 having the amino acid sequence of SEQ ID NO:40
HC-CDR2 having the amino acid sequence of SEQ ID NO:41
HC-CDR3 having the amino acid sequence of SEQ ID NO:42,
or a variant thereof in which one or two or three amino acids in one or more of HC-CDR1, HC-CDR2, or HC-CDR3 are substituted with another amino acid.

In some embodiments the antibody/antigen-binding fragment comprises a VL region incorporating the following CDRs:

(4)
LC-CDR1 having the amino acid sequence of SEQ ID NO:43
LC-CDR2 having the amino acid sequence of SEQ ID NO:44
LC-CDR3 having the amino acid sequence of SEQ ID NO:45,
or a variant thereof in which one or two or three amino acids in one or more of LC-CDR1, LC-CDR2, or LC-CDR3 are substituted with another amino acid.

In some embodiments the antibody/antigen-binding fragment comprises a VH region incorporating the CDRs according to (1), and a VL region incorporating the CDRs according to (2). In some embodiments the antibody/antigen-binding fragment comprises a VH region incorporating the CDRs according to (3), and a VL region incorporating the CDRs according to (4).

In some embodiments the antibody/antigen-binding fragment comprises the VH region and the VL region of an antibody which binds to IL-11. In some embodiments the antibody/antigen-binding fragment comprises the VH region and VL region of, or a VH region and VL region derived from, the VH region and VL region of an IL-11-binding antibody described herein (e.g. Enx108A, Enx203 or hEnx203).

In some embodiments the antibody/antigen-binding fragment comprises a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:26. In some embodiments the antibody/antigen-binding fragment comprises a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:27. In some embodiments the antibody/antigen-binding fragment comprises a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100′Y° , sequence identity to the amino acid sequence of SEQ ID NO:26 and a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:27.

In some embodiments the antibody/antigen-binding fragment comprises a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:22. In some embodiments the antibody/antigen-binding fragment comprises a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100′Y° , sequence identity to the amino acid sequence of SEQ ID NO:23. In some embodiments the antibody/antigen-binding fragment comprises a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100′Y° , sequence identity to the amino acid sequence of SEQ ID NO:22 and a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:23.

In some embodiments the antibody/antigen-binding fragment comprises a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:30. In some embodiments the antibody/antigen-binding fragment comprises a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:31. In some embodiments the antibody/antigen-binding fragment comprises a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:30 and a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:31.

In some embodiments, the antibody/antigen-binding fragment comprises the CDRs of an antibody which binds to IL-11Rα. In some embodiments the antibody/antigen-binding fragment comprises the CDRs of, or CDRs derived from, the CDRs of an IL-11Rα-binding antibody described herein (e.g. Enx209 or hEnx209).

In some embodiments the antibody/antigen-binding fragment comprises a VH region incorporating the following CDRs:

(5)
HC-CDR1 having the amino acid sequence of SEQ ID NO:46
HC-CDR2 having the amino acid sequence of SEQ ID NO:47
HC-CDR3 having the amino acid sequence of SEQ ID NO:48,
or a variant thereof in which one or two or three amino acids in one or more of HC-CDR1, HC-CDR2, or HC-CDR3 are substituted with another amino acid.

In some embodiments the antibody/antigen-binding fragment comprises a VL region incorporating the following CDRs:

(6)
LC-CDR1 having the amino acid sequence of SEQ ID NO:49
LC-CDR2 having the amino acid sequence of SEQ ID NO:50
LC-CDR3 having the amino acid sequence of SEQ ID NO:51,
or a variant thereof in which one or two or three amino acids in one or more of LC-CDR1, LC-CDR2, or LC-CDR3 are substituted with another amino acid.

In some embodiments the antibody/antigen-binding fragment comprises a VH region incorporating the CDRs according to (5), and a VL region incorporating the CDRs according to (6).

In some embodiments the antibody/antigen-binding fragment comprises the VH region and the VL region of an antibody which binds to IL-11Rα. In some embodiments the antibody/antigen-binding fragment comprises the VH region and VL region of, or a VH region and VL region derived from, the VH region and VL region of an IL-11Rα-binding antibody described herein (e.g. Enx209 or hEnx209).

In embodiments in accordance with the present invention in which one or more amino acids of a reference amino acid sequence (e.g. a CDR sequence, VH region sequence or VL region sequence described herein) are substituted with another amino acid, the substitutions may conservative substitutions, for example according to the following Table. In some embodiments, amino acids in the same block in the middle column are substituted. In some embodiments, amino acids in the same line in the rightmost column are substituted:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

In some embodiments, substitution(s) may be functionally conservative. That is, in some embodiments the substitution may not affect (or may not substantially affect) one or more functional properties (e.g. target binding) of the antibody/fragment comprising the substitution relative to the equivalent unsubstituted molecule.

In some embodiments, substitution(s) relative to a reference VH region or VL region sequence may be focussed in a particular region or regions of the VH region or VL region sequence. For example, variation from a reference VH region or VL region sequence may be focussed in one or more of the framework regions (FR1, FR2, FR3 and/or FR4).

Antibodies and antigen-binding fragments according to the present disclosure may be designed and prepared using the sequences of monoclonal antibodies (mAbs) capable of binding to the relevant target molecule. Antigen-binding regions of antibodies, such as single chain variable fragment (scFv), Fab and Fab2 fragments may also be used/provided. An ‘antigen-binding region’ or ‘antigen binding fragment’ is any fragment of an antibody which is capable of binding to the target for which the given antibody is specific.

In some embodiments the antibodies/fragments comprise the VL and VH regions of an antibody which is capable of binding to IL-11, an IL-11 containing complex, or a receptor for IL-11. The VL and VH region of an antigen-binding region of an antibody together constitute the Fv region. In some embodiments the antibodies/fragments comprise or consist of the Fv region of an antibody which is capable of binding to IL-11, an IL-11 containing complex, or a receptor for IL-11. The Fv region may be expressed as a single chain wherein the VH and VL regions are covalently linked, e.g. by a flexible oligopeptide. Accordingly, antibodies/fragments may comprise or consist of an scFv comprising the VL and VH regions of an antibody which is capable of binding to IL-11, an IL-11 containing complex, or a receptor for IL-11.

The VL and light chain constant (CL) region, and the VH region and heavy chain constant 1 (CH1) region of an antigen-binding region of an antibody together constitute the Fab region. In some embodiments the antibodies/fragments comprise or consist of the Fab region of an antibody which is capable of binding to IL-11, an IL-11 containing complex, or a receptor for IL-11.

In some embodiments, antibodies/fragments comprise, or consist of, whole antibody capable of binding to IL-11, an IL-11 containing complex, or a receptor for IL-11. A “whole antibody” refers to an antibody having a structure which is substantially similar to the structure of an immunoglobulin (Ig). Different kinds of immunoglobulins and their structures are described e.g. in Schroeder and Cavacini J Allergy Clin Immunol. (2010) 125(202): S41-S52, which is hereby incorporated by reference in its entirety. Immunoglobulins of type G (i.e. IgG) are ˜150 kDa glycoproteins comprising two heavy chains and two light chains. From N- to C-terminus, the heavy chains comprise a VH followed by a heavy chain constant region comprising three constant domains (CH1, CH2, and CH3), and similarly the light chain comprises a VL followed by a CL. Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (λ).

In some embodiments the antibody/antigen-binding fragment of the present disclosure comprises an immunoglobulin heavy chain constant sequence. In some embodiments, an immunoglobulin heavy chain constant sequence may be a human immunoglobulin heavy chain constant sequence. In some embodiments the immunoglobulin heavy chain constant sequence is, or is derived from, the heavy chain constant sequence of an IgG (e.g. IgG1, IgG2, IgG3, IgG4), IgA (e.g. IgA1, IgA2), IgD, IgE or IgM, e.g. a human IgG (e.g. hIgG1, hIgG2, hIgG3, hIgG4), hIgA (e.g. hIgA1, hIgA2), hIgD, hIgE or hIgM. In some the immunoglobulin heavy chain constant sequence is, or is derived from, the heavy chain constant sequence of a human IgG1 allotype (e.g. Glm1, Glm2, Glm3 or Glm17).

In some embodiments the immunoglobulin heavy chain constant sequence is, or is derived from, the constant region sequence of human immunoglobulin G 1 constant (IGHG1; UniProt: P01857-1, v1). In some embodiments the immunoglobulin heavy chain constant sequence is, or is derived from, the constant region sequence of human immunoglobulin G 1 constant (IGHG1; UniProt: P01857-1, v1) comprising substitutions K214R, D356E and L358M (i.e. the Glm3 allotype). In some embodiments the antibody/antigen-binding fragment comprises an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 1005, sequence identity to the amino acid sequence of SEQ ID NO:52.

In some embodiments the immunoglobulin heavy chain constant sequence is, or is derived from, the constant region sequence of human immunoglobulin G 4 constant (IGHG4; UniProt: P01861, v1). In some embodiments the immunoglobulin heavy chain constant sequence is, or is derived from, the constant region sequence of human immunoglobulin G 4 constant (IGHG4; UniProt: P01861, v1) comprising substitutions S241 P and/or L248E. The S241 P mutation is hinge stabilising while the L248E mutation further reduces the already low ADCC effector function of IgG4 (Davies and Sutton, Immunol Rev. 2015 November; 268(1):139-159; Angal et al Mol Immunol. 1993 Jan;30(1):105-8). In some embodiments the antibody/antigen-binding fragment comprises an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:53.

In some embodiments the antibody/antigen-binding fragment of the present disclosure comprises an immunoglobulin light chain constant sequence. In some embodiments, an immunoglobulin light chain constant sequence may be a human immunoglobulin light chain constant sequence. In some embodiments the immunoglobulin light chain constant sequence is, or is derived from, a kappa (κ) or lambda (λ) light chain, e.g. human immunoglobulin kappa constant (IGKC; CK; UniProt: P01834-1, v2), or human immunoglobulin lambda constant (IGLC; CA), e.g. IGLC1 (UniProt: POCG04-1, v1), IGLC2 (UniProt: P0DOY2-1, v1), IGLC3 (UniProt: P0DOY3-1, v1), IGLC6 (UniProt: P0CF74-1, v1) or IGLC7 (UniProt: A0M8Q6-1, v3).

In some embodiments the antibody/antigen-binding fragment comprises an immunoglobulin light chain constant sequence. In some embodiments the immunoglobulin light chain constant sequence is, or is derived from human immunoglobulin kappa constant (IGKC; Cκ; UniProt: P01834-1, v2; SEQ ID NO:90). In some embodiments the immunoglobulin light chain constant sequence is a human immunoglobulin lambda constant (IGLC; Cλ), e.g. IGLC1, IGLC2, IGLC3, IGLC6 or IGLC7. In some embodiments the antibody/antigen-binding fragment comprises an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:54. In some embodiments the antibody/antigen-binding fragment comprises an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:55.

In some embodiments, the antibody/antigen-binding fragment comprises: (i) a polypeptide comprising or consisting of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:28, and (ii) a polypeptide comprising or consisting of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:29.

In some embodiments, the antibody/antigen-binding fragment comprises: (i) a polypeptide comprising or consisting of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:56, and (ii) a polypeptide comprising or consisting of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:57.

In some embodiments, the antibody/antigen-binding fragment comprises: (i) a polypeptide comprising or consisting of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:58, and (ii) a polypeptide comprising or consisting of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:59.

Fab, Fv, ScFv and dAb antibody fragments can all be expressed in and secreted from E. coli, thus allowing the facile production of large amounts of the said fragments.

Whole antibodies, and F(ab′)2 fragments are “bivalent”. By “bivalent” we mean that the said antibodies and F(ab′)2 fragments have two antigen combining sites. In contrast, Fab, Fv, ScFv and dAb fragments are monovalent, having only one antigen combining site. Synthetic antibodies capable of binding to IL-11, an IL-11 containing complex, or a receptor for IL-11 may also be made using phage display technology as is well known in the art.

Antibodies may be produced by a process of affinity maturation in which a modified antibody is generated that has an improvement in the affinity of the antibody for antigen, compared to an unmodified parent antibody. Affinity-matured antibodies may be produced by procedures known in the art, e.g., Marks et al., Rio/Technology 10:779-783 (1992); Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-159 (1995); and Hawkins et al, J. Mol. Biol. 226:889-896 (1992).

Antibodies/fragments include bi-specific antibodies, e.g. composed of two different fragments of two different antibodies, such that the bi-specific antibody binds two types of antigen. The bispecific antibody comprises an antibody/fragment as described herein capable of binding to IL-11, an IL-11 containing complex, or a receptor for IL-11. The antibody may contain a different fragment having affinity for a second antigen, which may be any desired antigen. Techniques for the preparation of bi-specific antibodies are well known in the art, e.g. see Mueller, D et al., (2010 Biodrugs 24 (2): 89-98), Wozniak-Knopp G et al., (2010 Protein Eng Des 23 (4): 289-297), and Baeuerle, P A et al., (2009 Cancer Res 69 (12): 4941-4944). Bispecific antibodies and bispecific antigen-binding fragments may be provided in any suitable format, such as those formats described in Kontermann MAbs 2012, 4(2): 182-197, which is hereby incorporated by reference in its entirety. For example, a bispecific antibody or bispecific antigen-binding fragment may be a bispecific antibody conjugate (e.g. an IgG2, F(ab′)2 or CovX-Body), a bispecific IgG or IgG-like molecule (e.g. an IgG, scFv4-Ig, IgG-scFv, scFv-IgG, DVD-Ig, IgG-sVD, sVD-IgG, 2 in 1-IgG, mAb2, or Tandemab common LC), an asymmetric bispecific IgG or IgG-like molecule (e.g. a kih IgG, kih IgG common LC, CrossMab, kih IgG-scFab, mAb-Fv, charge pair or SEED-body), a small bispecific antibody molecule (e.g. a Diabody (Db), dsDb, DART, scDb, tandAbs, tandem scFv (taFv), tandem dAb/VHH, triple body, triple head, Fab-scFv, or F(ab′)2-scFv2), a bispecific Fc and CH3 fusion protein (e.g. a taFv-Fc, Di-diabody, scDb-CH3, scFv-Fc-scFv, HCAb-VHH, scFv-kih-Fc, or scFv-kih-CH3), or a bispecific fusion protein (e.g. a scFv2-albumin, scDb-albumin, taFv-toxin, DNL-Fab3, DNL-Fab4-IgG, DNL-Fab4-IgG-cytokine2). See in particular FIG. 2 of Kontermann MAbs 2012, 4(2): 182-19.

Methods for producing bispecific antibodies include chemically crosslinking antibodies or antibody fragments, e.g. with reducible disulphide or non-reducible thioether bonds, for example as described in Segal and Bast, 2001. Production of Bispecific Antibodies. Current Protocols in Immunology. 14:IV:2.13:2.13.1-2.13.16, which is hereby incorporated by reference in its entirety. For example, N-succinimidyl-3-(-2-pyridyldithio)-propionate (SPDP) can be used to chemically crosslink e.g. Fab fragments via hinge region SH— groups, to create disulfide-linked bispecific F(ab)2 heterodimers.

Other methods for producing bispecific antibodies include fusing antibody-producing hybridomas e.g. with polyethylene glycol, to produce a quadroma cell capable of secreting bispecific antibody, for example as described in D. M. and Bast, B. J. 2001. Production of Bispecific Antibodies. Current Protocols in Immunology. 14:IV:2.13:2.13.1-2.13.16.

Bispecific antibodies and bispecific antigen-binding fragments can also be produced recombinantly, by expression from e.g. a nucleic acid construct encoding polypeptides for the antigen binding molecules, for example as described in Antibody Engineering: Methods and Protocols, Second Edition (Humana Press, 2012), at Chapter 40: Production of Bispecific Antibodies: Diabodies and Tandem scFv (Hornig and Farber-Schwarz), or French, How to make bispecific antibodies, Methods Mol. Med. 2000; 40:333-339.

For example, a DNA construct encoding the light and heavy chain variable domains for the two antigen binding domains (i.e. the light and heavy chain variable domains for the antigen binding domain capable of binding to IL-11, an IL-11 containing complex, or a receptor for IL-11, and the light and heavy chain variable domains for the antigen binding domain capable of binding to another target protein), and including sequences encoding a suitable linker or dimerization domain between the antigen binding domains can be prepared by molecular cloning techniques. Recombinant bispecific antibody can thereafter be produced by expression (e.g. in vitro) of the construct in a suitable host cell (e.g. a mammalian host cell), and expressed recombinant bispecific antibody can then optionally be purified.

Decoy Receptors

Peptide or polypeptide based agents capable of binding to IL-11 or IL-11 containing complexes may be based on the IL-11 receptor, e.g. an IL-11 binding fragment of an IL-11 receptor.

In some embodiments, the binding agent may comprise an IL-11-binding fragment of the IL-11Rα chain, and may preferably be soluble and/or exclude one or more, or all, of the transmembrane domain(s). In some embodiments, the binding agent may comprise an IL-11-binding fragment of gp130, and may preferably be soluble and/or exclude one or more, or all, of the transmembrane domain(s). Such molecules may be described as decoy receptors. Binding of such agents may inhibit IL-11 mediated cis and/or trans-signalling by reducing/preventing the ability of IL-11 to bind to receptors for IL-11, e.g. IL-11Rα or gp130, thereby inhibiting downstream signalling.

Curtis et al (Blood 1997 Dec. 1; 90 (11):4403-12) report that a soluble murine IL-11 receptor alpha chain (sIL-11R) was capable of antagonizing the activity of IL-11 when tested on cells expressing the transmembrane IL-11R and gp130. They proposed that the observed IL-11 antagonism by the sIL-11R depends on limiting numbers of gp130 molecules on cells already expressing the transmembrane IL-11R.

The use of soluble decoy receptors as the basis for inhibition of signal transduction and therapeutic intervention has also been reported for other signalling molecule:receptor pairs, e.g. VEGF and the VEGF receptor (De-Chao Yu et al., Molecular Therapy (2012); 20 5, 938-947; Konner and Dupont Clin Colorectal Cancer 2004 October; 4 Suppl 2:S81-5).

As such, in some embodiments a binding agent may be a decoy receptor, e.g. a soluble receptor for IL-11 and/or IL-11 containing complexes. Competition for IL-11 and/or IL-11 containing complexes provided by a decoy receptor has been reported to lead to IL-11 antagonist action (Curtis et al., supra). Decoy IL-11 receptors are also described in WO 2017/103108 A1 and WO 2018/109168 A1, which are hereby incorporated by reference in their entirety.

Decoy IL-11 receptors preferably bind IL-11 and/or IL-11 containing complexes, and thereby make these species unavailable for binding to gp130, IL-11Rα and/or gp130:1L-11Rα receptors. As such, they act as ‘decoy’ receptors for IL-11 and IL-11 containing complexes, much in the same way that etanercept acts as a decoy receptor for TNFα. IL-11-mediated signalling is reduced as compared to the level of signalling in the absence of the decoy receptor.

Decoy IL-11 receptors preferably bind to IL-11 through one or more cytokine binding modules (CBMs). The CBMs are, or are derived from or homologous to, the CBMs of naturally occurring receptor molecules for IL-11. For example, decoy IL-11 receptors may comprise, or consist of, one or more CBMs which are from, are derived from or homologous to the CBM of gp130 and/or IL-11Rα.

In some embodiments, a decoy IL-11 receptor may comprise, or consist of, an amino acid sequence corresponding to the cytokine binding module of gp130. In some embodiments, a decoy IL-11 receptor may comprise an amino acid sequence corresponding to the cytokine binding module of IL-11Rα. Herein, an amino acid sequence which ‘corresponds’ to a reference region or sequence of a given peptide/polypeptide has at least 60%, e.g. one of at least 65%, 70%, 75%, 80%, 85%, 90%, 91%, 92%, 93%, 94%, 95%, 96%, 97%, 98% or 99% sequence identity to the amino acid sequence of the reference region/sequence.

In some embodiments a decoy receptor may be able to bind IL-11, e.g. with binding affinity of at least 100 μM or less, optionally one of 10 μM or less, 1 μM or less, 100 nM or less, or about 1 to 100 nM. In some embodiments a decoy receptor may comprise all or part of the IL-11 binding domain and may optionally lack all or part of the transmembrane domains. The decoy receptor may optionally be fused to an immunoglobulin constant region, e.g. IgG Fc region.

Inhibitors

The present invention contemplates the use of inhibitor molecules capable of binding to one or more of IL-11, an IL-11 containing complex, IL-11Rα, gp130, or a complex containing IL-11Rα and/or gp130, and inhibiting IL-11 mediated signalling.

In some embodiments the agent is a peptide- or polypeptide-based binding agent based on IL-11, e.g. mutant, variant or binding fragment of IL-11. Suitable peptide or polypeptide based agents may bind to a receptor for IL-11 (e.g. IL-11Rα, gp130, or a complex containing IL-11Rα and/or gp130) in a manner that does not lead to initiation of signal transduction, or which produces sub-optimal signalling. IL-11 mutants of this kind may act as competitive inhibitors of endogenous IL-11.

For example, W147A is an IL-11 antagonist in which the amino acid 147 is mutated from a tryptophan to an alanine, which destroys the so-called ‘site III’ of IL-11. This mutant can bind to IL-11Rα, but engagement of the gp130 homodimer fails, resulting in efficient blockade of IL-11 signalling (Underhill-Day et al., 2003; Endocrinology 2003 August; 144(8):3406-14). Lee et al (Am J respire Cell Mol Biol. 2008 December; 39(6):739-746) also report the generation of an IL-11 antagonist mutant (a “mutein”) capable of specifically inhibiting the binding of IL-11 to IL-11Rα. IL-11 muteins are also described in WO 2009/052588 A1.

Menkhorst et al (Biology of Reproduction May 1, 2009 vol.80 no.5 920-927) describe a PEGylated IL-11 antagonist, PEGIL11A (CSL Limited, Parkvill, Victoria, Australia) which is effective to inhibit IL-11 action in female mice.

Pasqualini et al. Cancer (2015) 121(14):2411-2421 describe a ligand-directed, peptidomimetic drug, bone metastasis-targeting peptidomimetic-11 (BMTP-11) capable of binding to IL-11Rα.

In some embodiments a binding agent capable of binding to a receptor for IL-11 may be provided in the form of a small molecule inhibitor of one of IL-11Rα, gp130, or a complex containing IL-11Rα and/or gp130. In some embodiments a binding agent may be provided in the form of a small molecule inhibitor of IL-11 or an IL-11 containing complex, e.g. IL-11 inhibitor described in Lay et al., Int. J. Oncol. (2012); 41(2): 759-764, which is hereby incorporated by reference in its entirety.

Aptamers

In some embodiments, an agent capable of binding to IL-11/an IL-11 containing complex or a receptor for IL-11 (e.g. IL-11Rα, gp130, or a complex containing IL-11Rα and/or gp130) is an aptamer. Aptamers, also called nucleic acid/peptide ligands, are nucleic acid or peptide molecules characterised by the ability to bind to a target molecule with high specificity and high affinity. Almost every aptamer identified to date is a non-naturally occurring molecule.

Aptamers to a given target (e.g. IL-11, an IL-11 containing complex or a receptor for IL-11) may be identified and/or produced by the method of Systematic Evolution of Ligands by EXponential enrichment (SELEX™), or by developing SOMAmers (slow off-rate modified aptamers) (Gold Let al. (2010) PLoS ONE 5(12):e15004). Aptamers and SELEX are described in Tuerk and Gold, Science (1990) 249(4968):505-10, and in WO 91/19813. Applying the SELEX and the SOMAmer technology includes for instance adding functional groups that mimic amino acid side chains to expand the aptamer's chemical diversity. As a result high affinity aptamers for a target may be enriched and identified.

Aptamers may be DNA or RNA molecules and may be single stranded or double stranded. The aptamer may comprise chemically modified nucleic acids, for example in which the sugar and/or phosphate and/or base is chemically modified. Such modifications may improve the stability of the aptamer or make the aptamer more resistant to degradation and may include modification at the 2′ position of ribose.

Aptamers may be synthesised by methods which are well known to the skilled person. For example, aptamers may be chemically synthesised, e.g. on a solid support. Solid phase synthesis may use phosphoramidite chemistry. Briefly, a solid supported nucleotide is detritylated, then coupled with a suitably activated nucleoside phosphoramidite to form a phosphite triester linkage. Capping may then occur, followed by oxidation of the phosphite triester with an oxidant, typically iodine. The cycle may then be repeated to assemble the aptamer (e.g., see Sinha, N. D.; Biernat, J.; McManus, J.; Köster, H. Nucleic Acids Res. 1984, 12, 4539; and Beaucage, S. L.; Lyer, R. P. (1992). Tetrahedron 48 (12): 2223).

Suitable nucleic acid aptamers may optionally have a minimum length of one of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides. Suitable nucleic acid aptamers may optionally have a maximum length of one of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides. Suitable nucleic acid aptamers may optionally have a length of one of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides.

Aptamers may be peptides selected or engineered to bind specific target molecules. Peptide aptamers and methods for their generation and identification are reviewed in Reverdatto et al., Curr Top Med Chem. (2015) 15(12):1082-101, which is hereby incorporated by reference in its entirety. Peptide aptamers may optionally have a minimum length of one of 2, 3, 4, 5, 6, 7, 8, 9 or 10 amino acids. Peptide aptamers may optionally have a maximum length of one of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids. Suitable peptide aptamers may optionally have a length of one of 2-30, 2-25, 2-20, 5-30, 5-25 or 5-20 amino acids.

Aptamers may have K_{D}'s in the nM or pM range, e.g. less than one of 500 nM, 100 nM, 50 nM, 10 nM, 1 nM, 500 pM, 100 pM.

Properties of IL-11 Binding Agents

Agents capable of binding to IL-11/an IL-11 containing complex or a receptor for IL-11 according to the present invention may exhibit one or more of the following properties:
Specific binding to IL-11/1L-11 containing complex or a receptor for IL-11;
Binding to IL-11/IL-11 containing complex, or a receptor for IL-11, with a KD of 10 μM or less, preferably one of ≤5 μM ≤1 μM, ≤500 nM, ≤100 nM, ≤10 nM, ≤1 nM or 100 pM;
Inhibition of interaction between IL-11 and IL-11Rα;
Inhibition of interaction between IL-11 and gp130;
Inhibition of interaction between IL-11 and IL-11Rα:gp130 receptor complex;
Inhibition of interaction between IL-11:IL-11Rα complex and gp130; and
Inhibition of interaction between IL-11:IL-11Rα:gp130 complexes (i.e. multimerisation of such complexes).

These properties can be determined by analysis of the relevant agent in a suitable assay, which may involve comparison of the performance of the agent to suitable control agents. The skilled person is able to identify an appropriate control conditions for a given assay.

For example, a suitable negative control for the analysis of the ability of a test antibody/antigen-binding fragment to bind to IL-11/an IL-11 containing complex/a receptor for IL-11 may be an antibody/antigen-binding fragment directed against a non-target protein (i.e. an antibody/antigen-binding fragment which is not specific for IL-11/an IL-11 containing complex/a receptor for IL-11). A suitable positive control may be a known, validated (e.g. commercially available) IL-11- or IL-11 receptor-binding antibody. Controls may be of the same isotype as the putative IL-11/1L-11 containing complex/IL-11 receptor-binding antibody/antigen-binding fragment being analysed, and may e.g. have the same constant regions.

In some embodiments, the agent may be capable of binding specifically to IL-11 or an IL-11 containing complex, or a receptor for IL-11 (e.g. IL-11Rα, gp130, or a complex containing IL-11Rα and/or gp130). An agent which specifically binds to a given target molecule preferably binds the target with greater affinity, and/or with greater duration than it binds to other, non-target molecules.

In some embodiments the agent may bind to IL-11 or an IL-11 containing complex with greater affinity than the affinity of binding to one or more other members of the IL-6 cytokine family (e.g. IL-6, leukemia inhibitory factor (LIF), oncostatin M (OSM), cardiotrophin-1 (CT-1), ciliary neurotrophic factor (CNTF) and cardiotrophin-like cytokine (CLC)). In some embodiments the agent may bind to a receptor for IL-11 (e.g. IL-11Rα, gp130, or a complex containing IL-11Rα and/or gp130) with greater affinity than the affinity of binding to one or more other members of the IL-6 receptor family. In some embodiments the agent may bind with greater affinity to IL-11Rα than the affinity of binding to one or more of IL-6Rα, leukemia inhibitory factor receptor (LIFR), oncostatin M receptor (OSMR), ciliary neurotrophic factor receptor alpha (CNTFRα) and cytokine receptor-like factor 1 (CRLF1).

In some embodiments, the extent of binding of a binding agent to an non-target is less than about 10% of the binding of the agent to the target as measured, e.g., by ELISA, SPR, Bio-Layer Interferometry (BLI), MicroScale Thermophoresis (MST), or by a radioimmunoassay (RIA). Alternatively, the binding specificity may be reflected in terms of binding affinity, where the binding agent binds to IL-11, an IL-11 containing complex or a receptor for IL-11 with a K_{D} that is at least 0.1 order of magnitude (i.e. 0.1×10n, where n is an integer representing the order of magnitude) greater than the K_{D} towards another, non-target molecule. This may optionally be one of at least 0.2, 0.3, 0.4, 0.5, 0.6, 0.7, 0.8, 0.9, 1.0, 1.5, or 2.0.

Binding affinity for a given binding agent for its target is often described in terms of its dissociation constant (K_{D}). Binding affinity can be measured by methods known in the art, such as by ELISA, Surface Plasmon Resonance (SPR; see e.g. Hearty et al., Methods Mol Biol (2012) 907:411-442; or Rich et al., Anal Biochem. 2008 Feb. 1; 373(1):112-20), Bio-Layer Interferometry (see e.g. Lad et al., (2015) J Biomol Screen 20(4): 498-507; or Concepcion et al., Comb Chem High Throughput Screen. 2009 September; 12(8):791-800), MicroScale Thermophoresis (MST) analysis (see e.g. Jerabek-Willemsen et al., Assay Drug Dev TechnoL 2011 August; 9(4): 342-353), or by a radiolabelled antigen binding assay (RIA).

In some embodiments, the agent is capable of binding to IL-11 or an IL-11 containing complex, or a receptor for IL-11 with a K_{D} of 50 μM or less, preferably one of ≤10 μM, ≤5 pM, ≤4 μM, ≤3 μM, 2 μM, ≤1 μM, ≤500 nM, ≤100 nM, ≤75 nM, ≤50 nM, ≤40 nM, ≤30 nM, ≤20 nM, ≤15 nM, ≤12.5 nM, ≤10 nM, ≤9 nM, ≤8 nM, ≤7 nM, ≤6 nM, ≤5 nM, ≤4 nM ≤4 nM, ≤3 nM, ≤2 nM, ≤1 nM, ≤500 pM, ≤400 pM, ≤300 pM, ≤200 pM, or ≤100 pM.

In some embodiments, the agent binds to IL-11, an IL-11 containing complex or a receptor for IL-11 with an affinity of binding (e.g. as determined by ELISA) of EC50=10,000 ng/ml or less, preferably one of ≤5,000 ng/ml, ≤1000 ng/ml, ≤900 ng/ml, ≤800 ng/ml, ≤700 ng/ml, ≤600 ng/ml, ≤500 ng/ml, ≤400 ng/ml, ≤300 ng/ml, ≤200 ng/ml, ≤100 ng/ml, ≤90 ng/ml, ≤80 ng/ml, ≤70 ng/ml, ≤60 ng/ml, ≤50 ng/ml, ≤40 ng/ml, ≤30 ng/ml, ≤20 ng/ml, ≤15 ng/ml, ≤10 ng/ml, ≤7.5 ng/ml, ≤5 ng/ml, ≤2.5 ng/ml, or ≤1 ng/ml. Such ELISAs can be performed e.g. as described in Antibody Engineering, vol. 1 (2nd Edn), Springer Protocols, Springer (2010), Part V, pp657-665.

In some embodiments, the agent binds to IL-11 or an IL-11-containing complex in a region which is important for binding to a receptor for the IL-11 or IL-11-containing complex, e.g. gp130 or IL-11Rα, and thereby inhibits interaction between IL-11 or an IL-11-containing complex and a receptor for IL-11, and/or signalling through the receptor. In some embodiments, the agent binds to a receptor for IL-11 in a region which is important for binding to IL-11 or an IL-11-containing complex, and thereby inhibits interaction between IL-11 or an IL-11-containing complex and a receptor for IL-11, and/or signalling through the receptor.

The ability of a given binding agent (e.g. an agent capable of binding IL-11/an IL-11 containing complex or a receptor for IL-11) to inhibit interaction between two proteins can be determined for example by analysis of interaction in the presence of, or following incubation of one or both of the interaction partners with, the binding agent. An example of a suitable assay to determine whether a given binding agent is capable of inhibiting interaction between two interaction partners is a competition ELISA.

A binding agent which is capable of inhibiting a given interaction (e.g. between IL-11 and IL-11Rα, or between IL-11 and gp130, or between IL-11 and IL-11Rα:gp130, or between IL-11:IL-11Rα and gp130, or between IL-11:IL-11Rα:gp130 complexes) is identified by the observation of a reduction/decrease in the level of interaction between the interaction partners in the presence of—or following incubation of one or both of the interaction partners with—the binding agent, as compared to the level of interaction in the absence of the binding agent (or in the presence of an appropriate control binding agent). Suitable analysis can be performed in vitro, e.g. using recombinant interaction partners or using cells expressing the interaction partners. Cells expressing interaction partners may do so endogenously, or may do so from nucleic acid introduced into the cell. For the purposes of such assays, one or both of the interaction partners and/or the binding agent may be labelled or used in conjunction with a detectable entity for the purposes of detecting and/or measuring the level of interaction. For example, the agent may be labelled with a radioactive atom or a coloured molecule or a fluorescent molecule or a molecule which can be readily detected in any other way. Suitable detectable molecules include fluorescent proteins, luciferase, enzyme substrates, and radiolabels. The binding agent may be directly labelled with a detectable label or it may be indirectly labelled. For example, the binding agent may be unlabelled, and detected by another binding agent which is itself labelled. Alternatively, the second binding agent may have bound to it biotin and binding of labelled streptavidin to the biotin may be used to indirectly label the first binding agent.

Ability of a binding agent to inhibit interaction between two binding partners can also be determined by analysis of the downstream functional consequences of such interaction, e.g. IL-11-mediated signalling. For example, downstream functional consequences of interaction between IL-11 and IL-11Rα:gp130 or between IL-11:1L-11Rα and gp130, or between IL-11:IL-11Rα:gp130 complexes may include e.g. a process mediated by IL-11, or gene/protein expression of e.g. collagen or IL-11.

Inhibition of interaction between IL-11 or an IL-11 containing complex and a receptor for IL-11 can be analysed using 3H-thymidine incorporation and/or Ba/F3 cell proliferation assays such as those described in e.g. Curtis et al. Blood, 1997, 90(11) and Karpovich et al. Mol. Hum. Reprod. 2003 9(2): 75-80. Ba/F3 cells co-express IL-11Rα and gp130.

In some embodiments, the binding agent may be capable of inhibiting interaction between IL-11 and IL-11Rα to less than 100′Y° , e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of interaction between IL-11 and IL-11Rα in the absence of the binding agent (or in the presence of an appropriate control binding agent). In some embodiments, the binding agent may be capable of inhibiting interaction between IL-11 and IL-11Rα to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of interaction between IL-11 and IL-11Rα in the absence of the binding agent (or in the presence of an appropriate control binding agent).

In some embodiments, the binding agent may be capable of inhibiting interaction between IL-11 and gp130 to less than 100′Y° , e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of interaction between IL-11 and gp130 in the absence of the binding agent (or in the presence of an appropriate control binding agent). In some embodiments, the binding agent may be capable of inhibiting interaction between IL-11 and gp130 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of interaction between IL-11 and gp130 in the absence of the binding agent (or in the presence of an appropriate control binding agent).

In some embodiments, the binding agent may be capable of inhibiting interaction between IL-11 and IL-11Rα:gp130 to less than 100%, e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of interaction between IL-11 and IL-11Rα:gp130 in the absence of the binding agent (or in the presence of an appropriate control binding agent). In some embodiments, the binding agent may be capable of inhibiting interaction between IL-11 and IL-11Rα:gp130 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of interaction between IL-11 and IL-11Rα:gp130 in the absence of the binding agent (or in the presence of an appropriate control binding agent).

In some embodiments, the binding agent may be capable of inhibiting interaction between IL-11:1L-11Rα complex and gp130 to less than 100%, e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of interaction between IL-11:IL-11Rα complex and gp130 in the absence of the binding agent (or in the presence of an appropriate control binding agent). In some embodiments, the binding agent is capable of inhibiting interaction between IL-11:IL-11Rα complex and gp130 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of interaction between IL-11:IL-11Rα complex and gp130 in the absence of the binding agent.

In some embodiments, the binding agent may be capable of inhibiting interaction between IL-11:IL-11Rα:gp130 complexes (i.e. multimerisation of such complexes) to less than 100%, e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of interaction between IL-11:IL-11Rα:gp130 complexes in the absence of the binding agent (or in the presence of an appropriate control binding agent). In some embodiments, the binding agent is capable of inhibiting interaction between IL-11:IL-11Rα:gp130 complexes to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of interaction between IL-11:IL-11Rα:gp130 complexes in the absence of the binding agent.

Agents Capable of Reducing Expression of IL-11 or an IL-11 Receptor

In aspects of the present invention the agent capable of inhibiting IL-11-mediated signalling may be capable of preventing or reducing the expression of one or more of IL-11, IL-11Rα or gp130.

Expression may be gene or protein expression, and may be determined as described herein or by methods in the art that will be well known to a skilled person. Expression may be by a cell/tissue/organ/organ system of a subject.

Suitable agents may be of any kind, but in some embodiments an agent capable of preventing or reducing the expression of one or more of IL-11, IL-11Rα or gp130 may be a small molecule or an oligonucleotide.

An agent capable of preventing or reducing of the expression of one or more of IL-11, IL-11Rα or gp130 may do so e.g. through inhibiting transcription of the gene encoding IL-11, IL-11Rα or gp130, inhibiting post-transcriptional processing of RNA encoding IL-11, IL-11Rα or gp130, reducing the stability of RNA encoding IL-11, IL-11Rα or gp130, promoting degradation of RNA encoding IL-11, IL-11Rα or gp130, inhibiting post-translational processing of IL-11, IL-11Rα or gp130 polypeptide, reducing the stability of IL-11, IL-11Rα or gp130 polypeptide or promoting degradation of IL-11, IL-11Rα or gp130 polypeptide.

Taki et al. Clin Exp Immunol (1998) April; 112(1): 133-138 reported a reduction in the expression of IL-11 in rheumatoid synovial cells upon treatment with indomethacin, dexamethasone or interferon-gamma (IFNγ).

The present invention contemplates the use of antisense nucleic acid to prevent/reduce expression of IL-11, IL-11Rα or gp130. In some embodiments, an agent capable of preventing or reducing the expression of IL-11, IL-11Rα or gp130 may cause reduced expression by RNA interference (RNAi).

In some embodiments, the agent may be an inhibitory nucleic acid, such as antisense or small interfering RNA, including but not limited to shRNA or siRNA.

In some embodiments the inhibitory nucleic acid is provided in a vector. For example, in some embodiments the agent may be a lentiviral vector encoding shRNA for one or more of IL-11, IL-11Rα or gp130.

Oligonucleotide molecules, particularly RNA, may be employed to regulate gene expression. These include antisense oligonucleotides, targeted degradation of mRNAs by small interfering RNAs (siRNAs), post transcriptional gene silencing (PTGs), developmentally regulated sequence-specific translational repression of mRNA by micro-RNAs (miRNAs) and targeted transcriptional gene silencing.

An antisense oligonucleotide is an oligonucleotide, preferably single-stranded, that targets and binds, by complementary sequence binding, to a target oligonucleotide, e.g. mRNA. Where the target oligonucleotide is an mRNA, binding of the antisense to the mRNA blocks translation of the mRNA and expression of the gene product. Antisense oligonucleotides may be designed to bind sense genomic nucleic acid and inhibit transcription of a target nucleotide sequence.

In view of the known nucleic acid sequences for IL-11, IL-11Rα and gp130 (e.g. the known mRNA sequences available from GenBank under Accession No.s: BC012506.1 GI:15341754 (human IL-11), BC134354.1 GI:126632002 (mouse IL-11), AF347935.1 GI:13549072 (rat IL-11), NM_001142784.2 GI:391353394 (human IL-11Rα), NM_001163401.1 GI:254281268 (mouse IL-11Rα), NM_139116.1 GI:20806172 (rat IL-11Rα), NM_001190981.1 GI:300244534 (human gp130), NM_010560.3 GI:225007624 (mouse gp130), NM_001008725.3 GI:300244570 (rat gp130)) oligonucleotides may be designed to repress or silence the expression of IL-11, IL-11Rα or gp130.

Such oligonucleotides may have any length, but may preferably be short, e.g. less than 100 nucleotides, e.g. 10-40 nucleotides, or 20-50 nucleotides, and may comprise a nucleotide sequence having complete- or near-complementarity (e.g. 80%, 85%, 90%, 91%, 92%, 93%, 94%, 95%, 96%, 97%, 98%, 99% or 100% complementarity) to a sequence of nucleotides of corresponding length in the target oligonucleotide, e.g. the IL-11, IL-11Rα or gp130 mRNA. The complementary region of the nucleotide sequence may have any length, but is preferably at least 5, and optionally no more than 50, nucleotides long, e.g. one of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

Repression of expression of IL-11, IL-11Rα or gp130 will preferably result in a decrease in the quantity of IL-11, IL-11Rα or gp130 expressed by a cell/tissue/organ/organ system/subject. For example, in a given cell the repression of IL-11, IL-11Rα or gp130 by administration of a suitable nucleic acid will result in a decrease in the quantity of IL-11, IL-11Rα or gp130 expressed by that cell relative to an untreated cell. Repression may be partial. Preferred degrees of repression are at least 50%, more preferably one of at least 60%, 70%, 80%, 85% or 90%. A level of repression between 90% and 100% is considered a ‘silencing’ of expression or function.

A role for the RNAi machinery and small RNAs in targeting of heterochromatin complexes and epigenetic gene silencing at specific chromosomal loci has been demonstrated. Double-stranded RNA (dsRNA)-dependent post transcriptional silencing, also known as RNA interference (RNAi), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response. The decrease in expression of targeted gene products can be extensive with 90% silencing induced by a few molecules of siRNA. RNAi based therapeutics have been progressed into Phase I, II and III clinical trials for a number of indications (Nature 2009 Jan. 22; 457(7228):426-433).

In the art, these RNA sequences are termed “short or small interfering RNAs” (siRNAs) or “microRNAs” (miRNAs) depending on their origin. Both types of sequence may be used to down-regulate gene expression by binding to complementary RNAs and either triggering mRNA elimination (RNAi) or arresting mRNA translation into protein. siRNA are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

siRNA ligands are typically double stranded and, in order to optimise the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNA ligands are typically single stranded and have regions that are partially complementary enabling the ligands to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is discussed in John et al, PLoS Biology, 11(2), 1862-1879, 2004.

Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double-stranded siRNA, the molecule may have symmetric 3′ overhangs, e.g. of one or two (ribo)nucleotides, typically a UU of dTdT 3′ overhang. Based on the disclosure provided herein, the skilled person can readily design suitable siRNA and miRNA sequences, for example using resources such the Ambion siRNA finder. siRNA and miRNA sequences can be synthetically produced and added exogenously to cause gene downregulation or produced using expression systems (e.g. vectors). In a preferred embodiment the siRNA is synthesized synthetically.

Longer double stranded RNAs may be processed in the cell to produce siRNAs (see for example Myers (2003) Nature Biotechnology 21:324-328). The longer dsRNA molecule may have symmetric 3′ or 5′ overhangs, e.g. of one or two (ribo)nucleotides, or may have blunt ends. The longer dsRNA molecules may be 25 nucleotides or longer. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length. dsRNAs 30 nucleotides or more in length may be expressed using the vector pDECAP (Shinagawa et al., Genes and Dev., 17, 1340-5, 2003).

Another alternative is the expression of a short hairpin RNA molecule (shRNA) in the cell. shRNAs are more stable than synthetic siRNAs. A shRNA consists of short inverted repeats separated by a small loop sequence. One inverted repeat is complimentary to the gene target. In the cell the shRNA is processed by DICER into a siRNA which degrades the target gene mRNA and suppresses expression. In a preferred embodiment the shRNA is produced endogenously (within a cell) by transcription from a vector. shRNAs may be produced within a cell by transfecting the cell with a vector encoding the shRNA sequence under control of a RNA polymerase III promoter such as the human H1 or 7SK promoter or a RNA polymerase II promoter. Alternatively, the shRNA may be synthesised exogenously (in vitro) by transcription from a vector. The shRNA may then be introduced directly into the cell. Preferably, the shRNA molecule comprises a partial sequence of IL-11, IL-11Rα or gp130. Preferably, the shRNA sequence is between 40 and 100 bases in length, more preferably between 40 and 70 bases in length. The stem of the hairpin is preferably between 19 and 30 base pairs in length. The stem may contain G-U pairings to stabilise the hairpin structure.

siRNA molecules, longer dsRNA molecules or miRNA molecules may be made recombinantly by transcription of a nucleic acid sequence, preferably contained within a vector. Preferably, the siRNA molecule, longer dsRNA molecule or miRNA molecule comprises a partial sequence of IL-11, IL-11Rα or gp130.

In one embodiment, the siRNA, longer dsRNA or miRNA is produced endogenously (within a cell) by transcription from a vector. The vector may be introduced into the cell in any of the ways known in the art. Optionally, expression of the RNA sequence can be regulated using a tissue specific (e.g. heart, liver, or kidney specific) promoter. In a further embodiment, the siRNA, longer dsRNA or miRNA is produced exogenously (in vitro) by transcription from a vector.

Suitable vectors may be oligonucleotide vectors configured to express the oligonucleotide agent capable of IL-11, IL-11Rα or gp130 repression. Such vectors may be viral vectors or plasmid vectors. The therapeutic oligonucleotide may be incorporated in the genome of a viral vector and be operably linked to a regulatory sequence, e.g. promoter, which drives its expression. The term “operably linked” may include the situation where a selected nucleotide sequence and regulatory nucleotide sequence are covalently linked in such a way as to place the expression of a nucleotide sequence under the influence or control of the regulatory sequence. Thus a regulatory sequence is operably linked to a selected nucleotide sequence if the regulatory sequence is capable of effecting transcription of a nucleotide sequence which forms part or all of the selected nucleotide sequence.

Viral vectors encoding promoter-expressed siRNA sequences are known in the art and have the benefit of long term expression of the therapeutic oligonucleotide. Examples include lentiviral (Nature 2009 Jan. 22; 457(7228):426-433), adenovirus (Shen et al., FEBS Lett 2003 Mar. 27; 539(1-3)111-4) and retroviruses (Barton and Medzhitov PNAS Nov. 12, 2002 vol. 99, no. 23 14943-14945).

In other embodiments a vector may be configured to assist delivery of the therapeutic oligonucleotide to the site at which repression of IL-11, IL-11Rα or gp130 expression is required. Such vectors typically involve complexing the oligonucleotide with a positively charged vector (e.g., cationic cell penetrating peptides, cationic polymers and dendrimers, and cationic lipids); conjugating the oligonucleotide with small molecules (e.g., cholesterol, bile acids, and lipids), polymers, antibodies, and RNAs; or encapsulating the oligonucleotide in nanoparticulate formulations (Wang et al., AAPS J. 2010 December; 12(4): 492-503).

In one embodiment, a vector may comprise a nucleic acid sequence in both the sense and antisense orientation, such that when expressed as RNA the sense and antisense sections will associate to form a double stranded RNA.

Alternatively, siRNA molecules may be synthesized using standard solid or solution phase synthesis techniques which are known in the art. Linkages between nucleotides may be phosphodiester bonds or alternatives, for example, linking groups of the formula P(O)S, (thioate); P(S)S, (dithioate); P(O)NR′2; P(O)R′; P(O)0R6; CO; or CONR′2 wherein R is H (or a salt) or alkyl (1-12C) and R6 is alkyl (1-9C) is joined to adjacent nucleotides through —O— or —S—.

Modified nucleotide bases can be used in addition to the naturally occurring bases, and may confer advantageous properties on siRNA molecules containing them.

For example, modified bases may increase the stability of the siRNA molecule, thereby reducing the amount required for silencing. The provision of modified bases may also provide siRNA molecules which are more, or less, stable than unmodified siRNA.

The term ‘modified nucleotide base’ encompasses nucleotides with a covalently modified base and/or sugar. For example, modified nucleotides include nucleotides having sugars which are covalently attached to low molecular weight organic groups other than a hydroxyl group at the 3′position and other than a phosphate group at the 5′position. Thus modified nucleotides may also include 2′substituted sugars such as 2′-O-methyl-; 2′-O-alkyl; 2′-O-allyl; 2′-S-alkyl; 2′-S-allyl; 2′-fluoro-; 2′-halo or azido-ribose, carbocyclic sugar analogues, α-anomeric sugars; epimeric sugars such as arabinose, xyloses or lyxoses, pyranose sugars, furanose sugars, and sedoheptulose.

Modified nucleotides are known in the art and include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N6-methyladenine, 4-acetylcytosine,5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2-methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5methoxyuracil, 2 methylthio-N6-isopentenyladenine, uracil-5-oxyacetic acid methyl ester, pseudouracil, 2-thiocytosine, 5-methyl-2 thiouracil, 2-thiouracil, 4-thiouracil, 5methyluracil, N-uracil-5-oxyacetic acid methylester, uracil 5-oxyacetic acid, queosine, 2-thiocytosine, 5-propyluracil, 5-propylcytosine, 5-ethyluracil, 5ethylcytosine, 5-butyluracil, 5-pentyluracil, 5-pentylcytosine, and 2,6,diaminopurine, methylpsuedouracil, 1-methylguanine, 1-methylcytosine.

Methods relating to the use of RNAi to silence genes in C. elegans, Drosophila, plants, and mammals are known in the art (Fire A, et al., 1998 Nature 391:806-811; Fire, A. Trends Genet. 15, 358-363 (1999); Sharp, P. A. RNA interference 2001. Genes Dev. 15, 485-490 (2001); Hammond, S. M., et al., Nature Rev. Genet. 2, 110-1119 (2001); Tuschl, T. Chem. Biochem. 2, 239-245 (2001); Hamilton, A. et al., Science 286, 950-952 (1999); Hammond, S. M., et al., Nature 404, 293-296 (2000); Zamore, P. D., et al., Cell 101, 25-33 (2000); Bernstein, E., et al., Nature 409, 363-366 (2001); Elbashir, S. M., et al., Genes Dev. 15, 188-200 (2001); WO0129058; WO9932619, and Elbashir S M, et al., 2001 Nature 411:494-498).

Accordingly, the invention provides nucleic acid that is capable, when suitably introduced into or expressed within a mammalian, e.g. human, cell that otherwise expresses IL-11, IL-11Rα or gp130, of suppressing IL-11, IL-11Rα or gp130 expression by RNAi.

Nucleic acid sequences for IL-11, IL-11Rα and gp130 (e.g. the known mRNA sequences available from GenBank under Accession No.s: BC012506.1 GI:15341754 (human IL-11), BC134354.1 GI:126632002 (mouse IL-11), AF347935.1 GI:13549072 (rat IL-11), NM_001142784.2 GI:391353394 (human IL-11Rα), NM_001163401.1 GI:254281268 (mouse IL-11Rα), NM_139116.1 GI:20806172 (rat IL-11Rα), NM_001190981.1 GI:300244534 (human gp130), NM_010560.3 GI:225007624 (mouse gp130), NM_001008725.3 GI:300244570 (rat gp130)) oligonucleotides may be designed to repress or silence the expression of IL-11, IL-11Rα or gp130.

The nucleic acid may have substantial sequence identity to a portion of IL-11, IL-11Rα or gp130 mRNA, e.g. as defined in GenBank accession no. NM_000641.3 GI:391353405 (IL-11), NM_001142784.2 GI:391353394 (IL-11Ra), NM_001190981.1 GI:300244534 (gp130) or the complementary sequence to said mRNA.

The nucleic acid may be a double-stranded siRNA. (As the skilled person will appreciate, and as explained further below, a siRNA molecule may include a short 3′ DNA sequence also.)

Alternatively, the nucleic acid may be a DNA (usually double-stranded DNA) which, when transcribed in a mammalian cell, yields an RNA having two complementary portions joined via a spacer, such that the RNA takes the form of a hairpin when the complementary portions hybridise with each other. In a mammalian cell, the hairpin structure may be cleaved from the molecule by the enzyme DICER, to yield two distinct, but hybridised, RNA molecules.

In some preferred embodiments, the nucleic acid is generally targeted to the sequence of one of SEQ ID NOs 4 to 7 (IL-11) or to one of SEQ ID NOs 8 to 11 (IL-11Rα).

Only single-stranded (i.e. non self-hybridised) regions of an mRNA transcript are expected to be suitable targets for RNAi. It is therefore proposed that other sequences very close in the IL-11 or IL-11Rα mRNA transcript to the sequence represented by one of SEQ ID NOs 4 to 7 or 8 to 11 may also be suitable targets for RNAi. Such target sequences are preferably 17-23 nucleotides in length and preferably overlap one of SEQ ID NOs 4 to 7 or 8 to 11 by at least 1, 2, 3, 4, 5, 6, 7, 8, 9, 10, 11, 12, 13, 14, 15, 16, 17, 18 or all 19 nucleotides (at either end of one of SEQ ID NOs 4 to 7 or 8 to 11).

Accordingly, the invention provides nucleic acid that is capable, when suitably introduced into or expressed within a mammalian cell that otherwise expresses IL-11 or IL-11Rα, of suppressing IL-11 or IL-11Rα expression by RNAi, wherein the nucleic acid is generally targeted to the sequence of one of SEQ ID NOs 4 to 7 or 8 to 11.

By “generally targeted” the nucleic acid may target a sequence that overlaps with SEQ ID NOs 4 to 7 or 8 to 11. In particular, the nucleic acid may target a sequence in the mRNA of human IL-11 or IL-11Rα that is slightly longer or shorter than one of SEQ ID NOs 4 to 7 or 8 to 11 (preferably from 17-23 nucleotides in length), but is otherwise identical to one of SEQ ID NOs 4 to 7 or 8 to 11.

It is expected that perfect identity/complementarity between the nucleic acid of the invention and the target sequence, although preferred, is not essential. Accordingly, the nucleic acid of the invention may include a single mismatch compared to the mRNA of IL-11 or IL-11Rα. It is expected, however, that the presence of even a single mismatch is likely to lead to reduced efficiency, so the absence of mismatches is preferred. When present, 3′ overhangs may be excluded from the consideration of the number of mismatches.

The term “complementarity” is not limited to conventional base pairing between nucleic acid consisting of naturally occurring ribo- and/or deoxyribonucleotides, but also includes base pairing between mRNA and nucleic acids of the invention that include non-natural nucleotides.

In one embodiment, the nucleic acid (herein referred to as double-stranded siRNA) includes the double-stranded RNA sequences shown in SEQ ID NOs 12 to 15. In another embodiment, the nucleic acid (herein referred to as double-stranded siRNA) includes the double-stranded RNA sequences shown in SEQ ID NOs 16 to 19.

However, it is also expected that slightly shorter or longer sequences directed to the same region of IL-11 or IL-11Rα mRNA will also be effective. In particular, it is expected that double-stranded sequences between 17 and 23 bp in length will also be effective.

The strands that form the double-stranded RNA may have short 3′ dinucleotide overhangs, which may be DNA or RNA. The use of a 3′ DNA overhang has no effect on siRNA activity compared to a 3′ RNA overhang, but reduces the cost of chemical synthesis of the nucleic acid strands (Elbashir et al., 2001c). For this reason, DNA dinucleotides may be preferred.

When present, the dinucleotide overhangs may be symmetrical to each other, though this is not essential. Indeed, the 3′ overhang of the sense (upper) strand is irrelevant for RNAi activity, as it does not participate in mRNA recognition and degradation (Elbashir et al., 2001a, 2001b, 2001c).

While RNAi experiments in Drosophila show that antisense 3′ overhangs may participate in mRNA recognition and targeting (Elbashir et al. 2001c), 3′ overhangs do not appear to be necessary for RNAi activity of siRNA in mammalian cells. Incorrect annealing of 3′ overhangs is therefore thought to have little effect in mammalian cells (Elbashir et al. 2001c; Czauderna et al. 2003).

Any dinucleotide overhang may therefore be used in the antisense strand of the siRNA. Nevertheless, the dinucleotide is preferably -UU or -UG (or -TT or -TG if the overhang is DNA), more preferably -UU (or -TT). The -UU (or -TT) dinucleotide overhang is most effective and is consistent with (i.e. capable of forming part of) the RNA polymerase III end of transcription signal (the terminator signal is TTTTT). Accordingly, this dinucleotide is most preferred. The dinucleotides AA, CC and GG may also be used, but are less effective and consequently less preferred.

Moreover, the 3′ overhangs may be omitted entirely from the siRNA.

The invention also provides single-stranded nucleic acids (herein referred to as single-stranded siRNAs) respectively consisting of a component strand of one of the aforementioned double-stranded nucleic acids, preferably with the 3′-overhangs, but optionally without. The invention also provides kits containing pairs of such single-stranded nucleic acids, which are capable of hybridising with each other in vitro to form the aforementioned double-stranded siRNAs, which may then be introduced into cells.

The invention also provides DNA that, when transcribed in a mammalian cell, yields an RNA (herein also referred to as an shRNA) having two complementary portions which are capable of self-hybridising to produce a double-stranded motif, e.g. including a sequence selected from the group consisting of SEQ ID NOs: 12 to 15 or 16 to 19 or a sequence that differs from any one of the aforementioned sequences by a single base pair substitution.

The complementary portions will generally be joined by a spacer, which has suitable length and sequence to allow the two complementary portions to hybridise with each other. The two complementary (i.e. sense and antisense) portions may be joined 5′-3′ in either order. The spacer will typically be a short sequence, of approximately 4-12 nucleotides, preferably 4-9 nucleotides, more preferably 6-9 nucleotides.

Preferably the 5′ end of the spacer (immediately 3′ of the upstream complementary portion) consists of the nucleotides -UU- or -UG-, again preferably -UU- (though, again, the use of these particular dinucleotides is not essential). A suitable spacer, recommended for use in the pSuper system of OligoEngine (Seattle, Wash., USA) is UUCAAGAGA. In this and other cases, the ends of the spacer may hybridise with each other, e.g. elongating the double-stranded motif beyond the exact sequences of SEQ ID NOs 12 to 15 or 16 to 19 by a small number (e.g. 1 or 2) of base pairs.

Similarly, the transcribed RNA preferably includes a 3′ overhang from the downstream complementary portion. Again, this is preferably -UU or -UG, more preferably -UU.

Such shRNA molecules may then be cleaved in the mammalian cell by the enzyme DICER to yield a double-stranded siRNA as described above, in which one or each strand of the hybridised dsRNA includes a 3′ overhang.

Techniques for the synthesis of the nucleic acids of the invention are of course well known in the art.

The skilled person is well able to construct suitable transcription vectors for the DNA of the invention using well-known techniques and commercially available materials. In particular, the DNA will be associated with control sequences, including a promoter and a transcription termination sequence.

Of particular suitability are the commercially available pSuper and pSuperior systems of OligoEngine (Seattle, Wash., USA). These use a polymerase-III promoter (H1) and a T5 transcription terminator sequence that contributes two U residues at the 3′ end of the transcript (which, after DICER processing, provide a 3′ UU overhang of one strand of the siRNA).

Another suitable system is described in Shin et al. (RNA, 2009 May; 15(5): 898-910), which uses another polymerase-III promoter (U6).

The double-stranded siRNAs of the invention may be introduced into mammalian cells in vitro or in vivo using known techniques, as described below, to suppress expression of IL-11 or a receptor for IL-11.

Similarly, transcription vectors containing the DNAs of the invention may be introduced into tumour cells in vitro or in vivo using known techniques, as described below, for transient or stable expression of RNA, again to suppress expression of IL-11 or a receptor for IL-11.

Accordingly, the invention also provides a method of suppressing expression of IL-11 or a receptor for IL-11 in a mammalian, e.g. human, cell, the method comprising administering to the cell a double-stranded siRNA of the invention or a transcription vector of the invention.

Similarly, the invention further provides a method of treating diseases/conditions characterised by type IV collagen dysfunction, comprising administering to a subject a double-stranded siRNA of the invention or a transcription vector of the invention.

The invention further provides the double-stranded siRNAs of the invention and the transcription vectors of the invention, for use in a method of treatment, preferably a method of treating a disease/condition characterised by type IV collagen dysfunction.

The invention further provides the use of the double-stranded siRNAs of the invention and the transcription vectors of the invention in the preparation of a medicament for the treatment of a disease/condition characterised by type IV collagen dysfunction.

The invention further provides a composition comprising a double-stranded siRNA of the invention or a transcription vector of the invention in admixture with one or more pharmaceutically acceptable carriers. Suitable carriers include lipophilic carriers or vesicles, which may assist in penetration of the cell membrane.

Materials and methods suitable for the administration of siRNA duplexes and DNA vectors of the invention are well known in the art and improved methods are under development, given the potential of RNAi technology.

Generally, many techniques are available for introducing nucleic acids into mammalian cells. The choice of technique will depend on whether the nucleic acid is transferred into cultured cells in vitro or in vivo in the cells of a patient. Techniques suitable for the transfer of nucleic acid into mammalian cells in vitro include the use of liposomes, electroporation, microinjection, cell fusion, DEAE, dextran and calcium phosphate precipitation. In vivo gene transfer techniques include transfection with viral (typically retroviral) vectors and viral coat protein-liposome mediated transfection (Dzau et al. (2003) Trends in Biotechnology 11, 205-210).

In particular, suitable techniques for cellular administration of the nucleic acids of the invention both in vitro and in vivo are disclosed in the following articles:

General reviews: Borkhardt, A. 2002. Blocking oncogenes in malignant cells by RNA interference—new hope for a highly specific cancer treatment? Cancer Cell. 2:167-8. Hannon, G. J. 2002. RNA interference. Nature. 418:244-51. McManus, M. T., and P. A. Sharp. 2002. Gene silencing in mammals by small interfering RNAs. Nat Rev Genet. 3:737-47. Scherr, M., M. A. Morgan, and M. Eder. 2003b. Gene silencing mediated by small interfering RNAs in mammalian cells. Curr Med Chem. 10:245-56. Shuey, D. J., D. E. McCallus, and T. Giordano. 2002. RNAi: gene-silencing in therapeutic intervention. Drug Discov Today. 7:1040-6.

Systemic delivery using liposomes: Lewis, D. L., J. E. Hagstrom, A. G. Loomis, J. A. Wolff, and H. Herweijer. 2002. Efficient delivery of siRNA for inhibition of gene expression in postnatal mice. Nat Genet. 32:107-8. Paul, C. P., P. D. Good, I. Winer, and D. R. Engelke. 2002. Effective expression of small interfering RNA in human cells. Nat Biotechnol. 20:505-8. Song, E., S. K. Lee, J. Wang, N. Ince, N. Ouyang, J. Min, J. Chen, P. Shankar, and J. Lieberman. 2003. RNA interference targeting Fas protects mice from fulminant hepatitis. Nat Med. 9:347-51. Sorensen, D.R., M. Leirdal, and M. Sioud. 2003. Gene silencing by systemic delivery of synthetic siRNAs in adult mice. J Mol Biol. 327:761-6.

Virus mediated transfer: Abbas-Terki, T., W. Blanco-Bose, N. Deglon, W. Pralong, and P. Aebischer. 2002. Lentiviral-mediated RNA interference. Hum Gene Ther. 13:2197-201. Barton, G. M., and R. Medzhitov. 2002. Retroviral delivery of small interfering RNA into primary cells. Proc Natl Acad Sci USA. 99:14943-5. Devroe, E., and P. A. Silver. 2002. Retrovirus-delivered siRNA. BMC Biotechnol. 2:15. Lori, F., P. Guallini, L. Galluzzi, and J. Lisziewicz. 2002. Gene therapy approaches to HIV infection. Am J Pharmacogenomics. 2:245-52. Matta, H., B. Hozayev, R. Tomar, P. Chugh, and P. M. Chaudhary. 2003. Use of lentiviral vectors for delivery of small interfering RNA. Cancer Biol Ther. 2:206-10. Qin, X. F., D. S. An, I. S. Chen, and D. Baltimore. 2003. Inhibiting HIV-1 infection in human T cells by lentiviral-mediated delivery of small interfering RNA against CCR5. Proc Natl Acad Sci USA. 100:183-8. Scherr, M., K. Battmer, A. Ganser, and M. Eder. 2003a. Modulation of gene expression by lentiviral-mediated delivery of small interfering RNA. Cell Cycle. 2:251-7. Shen, C., A.K. Buck, X. Liu, M. Winkler, and S. N. Reske. 2003. Gene silencing by adenovirus-delivered siRNA. FEBS Lett. 539:111-4.

Peptide delivery: Morris, M. C., L. Chaloin, F. Heitz, and G. Divita. 2000. Translocating peptides and proteins and their use for gene delivery. Curr Opin Biotechnol. 11:461-6. Simeoni, F., M. C. Morris, F. Heitz, and G. Divita. 2003. Insight into the mechanism of the peptide-based gene delivery system MPG: implications for delivery of siRNA into mammalian cells. Nucleic Acids Res. 31:2717-24. Other technologies that may be suitable for delivery of siRNA to the target cells are based on nanoparticles or nanocapsules such as those described in U.S. Pat. Nos. 6,649,192B and 5,843,509B.

Inhibition of IL-11-Mediated Signalling

In embodiments of the present invention, agents capable of inhibiting the action of IL-11 may possess one or more of the following functional properties:
Inhibition of signalling mediated by IL-11;
Inhibition of signalling mediated by binding of IL-11 to IL-11Rα:gp130 receptor complex;
Inhibition of signalling mediated by binding of IL-11:IL-11Rα complex to gp130 (i.e. IL-11 trans signalling);
Inhibition of signalling mediated by multimerisation of IL-11:IL-11Rα:gp130 complexes;
Inhibition of a process mediated by IL-11;
Inhibition of gene/protein expression of IL-11 and/or IL-11Rα.

These properties can be determined by analysis of the relevant agent in a suitable assay, which may involve comparison of the performance of the agent to suitable control agents. The skilled person is able to identify an appropriate control conditions for a given assay.

IL-11-mediated signalling and/or processes mediated by IL-11 includes signalling mediated by fragments of IL-11 and polypeptide complexes comprising IL-11 or fragments thereof. IL-11-mediated signalling may be signalling mediated by human IL-11 and/or mouse IL-11. Signalling mediated by IL-11 may occur following binding of IL-11 or an IL-11 containing complex to a receptor to which IL-11 or said complex binds.

In some embodiments, an agent may be capable of inhibiting the biological activity of IL-11 or an IL-11-containing complex.

In some embodiments, the agent is an antagonist of one or more signalling pathways which are activated by signal transduction through receptors comprising IL-11Rα and/or gp130, e.g. IL-11Rα:gp130. In some embodiments, the agent is capable of inhibiting signalling through one or more immune receptor complexes comprising IL-11Rα and/or gp130, e.g. IL-11Rα:gp130. In various aspects of the present invention, an agent provided herein is capable of inhibiting IL-11-mediated cis and/or trans signalling. In some embodiments in accordance with the various aspects of the present invention an agent provided herein is capable of inhibiting IL-11-mediated cis signalling.

In some embodiments, the agent may be capable of inhibiting IL-11-mediated signalling to less than 100%, e.g. one of 99% or less, 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of signalling in the absence of the agent (or in the presence of an appropriate control agent). In some embodiments, the agent is capable of reducing IL-11-mediated signalling to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of signalling in the absence of the agent (or in the presence of an appropriate control agent).

In some embodiments, the IL-11-mediated signalling may be signalling mediated by binding of IL-11 to IL-11Rα:gp130 receptor. Such signalling can be analysed e.g. by treating cells expressing IL-11Rα and gp130 with IL-11, or by stimulating IL-11 production in cells which express IL-11Rα and gp130.

The IC₅₀ for an agent for inhibition of IL-11-mediated signalling may be determined, e.g. by culturing Ba/F3 cells expressing IL-11Rα and gp130 in the presence of human IL-11 and the agent, and measuring 3H-thymidine incorporation into DNA. In some embodiments, the agent may exhibit an IC₅₀ of 10 μg/ml or less, preferably one of 5 μg/ml, 4 μg/ml, 3.5 μg/ml, 3 μg/ml, 2 μg/ml, 1 μg/ml, 0.9 μg/ml, 0.8 μg/ml, 0.7 μg/ml, 0.6 μg/ml, or 0.5 μg/ml in such an assay.

In some embodiments, the IL-11-mediated signalling may be signalling mediated by binding of IL-11:IL-11Rα complex to gp130. In some embodiments, the IL-11:1L-11Rα complex may be soluble, e.g. complex of extracellular domain of IL-11Rα and IL-11, or complex of soluble IL-11Rα isoform/fragment and IL-11. In some embodiments, the soluble IL-11Rα is a soluble (secreted) isoform of IL-11Rα, or is the liberated product of proteolytic cleavage of the extracellular domain of cell membrane bound IL-11Rα.

In some embodiments, the IL-11:1L-11Rα complex may be cell-bound, e.g. complex of cell-membrane bound IL-11Rα and IL-11. Signalling mediated by binding of IL-11:1L-11Rα complex to gp130 can be analysed by treating cells expressing gp130 with IL-11:1L-11Rα complex, e.g. recombinant fusion protein comprising IL-11 joined by a peptide linker to the extracellular domain of IL-11Rα, e.g. hyper IL-11. Hyper IL-11 was constructed using fragments of IL-11Rα (amino acid residues 1 to 317 consisting of domain 1 to 3; UniProtKB: Q14626) and IL-11 (amino acid residues 22 to 199 of UniProtKB: P20809) with a 20 amino acid long linker (SEQ ID NO:20). The amino acid sequence for Hyper IL-11 is shown in SEQ ID NO:21.

In some embodiments, the agent may be capable of inhibiting signalling mediated by binding of IL-11:IL-11Rα complex to gp130, and is also capable of inhibiting signalling mediated by binding of IL-11 to IL-11Rα:gp130 receptor.

In some embodiments, the agent may be capable of inhibiting a process mediated by IL-11.

In some embodiments, the agent may be capable of inhibiting gene/protein expression of IL-11 and/or IL-11Rα. Gene and/or protein expression can be measured as described herein or by methods in the art that will be well known to a skilled person.

In some embodiments, the agent may be capable of inhibiting gene/protein expression of IL-11 and/or IL-11Rα to less than 100′Y° , e.g. one of 99% or less, 95% or less, 90% or less, 85% or less, 80% or less, 75% or less, 70% or less, 65% or less, 60% or less, 55% or less, 50% or less, 45% or less, 40% or less, 35′Y° or less, 30% or less, 25% or less, 20% or less, 15% or less, 10% or less, 5% or less, or 1% or less of the level of expression in the absence of the agent (or in the presence of an appropriate control agent). In some embodiments, the agent is capable of inhibiting gene/protein expression of IL-11 and/or IL-11Rα to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of expression in the absence of the agent (or in the presence of an appropriate control agent).

Treatment/Prevention of Diseases and Conditions Characterised by Type IV Collagen Dysfunction

The present invention provides methods and articles (agents and compositions) for the treatment and/or prevention of diseases and conditions characterised by type IV collagen dysfunction, e.g. Alport syndrome.

Treatment is achieved by inhibition of IL-11-mediating signalling (i.e. antagonism of IL-11-mediated signalling). That is, the present invention provides for the treatment/prevention of diseases/conditions characterised by type IV collagen dysfunction (e.g. Alport syndrome) through inhibition of IL-11 mediated signalling, in e.g. a cell, tissue/organ/organ system/subject. In some embodiments, inhibition of IL-11-mediated signalling in accordance with the present disclosure comprises inhibition of IL-11-mediated signalling in the kidney.

Accordingly, the present invention provides an agent capable of inhibiting interleukin 11 (IL-11)-mediated signalling for use in a method of treating or preventing a disease/condition characterised by type IV collagen dysfunction (e.g. Alport syndrome).

Also provided is the use of an agent capable of inhibiting interleukin 11 (IL-11)-mediated signalling for use in the manufacture of a medicament for use in a method of treating or preventing a disease/condition characterised by type IV collagen dysfunction (e.g. Alport syndrome).

Further provided is a method of treating or preventing a disease/condition characterised by type IV collagen dysfunction (e.g. Alport syndrome), the method comprising administering to a subject in need of treatment a therapeutically effective amount of an agent capable of inhibiting interleukin 11 (IL-11)-mediated signalling.

The utility of the present invention extends to the treatment/prevention of any disease/condition characterised by type IV collagen dysfunction. The present invention also provides for the treatment/prevention of diseases/conditions that are caused or exacerbated by a disease/condition characterised by type IV collagen dysfunction (e.g. Alport syndrome). In some embodiments, the present invention provides for the treatment/prevention of diseases/conditions in a subject for which a disease/condition characterised by type IV collagen dysfunction (e.g. Alport syndrome) provides a poor prognosis.

In some embodiments, a disease/condition characterised by type IV collagen dysfunction (e.g. Alport syndrome) to be treated/prevented may be characterised by an increase in the expression of IL-11 and/or IL-11Rα (i.e. gene and/or protein expression) in an organ/tissue/subject affected by the disease/condition e.g. as compared to normal organ/tissue/subject (i.e. in the absence of the disease/condition).

A disease/condition characterised by type IV collagen dysfunction (e.g. Alport syndrome) according to the present invention may be associated with an upregulation of IL-11, e.g. an upregulation of IL-11 in cells or tissue in which the symptoms of the disease manifests or may occur, or upregulation of extracellular IL-11 or IL-11Rα.

The disease/condition characterised by type IV collagen dysfunction (e.g. Alport syndrome) may affect any tissue or organ or organ system. In some embodiments, the disease/condition may affect several tissues/organs/organ systems. In some embodiments, the disease/condition affects one or more of: the renal/urinary system, kidney, glomeruli, nervous system, auditory system, inner ear, cochlea, visual system, eye, lens or retina.

In accordance with the various aspects disclosed herein, in some embodiments the disease/condition is characterised by one or more of the following (relative to the healthy, non-diseased state): reduced renal function; reduced urine output; increased urinary albumin/creatinine ratio; increased serum creatinine level; increased serum urea level; increased blood urea nitrogen level; reduced kidney weight; increased renal fibrosis, e.g. tubulointerstitial fibrosis; increased renal gene/protein expression of one or more factors implicated in fibrosis (e.g. collagen, IL-11, fibronectin, αSMA, TGFβ); increased renal activation of ERK and/or STAT (i.e. increased levels of pSTAT and/or pERK in renal tissue); increased renal gene/protein expression of one or more factors implicated in the partial epithelial-to-mesenchymal transition of tubular epithelial cells (e.g. SNAIL); reduced renal gene/protein expression of one or more factors characteristic of the epithelial phenotype of tubular epithelial cells (e.g. E-cadherin); reduced number/proportion of tubular epithelial cells having an epithelial phenotype in renal tissue; reduced number/proportion of podocytes in renal tissue; reduced renal gene/protein expression of one or more factors expressed by podocytes (e.g. VVT1, podocin); increased apoptosis of podocytes; increased apoptosis of tubule epithelial cells; increased caspase activity in renal tissue; increased renal gene/protein expression of one or more markers of renal injury (e.g. KIM1, NGAL); and/or increased renal gene/protein expression of one or more markers of inflammation (e.g. IL-6, CCL2, CCL5, TNFα, IL-1β).

In accordance with the various aspects disclosed herein, in some embodiments the disease/condition is characterised by reduced/impaired function of the renal/urinary system relative to function in the absence of the disease/condition. In some embodiments the disease/condition is characterised by reduced/impaired function of the kidney, or reduced/impaired function of the glomeruli, relative to function in the absence of the disease/condition. In some embodiments, the disease/condition is characterised by glomerulonephritis, hematuria or proteinuria.

In accordance with the various aspects disclosed herein, in some embodiments the disease/condition is characterised by reduced/impaired function of the auditory system relative to function in the absence of the disease/condition. In some embodiments the disease/condition is characterised by reduced/impaired function of the inner ear or cochlea, relative to function in the absence of the disease/condition. In some embodiments, the disease/condition is characterised by deafness, e.g. sensorineural deafness, e.g. bilateral sensorineural deafness.

In accordance with the various aspects disclosed herein, in some embodiments the disease/condition is characterised by reduced/impaired function of the visual system relative to function in the absence of the disease/condition. In some embodiments the disease/condition is characterised by reduced/impaired function of the eye, lens or retina, relative to function in the absence of the disease/condition. In some embodiments, the disease/condition is characterised by an ocular abnormality, e.g. anterior lenticonus, posterior subcapsular cataract, posterior polymorphous dystrophy, or retinal flecks.

Treatment may be effective to reduce/delay/prevent the development or progression of a disease/condition characterised by type IV collagen dysfunction. Treatment may be effective to reduce/delay/prevent the worsening of one or more symptoms of a disease/condition characterised by type IV collagen dysfunction. Treatment may be effective to improve one or more symptoms of a disease/condition characterised by type IV collagen dysfunction. Treatment may be effective to reduce the severity of and/or reverse one or more symptoms of a disease/condition characterised by type IV collagen dysfunction. Treatment may be effective to reverse the effects of a disease/condition characterised by type IV collagen dysfunction.

Prevention may refer to prevention of development of a disease/condition characterised by type IV collagen dysfunction, and/or prevention of worsening of a disease/condition characterised by type IV collagen dysfunction, e.g. prevention of progression of a disease/condition characterised by type IV collagen dysfunction, e.g. to a later/chronic stage.

In some embodiments, the intervention may be aimed at slowing, stopping and/or reversing renal failure associated with a disease/condition characterised by type IV collagen dysfunction, e.g. Alport syndrome.

In accordance with various aspects of the present invention, a method of treating and/or preventing a disease/condition characterised by type IV collagen dysfunction according to the present invention may comprise increasing survival of a subject having a disease/condition characterised by type IV collagen dysfunction (e.g. Alport syndrome).

In accordance with various aspects of the present invention, methods are provided which are for, or which comprise (e.g. in the context of treatment/prevention of a disease/condition characterised by type IV collagen dysfunction, e.g. Alport syndrome), one or more of the following:
Increasing/maintaining kidney weight;
Increasing/maintaining renal function;
Increasing/maintaining urine output;
Reducing urinary albumin/creatinine ratio;
Reducing serum creatinine level;
Reducing serum urea level;
Reducing blood urea nitrogen level;
Increasing survival;
Reducing renal fibrosis, e.g. tubulointerstitial fibrosis;
Reducing renal gene/protein expression of one or more factors implicated in fibrosis (e.g. collagen, IL-11, fibronectin, αSMA, TGFβ);
Reducing renal activation of ERK and/or STAT (i.e. reducing levels of pSTAT and/or pERK in renal tissue);
Reducing renal gene/protein expression of one or more factors implicated in the partial epithelial-to-mesenchymal transition of tubular epithelial cells (e.g. SNAIL);
Increasing/maintaining renal gene/protein expression of one or more factors characteristic of the epithelial phenotype of tubular epithelial cells (e.g. E-cadherin);
Increasing/maintaining the number/proportion of tubular epithelial cells having an epithelial phenotype in renal tissue;
Increasing/maintaining the number/proportion of podocytes in renal tissue;
Increasing/maintaining renal gene/protein expression of one or more factors expressed by podocytes (e.g. WT1, podocin);
Reducing apoptosis of podocytes;
Reducing apoptosis of tubule epithelial cells;
Reducing caspase activity in renal tissue;
Reducing renal gene/protein expression of one or more markers of renal injury (e.g. KIM1, NGAL); and/or
Reducing renal gene/protein expression of one or more markers of inflammation (e.g. IL-6, CCL2, CCL5, TNFα, IL-162 ).

Also provided are agents according to the present disclosure for use in such methods, and the use of agents according to the present disclosure in manufacture of compositions (e.g. medicaments) for use in such methods. It will be appreciated that the methods typically comprise administering an agent capable of inhibiting IL-11-mediated signalling to a subject.

Similarly, one or more of the following may be observed in a subject following therapeutic or prophylactic intervention in accordance with the present disclosure (e.g. compared to the level/number/proportion etc. prior to intervention):
Increased/maintained kidney weight;
Increased/maintained renal function;
Increased/maintained urine output;
Reduced urinary albumin/creatinine ratio;
Reduced serum creatinine level;
Reduced serum urea level;
Reduced blood urea nitrogen level;
Reduced renal fibrosis, e.g. tubulointerstitial fibrosis;
Reduced renal gene/protein expression of one or more factors implicated in fibrosis (e.g. collagen, IL-11, fibronectin, αSMA, TGFβ);
Reduced renal activation of ERK and/or STAT (i.e. reduced levels of pSTAT and/or pERK in renal tissue);
Reduced renal gene/protein expression of one or more factors implicated in the partial epithelial-to-mesenchymal transition of tubular epithelial cells (e.g. SNAIL);
Increased/maintained renal gene/protein expression of one or more factors characteristic of the epithelial phenotype of tubular epithelial cells (e.g. E-cadherin);
Increased/maintained number/proportion of tubular epithelial cells having an epithelial phenotype in renal tissue;
Increased/maintained number/proportion of podocytes in renal tissue;
Increased/maintained renal gene/protein expression of one or more factors expressed by podocytes (e.g. WT1, podocin);
Reduced apoptosis of podocytes;
Reduced apoptosis of tubule epithelial cells;
Reduced caspase activity in renal tissue;
Reduced renal gene/protein expression of one or more markers of renal injury (e.g. KIM1, NGAL); and/or
Reduced renal gene/protein expression of one or more markers of inflammation (e.g. IL-6, CCL2, CCL5, TNFα, IL-1β).

In some embodiments, therapeutic/prophylactic intervention in accordance with the present disclosure may be described as being ‘associated with’ one or more of the effects described in the preceding paragraph. The skilled person is readily able to evaluate such properties using techniques that are routinely practiced in the art.

In some embodiments, treatment in accordance with the present disclosure may be effective to reverse one or more symptoms of a disease/condition characterised by type IV collagen dysfunction (e.g. Alport syndrome). Such treatment may be effective to reverse symptoms even in the case of established, advanced or severe disease/pathology.

In some embodiments, treatment in accordance with the present disclosure is effective to achieve one or more of the following in a subject having a disease/condition characterised by type IV collagen dysfunction (e.g. Alport syndrome): reverse disease/condition-associated reduction in kidney mass (i.e. increase kidney weight); reverse renal failure (i.e. increase and/or restore renal function); reverse disease/condition-associated reduction in urine output (i.e. increase urine output); reverse disease/condition-associated increase in urinary albumin/creatinine ratio (i.e. reduce urinary albumin/creatinine ratio); reverse disease/condition-associated increase in serum creatinine level (i.e. reduce serum creatinine level); reverse disease/condition-associated increase in serum urea level (i.e. reduce serum urea level); reverse disease/condition-associated increase in blood urea nitrogen level (i.e. reduce blood urea nitrogen level); reverse disease/condition-associated renal fibrosis (e.g. tubulointerstitial fibrosis) (i.e. reduce renal fibrosis (e.g. tubulointerstitial fibrosis)); reverse disease/condition-associated increase in renal gene/protein expression of one or more factors implicated in fibrosis (e.g. collagen, IL-11, fibronectin, αSMA, TGFβ) (i.e. reduce renal gene/protein expression of one or more factors implicated in fibrosis (e.g. collagen, IL-11, fibronectin, αSMA, TGFβ)); reverse disease/condition-associated increase in renal activation of ERK and/or STAT (e.g. disease/condition-associated increase levels of pSTAT and/or pERK in renal tissue) (i.e. reduce activation of ERK and/or STAT in renal tissue, and/or reduce levels of pSTAT and/or pERK in renal tissue); reverse disease/condition-associated increase in renal gene/protein expression of one or more factors implicated in the partial epithelial-to-mesenchymal transition of tubular epithelial cells (e.g. SNAIL) (i.e. reduce renal gene/protein expression of one or more factors implicated in the partial epithelial-to-mesenchymal transition of tubular epithelial cells (e.g. SNAIL)); reverse disease/condition-associated decrease in renal gene/protein expression of one or more factors characteristic of the epithelial phenotype of tubular epithelial cells (e.g. E-cadherin) (i.e. increase renal gene/protein expression of one or more factors characteristic of the epithelial phenotype of tubular epithelial cells (e.g. E-cadherin)); reverse disease/condition-associated reduction in the number/proportion of tubular epithelial cells having an epithelial phenotype in renal tissue (i.e. increase the number/proportion of tubular epithelial cells having an epithelial phenotype in renal tissue); reverse disease/condition-associated reduction in the number/proportion of podocytes in renal tissue (i.e. increase the number/proportion of podocytes in renal tissue); reverse disease/condition-associated decrease in renal gene/protein expression of one or more factors expressed by podocytes (e.g. WT1, podocin) (i.e. increase renal gene/protein expression of one or more factors expressed by podocytes (e.g. WT1, podocin)); reverse disease/condition-associated increase in apoptosis of podocytes (i.e. reduce apoptosis of podocytes); reverse disease/condition-associated increase in apoptosis of tubule epithelial cells (i.e. reduce apoptosis of tubule epithelial cells); reverse disease/condition-associated upregulation of caspase activity in renal tissue (i.e. reduce caspase activity in renal tissue); reverse disease/condition-associated increase in renal gene/protein expression of one or more markers of renal injury (e.g. KIM1, NGAL) (i.e. reduce renal gene/protein expression of one or more markers of renal injury (e.g. KIM1, NGAL)); and/or reverse disease/condition-associated increase in renal gene/protein expression of one or more markers of inflammation (e.g. IL-6, CCL2, CCL5, TNFα, IL-1β) (i.e. reduce renal gene/protein expression of one or more markers of inflammation (e.g. IL-6, CCL2, CCL5, TNFα, IL-1β)).

Administration

Administration of an agent capable of inhibiting IL-11-mediated signalling is preferably in a “therapeutically effective” or “prophylactically effective” amount, this being sufficient to show benefit to the subject.

The actual amount administered, and rate and time-course of administration, will depend on the nature and severity of the disease and the nature of the agent. Prescription of treatment, e.g. decisions on dosage etc., is within the responsibility of general practitioners and other medical doctors, and typically takes account of the disease/condition to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Multiple doses of the agent may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

In therapeutic applications, agents capable of inhibiting IL-11-mediated signalling are preferably formulated as a medicament or pharmaceutical together with one or more other pharmaceutically acceptable ingredients well known to those skilled in the art, including, but not limited to, pharmaceutically acceptable carriers, adjuvants, excipients, diluents, fillers, buffers, preservatives, anti-oxidants, lubricants, stabilisers, solubilisers, surfactants (e.g., wetting agents), masking agents, colouring agents, flavouring agents, and sweetening agents.

The term “pharmaceutically acceptable” as used herein pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, adjuvant, excipient, etc. must also be “acceptable” in the sense of being compatible with the other ingredients of the formulation.

Suitable carriers, adjuvants, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 2nd edition, 1994.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulations may be prepared for suitable administration in accordance with the disease/condition to be treated, e.g. topical, parenteral, systemic, intravenous, intra-arterial, intramuscular, intrathecal, intraocular, intra-conjunctival, subcutaneous, oral or transdermal routes of administration which may include injection. Injectable formulations may comprise the selected agent in a sterile or isotonic medium. The formulation and mode of administration may be selected according to the agent and disease to be treated.

Detection of IL-11 and Receptors for IL-11

Some aspects and embodiments of the present invention concern detection of expression of IL-11 or a receptor for IL-11 (e.g. IL-11Rα, gp130, or a complex containing IL-11Rα and/or gp130) in a sample obtained from a subject.

In some aspects and embodiments the present invention concerns the upregulation of expression (over-expression) of IL-11 or a receptor for IL-11 (as a protein or oligonucleotide encoding the respective IL-11 or receptor for IL-11) and detection of such upregulation as an indicator of suitability for treatment with an agent capable of inhibiting the action of IL-11 or with an agent capable of preventing or reducing the expression of IL-11 or a receptor for IL-11.

Upregulated expression comprises expression at a level that is greater than would normally be expected for a cell or tissue of a given type. Upregulation may be determined by measuring the level of expression of the relevant factor in a cell or tissue. Comparison may be made between the level of expression in a cell or tissue sample from a subject and a reference level of expression for the relevant factor, e.g. a value or range of values representing a normal level of expression of the relevant factor for the same or corresponding cell or tissue type. In some embodiments reference levels may be determined by detecting expression of IL-11 or a receptor for IL-11 in a control sample, e.g. in corresponding cells or tissue from a healthy subject or from healthy tissue of the same subject. In some embodiments reference levels may be obtained from a standard curve or data set.

Levels of expression may be quantitated for absolute comparison, or relative comparisons may be made.

In some embodiments upregulation of IL-11 or a receptor for IL-11 (e.g. IL-11Rα, gp130, or a complex containing IL-11Rα and/or gp130) may be considered to be present when the level of expression in the test sample is at least 1.1 times that of a reference level. More preferably, the level of expression may be selected from one of at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4 at least 2.5, at least 2.6, at least 2.7, at least 2.8, at least 2.9, at least 3.0, at least 3.5, at least 4.0, at least 5.0, at least 6.0, at least 7.0, at least 8.0, at least 9.0, or at least 10.0 times that of the reference level.

Expression levels may be determined by one of a number of known in vitro assay techniques, such as PCR based assays, in situ hybridisation assays, flow cytometry assays, immunological or immunohistochemical assays.

By way of example suitable techniques involve a method of detecting the level of IL-11 or a receptor for IL-11 in a sample by contacting the sample with an agent capable of binding IL-11 or a receptor for IL-11 and detecting the formation of a complex of the agent and IL-11 or receptor for IL-11. The agent may be any suitable binding molecule, e.g. an antibody, polypeptide, peptide, oligonucleotide, aptamer or small molecule, and may optionally be labelled to permit detection, e.g. visualisation, of the complexes formed. Suitable labels and means for their detection are well known to those in the art and include fluorescent labels (e.g. fluorescein, rhodamine, eosine and NDB, green fluorescent protein (GFP), chelates of rare earths such as europium (Eu), terbium (Tb) and samarium (Sm), tetramethyl rhodamine, Texas Red, 4-methyl umbelliferone, 7-amino-4-methyl coumarin, Cy3, Cy5), isotope markers, radioisotopes (e.g. 32P, 33P, 35S), chemiluminescence labels (e.g. acridinium ester, luminol, isoluminol), enzymes (e.g. peroxidase, alkaline phosphatase, glucose oxidase, beta-galactosidase, luciferase), antibodies, ligands and receptors. Detection techniques are well known to those of skill in the art and can be selected to correspond with the labelling agent. Suitable techniques include PCR amplification of oligonucleotide tags, mass spectrometry, detection of fluorescence or colour, e.g. upon enzymatic conversion of a substrate by a reporter protein, or detection of radioactivity.

Assays may be configured to quantify the amount of IL-11 or receptor for IL-11 in a sample. Quantified amounts of IL-11 or receptor for IL-11 from a test sample may be compared with reference values, and the comparison used to determine whether the test sample contains an amount of IL-11 or receptor for IL-11 that is higher or lower than that of the reference value to a selected degree of statistical significance.

Quantification of detected IL-11 or receptor for IL-11 may be used to determine up- or down-regulation or amplification of genes encoding IL-11 or a receptor for IL-11. In cases where the test sample contains fibrotic cells, such up-regulation, down-regulation or amplification may be compared to a reference value to determine whether any statistically significant difference is present.

A sample obtained from a subject may be of any kind. A biological sample may be taken from any tissue or bodily fluid, e.g. a blood sample, blood-derived sample, serum sample, lymph sample, semen sample, saliva sample, synovial fluid sample. A blood-derived sample may be a selected fraction of a patient's blood, e.g. a selected cell-containing fraction or a plasma or serum fraction. A sample may comprise a tissue sample or biopsy; or cells isolated from a subject. Samples may be collected by known techniques, such as biopsy or needle aspirate. Samples may be stored and/or processed for subsequent determination of IL-11 expression levels.

Samples may be used to determine the upregulation of IL-11 or receptor for IL-11 in the subject from which the sample was taken.

In some preferred embodiments a sample may be a tissue sample, e.g. biopsy, taken from a tissue/organ affected by a disease/condition characterised by type IV collagen dysfunction (e.g. Alport syndrome). A sample may contain cells.

A subject may be selected for therapy/prophylaxis in accordance with the present invention based on determination that the subject has an upregulated level of expression of IL-11 or of a receptor for IL-11 (e.g. IL-11Rα, gp130, or a complex containing IL-11Rα and/or gp130). Upregulated expression of IL-11 or of a receptor for IL-11 may serve as a marker of a disease/condition characterised by type IV collagen dysfunction suitable for treatment with an agent capable of inhibiting IL-11 mediated signalling.

Upregulation may be in a given tissue or in selected cells from a given tissue. A preferred tissue may be renal tissue. Upregulation of expression of IL-11 or of a receptor for IL-11 may also be determined in a circulating fluid, e.g. blood, or in a blood derived sample. Upregulation may be of extracellular IL-11 or IL-11Rα. In some embodiments expression may be locally or systemically upregulated.

Following selection, a subject may be administered with an agent capable of inhibiting IL-11 mediated signalling.

Diagnosis and Prognosis

Detection of upregulation of expression of IL-11 or a receptor for IL-11 (e.g. IL-11Rα, gp130, or a complex containing IL-11Rα and/or gp130) may also be used in a method of diagnosing a disease/condition characterised by type IV collagen dysfunction (e.g. Alport syndrome), identifying a subject at risk of developing a disease/condition characterised by type IV collagen dysfunction, and in methods of prognosing or predicting a subject's response to treatment with an agent capable of inhibiting IL-11 mediated signalling.

“Developing”, “development” and other forms of “develop” may refer to the onset of a disorder/disease, or the continuation or progression of a disorder/disease.

In some embodiments a subject may be suspected of having or suffering from a disease/condition characterised by type IV collagen dysfunction, e.g. based on the presence of other symptoms indicative of a disease/condition characterised by type IV collagen dysfunction in the subject's body or in selected cells/tissues of the subject's body, or be considered at risk of developing a disease/condition characterised by type IV collagen dysfunction, e.g. because of genetic predisposition or exposure to environmental conditions, known to be risk factors for a disease/condition characterised by type IV collagen dysfunction. Determination of upregulation of expression of IL-11 or a receptor for IL-11 may confirm a diagnosis or suspected diagnosis, or may confirm that the subject is at risk of developing a disease/condition characterised by type IV collagen dysfunction. The determination may also diagnose a disease/condition characterised by type IV collagen dysfunction or predisposition as one suitable for treatment with an agent capable of inhibiting IL-11-mediated signalling.

As such, a method of providing a prognosis for a subject having, or suspected of having a disease/condition characterised by type IV collagen dysfunction may be provided, the method comprising determining whether the expression of IL-11 or a receptor for IL-11 is upregulated in a sample obtained from the subject and, based on the determination, providing a prognosis for treatment of the subject with an agent capable of inhibiting IL-11-mediated signalling.

In some aspects, methods of diagnosis or methods of prognosing or predicting a subject's response to treatment with an agent capable of inhibiting IL-11-mediated signalling may not require determination of the expression of IL-11 or a receptor for IL-11, but may be based on determining genetic factors in the subject that are predictive of upregulation of expression or activity. Such genetic factors may include the determination of genetic mutations, single nucleotide polymorphisms (SNPs) or gene amplification in IL-11, IL-11Rα and/or gp130 which are correlated with and/or predictive of upregulation of expression or activity and/or IL-11 mediated signalling. The use of genetic factors to predict predisposition to a disease state or response to treatment is known in the art, e.g. see Peter Stärkel Gut 2008; 57:440-442; Wright et al., Mol. Cell. Biol. March 2010 vol. 30 no. 6 1411-1420.

Genetic factors may be assayed by methods known to those of ordinary skill in the art, including PCR based assays, e.g. quantitative PCR, competitive PCR. By determining the presence of genetic factors, e.g. in a sample obtained from a subject, a diagnosis may be confirmed, and/or a subject may be classified as being at risk of developing a disease/condition described herein, and/or a subject may be identified as being suitable for treatment with an agent capable of inhibiting IL-11 mediated signalling.

Some methods may comprise determination of the presence of one or more SNPs linked to secretion of IL-11 or susceptibility to development of a disease/condition characterised by type IV collagen dysfunction. SNPs are usually bi-allelic and therefore can be readily determined using one of a number of conventional assays known to those of skill in the art (e.g. see Anthony J. Brookes. The essence of SNPs. Gene Volume 234, Issue 2, 8 July 1999, 177-186; Fan et al., Highly Parallel SNP Genotyping. Cold Spring Harb Symp Quant Biol 2003. 68: 69-78; Matsuzaki et al., Parallel Genotyping of Over 10,000 SNPs using a one-primer assay on a high-density oligonucleotide array. Genome Res. 2004. 14: 414-425).

The methods may comprise determining which SNP allele is present in a sample obtained from a subject. In some embodiments determining the presence of the minor allele may be associated with increased IL-11 secretion or susceptibility to development of a disease/condition characterised by type IV collagen dysfunction.

Accordingly, in one aspect of the present invention a method for screening a subject is provided, the method comprising:
obtaining a nucleic acid sample from the subject;
determining which allele is present in the sample at the polymorphic nucleotide position of one or more of the SNPs listed in FIG. 33, FIG. 34, or FIG. 35 of WO 2017/103108 A1 (incorporated by reference herein), or a SNP in linkage disequilibrium with one of the listed SNPs with an r²≥0.8.

The determining step may comprise determining whether the minor allele is present in the sample at the selected polymorphic nucleotide position. It may comprise determining whether 0, 1 or 2 minor alleles are present.

The screening method may be, or form part of, a method for determining susceptibility of the subject to development of a disease/condition characterised by type IV collagen dysfunction, or a method of diagnosis or prognosis as described herein.

The method may further comprise the step of identifying the subject as having susceptibility to, or an increased risk of, developing a disease/condition characterised by type IV collagen dysfunction, e.g. if the subject is determined to have a minor allele at the polymorphic nucleotide position. The method may further comprise the step of selecting the subject for treatment with an agent capable of inhibiting IL-11 mediated signalling and/or administering an agent capable of inhibiting IL-11 mediated signalling to the subject in order to provide a treatment for a disease/condition characterised by type IV collagen dysfunction in the subject or to prevent development or progression of a disease/condition characterised by type IV collagen dysfunction in the subject.

In some embodiments, a method of diagnosing a disease/condition characterised by type IV collagen dysfunction, identifying a subject at risk of developing a disease/condition characterised by type IV collagen dysfunction, and methods of prognosing or predicting a subject's response to treatment with an agent capable of inhibiting IL-11 mediated signalling employs an indicator that is not detection of upregulation of expression of IL-11 or a receptor for IL-11, or genetic factors.

In some embodiments, a method of diagnosing a disease/condition characterised by type IV collagen dysfunction, identifying a subject at risk of developing a disease/condition characterised by type IV collagen dysfunction, and methods of prognosing or predicting a subject's response to treatment with an agent capable of inhibiting IL-11 mediated signalling is based on detecting, measuring and/or identifying one or more indicators of type IV collagen function.

Methods of diagnosis or prognosis may be performed in vitro on a sample obtained from a subject, or following processing of a sample obtained from a subject. Once the sample is collected, the patient is not required to be present for the in vitro method of diagnosis or prognosis to be performed and therefore the method may be one which is not practised on the human or animal body. The sample obtained from a subject may be of any kind, as described herein above.

Other diagnostic or prognostic tests may be used in conjunction with those described here to enhance the accuracy of the diagnosis or prognosis or to confirm a result obtained by using the tests described here.

Subjects

Subjects may be animal or human. Subjects are preferably mammalian, more preferably human. The subject may be a non-human mammal, but is more preferably human. The subject may be male or female. The subject may be a patient.

The patient may have a disease/condition characterised by type IV collagen dysfunction (e.g. Alport syndrome) as described herein. A subject may have been diagnosed with a disease/condition characterised by type IV collagen dysfunction requiring treatment, may be suspected of having such a disease/condition characterised by type IV collagen dysfunction, or may be at risk from developing a disease/condition characterised by type IV collagen dysfunction.

In embodiments according to the present invention the subject is preferably a human subject. In embodiments according to the present invention, a subject may be selected for treatment according to the methods based on characterisation for certain markers of a disease/condition characterised by type IV collagen dysfunction.

Sequence Identity

Pairwise and multiple sequence alignment for the purposes of determining percent identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such software, the default parameters, e.g. for gap penalty and extension penalty, are preferably used.

Sequences

| SEQ ID | | |
|---|---|---|
| NO: | DESCRIPTION | SEQUENCE |
| 1 | Human IL-11 (UniProt | MNCVCRLVLVVLSLWPDTAVAPGPPPGPPRVSPDPRAELDSTVLLTRSLLADTRQLA |
| | P20809) | AQLRDKFPADGDHNLDSLPTLAMSAGALGALQLPGVLTRLRADLLSYLRHVQWLRR |
| | | AGGSSLKTLEPELGTLQARLDRLLRRLQLLMSRLALPQPPPDPPAPPLAPPSSAWG |
| | | GIRAAHAILGGLHLTLDWAVRGLLLLKTRL |
| 2 | Human gp130 | MLTLQTWLVQALFIFLTTESTGELLDPCGYISPESPVVQLHSNFTAVCVLKEKCMDYF |
| | (UniProt P40189-1) | HVNANYIVWKTNHFTIPKEQYTIINRTASSVTFTDIASLNIQLTCNILTFGQLEQNVYG |
| | | ITIISGLPPEKPKNLSCIVNEGKKMRCEWDGGRETHLETNFTLKSEWATHKFADCKAKR |
| | | DTPTSCTVDYSTVYFVNIEVWVEAENALGKVTSDHINFDPVYKVKPNPPHNLSVINSE |
| | | ELSSILKLTWTNPSIKSVIILKYNIQYRTKDASTWSQIPPEDTASTRSSFTVQDLKPF |
| | | TEYVFRIRCMKEDGKGYWSDWSEEASGITYEDRPSKAPSFWYKIDPSHTQGYRTVQLV |
| | | WKTLPPFEANGKILDYEVTLTRWKSHLQNYTVNATKLTVNLTNDRYLATLTVRNLVG |
| | | KSDAAVLTIPACDFQATHPVMDLKAFPKDNMLWVEWTTPRESVKKYILEWCVLSDK |
| | | APCITDWQQEDGTVHRTYLRGNLAESKCYLITVTPVYADGPGSPESIKAYLKQAPPS |
| | | KGPTVRTKKVGKNEAVLEWDQLPVDVQNGFIRNYTIFYRTIIGNETAVNVDSSHTEYT |
| | | LSSLTSDTLYMVRMAAYTDEGGKDGPEFTFTTPKFAQGEIEAIVVPVCLAFLLTTLLG |
| | | VLFCFNKRDLIKKHIWPNVPDPSKSHIAQWSPHTPPRHNFNSKDQMYSDGNFTDVS |
| | | WEIEANDKKPFPEDLKSLDLFKKEKINTEGHSSGIGGSSCMSSSRPSISSSDENESS |
| | | QNTSSTVQYSTVVHSGYRHQVPSVQVFSRSESTQPLLDSEERPEDLQLVDHVDGG |
| | | DGILPRQQYFKQNCSQHESSPDISHFERSKQVSSVNEEDFVRLKQQISDHISQSCGS |
| | | GQMKMFQEVSAADAFGPGTEGQVERFETVGMEAATDEGMPKSYLPQTVRQGGYM |
| | | PQ |
| 3 | Human IL11RA | MSSSCSGLSRVLVAVATALVSASSPCPQAWGPPGVQYGQPGRSVKLCCPGVTAGD |
| | (UniProt Q14626) | PVSWFRDGEPKLLQGPDSGLGHELVLAQADSTDEGTYICQTLDGALGGTVTLQLGY |
| | | PPARPVVSCQAADYENFSCTWSPSQISGLPTRYLTSYRKKTVLGADSQRRSPSTGP |
| | | WPCPQDPLGAARCVVHGAEFWSQYRINVTEVNPLGASTRLLDVSLQSILRPDPPQG |
| | | LRVESVPGYPRRLRASWTYPASWPCQPHFLLKFRLQYRPAQHPAWSTVEPAGLEE |
| | | VITDAVAGLPHAVRVSARDFLDAGTWSTWSPEAWGTPSTGTIPKEIPAWGQLHTQP |
| | | EVEPQVDSPAPPRPSLQPHPRLLDHRDSVEQVAVLASLGILSFLGLVAGALALGLWL |
| | | RLRRGGKDGSPKPGFLASVIPVDRRPGAPNL |
| 4 | siRNA target IL-11 | CCTTCCAAAGCCAGATCTT |
| 5 | siRNA target IL-11 | GCCTGGGCAGGAACATATA |
| 6 | siRNA target IL-11 | CCTGGGCAGGAACATATAT |
| 7 | siRNA target IL-11 | GGTTCATTATGGCTGTGTT |
| 8 | siRNA target IL-11 Rα | GGACCATACCAAAGGAGAT |
| 9 | siRNA target IL-11 Rα | GCGTCTTTGGGAATCCTTT |
| 10 | siRNA target IL-11 Rα | GCAGGACAGTAGATCCCT |
| 11 | siRNA target IL-11 Rα | GCTCAAGGAACGTGTGTAA |
| 12 | siRNA to IL-11 | CCUUCCAAAGCCAGAUCUUdTdT-AAGAUCUGGCUUUGGAAGGdTdT |
| | (NM_000641.3) | |
| 13 | siRNA to IL-11 | GCCUGGGCAGGAACAUAUAdTdT-UAUAUGUUCCUGCCCAGGCdTdT |
| | (NM_000641.3) | |
| 14 | siRNA to IL-11 | CCUGGGCAGGAACAUAUAUdTdT-AUAUAUGUUCCUGCCCAGGdTdT |
| | (NM_000641.3) | |
| 15 | siRNA to IL-11 | GGUUCAUUAUGGCUGUGUUdTdT-AACACAGCCAUAAUGAACCdTdT |
| | (NM_000641.3) | |
| 16 | siRNA to IL-11Rα | GGACCAUACCAAAGGAGAUdTdT-AUCUCCUUUGGUAUGGUCCdTdT |
| | (U32324.1) | |
| 17 | siRNA to IL-11Rα | GCGUCUUUGGGAAUCCUUUdTdT-AAAGGAUUCCCAAAGACGCdTdT |
| | (U32324.1) | |
| 18 | siRNA to IL-11Rα | GCAGGACAGUAGAUCCCUAdTdT-UAGGGAUCUACUGUCCUGCdTdT |
| | (U32324.1) | |
| 19 | siRNA to IL-11Rα | GCUCAAGGAACGUGUGUAAdTdT-UUACACACGUUCCUUGAGCdTdT |
| | (U32324.1) | |
| 20 | 20 amino acid linker | GPAGQSGGGGGSGGGSGGGSV |
| 21 | Hyper IL-11 (IL- | MSSSCSGLSRVLVAVATALVSASSPCPQAWGPPGVQYGQPGRSVKLCCPGVTAGD |
| | 11 RA: IL-11 fusion) | PVSWFRDGEPKLLQGPDSGLGHELVLAQADSTDEGTYICQTLDGALGGTVTLQLGY |
| | | PPARPVVSCQAADYENFSCTWSPSQISGLPTRYLTSYRKKTVLGADSQRRSPSTGP |
| | | WPCPQDPLGAARCVVHGAEFWSQYRINVTEVNPLGASTRLLDVSLQSILRPDPPQG |
| | | LRVESVPGYPRRLRASWTYPASWPCQPHFLLKFRLQYRPAQHPAWSTVEPAGLEE |
| | | VITDAVAGLPHAVRVSARDFLDAGTWSTWSPEAWGTPSTGPAGQSGGGGGSGGG |
| | | SGGGSVPGPPPGPPRVSPDPRAELDSTVLLTRSLLADTRQLAAQLRDKFPADGDHN |
| | | LDSLPTLAMSAGALGALQLPGVLTRLRADLLSYLRHVQWLRRAGGSSLKTLEPELGT |
| | | LQARLDRLLRRLQLLMSRLALPQPPPDPPAPPLAPPSSAWGGIRAAHAILGGLHLTLD |
| | | WAVRGLLLLKTRL |
| 22 | Enx203 VH | EVQLQQSGPELVKPGASVKIPCKASGYTFTDYNMDWVKQSHGKSLEWIGDINPHNG |
| | | GPIYNQKFTGKATLTVDKSSSTAYMELRSLTSEDTAVYYCARGELGHWYFDVWGTG |
| | | TTVTVSS |
| 23 | Enx203 VL | DIVLTQSPASLAVSLGQRATISCRASKSVSTSGYSYIHWYQQKPGQPPKLLIYLASNL |
| | | DSGVPARFSGSGSGTDFTLNIHPVEEEDAATYYCQHSRDLPPTFGGGTKLEIK |
| 24 | Enx209 VH | QVQLQQPGAELVRPGSSVKLSCKASGYTFTNYWMHWLKQRPVQGLEWIGNIGPSD |
| | | SKTHYNQKFKDKATLTVDKSSSTAYMQLNSLTSEDSAVYYCARGDYVLFTYWGQGT |
| | | LVTVSA |
| 25 | Enx209 VL | DIVLTQSPATLSLSPGERATLSCRASQSISNNLHWYQQKSHEAPRLLIKYASQSISGIP |
| | | ARFSGSGSGTDFTLSFSSLETEDFAVYFCQQSYSWPLTFGQGTKLEIK |
| 26 | Enx108A VH | QVQLVQSGGGVVQPGRSLRLSCAASGFTFSSYGMHWVRQAPGKGLEWVAVISYD |
| | | GSNKYYADSVKGRFTISRDNSKNTLYLQMNSLRAEDTAVYYCAKIGATDPLDYWGQ |
| | | GTLVTVSS |
| 27 | Enx108A VL | QSALTQPRSVSGSPGQSVTLSCTGTSSDVGGYNYVSWYQHYPGKAPKLMIFDVNE |
| | | RSSGVPDRFSGSKSGNTASLTISGLQAEDEADYYCASYAGRYTWMFGGGTKVTVL |
| | | G |
| 28 | Enx108A hIgG4 | QVQLVQSGGGVVQPGRSLRLSCAASGFTFSSYGMHWVRQAPGKGLEWVAVISYD |
| | (L248E, S241P) HC | GSNKYYADSVKGRFTISRDNSKNTLYLQMNSLRAEDTAVYYCAKIGATDPLDYWGQ |
| | | GTLVTVSSASTKGPSVFPLAPCSRSTSESTAALGCLVKDYFPEPVTVSWNSGALTSG |
| | | VHTFPAVLQSSGLYSLSSVVTVPSSSLGTKTYTCNVDHKPSNTKVDKRVESKYGPPC |
| | | PPCPAPEFEGGPSVFLFPPKPKDTLMISRTPEVTCVVVDVSQEDPEVQFNWYVDGV |
| | | EVHNAKTKPREEQFNSTYRVVSVLTVLHQDWLNGKEYKCKVSNKGLPSSIEKTISKA |
| | | KGQPREPQVYTLPPSQEEMTKNQVSLTCLVKGFYPSDIAVEWESNGQPENNYKTTP |
| | | PVLDSDGSFFLYSRLTVDKSRWQEGNVFSCSVMHEALHNHYTQKSLSLSLGK |
| 29 | Enx108A lambda LC | QSALTQPRSVSGSPGQSVTLSCTGTSSDVGGYNYVSWYQHYPGKAPKLMIFDVNE |
| | | RSSGVPDRFSGSKSGNTASLTISGLQAEDEADYYCASYAGRYTWMFGGGTKVTVL |
| | | GQPKAAPSVTLFPPSSEELQANKATLVCLISDFYPGAVTVAWKADSSPVKAGVETTT |
| | | PSKQSNNKYAASSYLSLTPEQWKSHRSYSCQVTHEGSTVEKTVAPTECS |
| 30 | hEnx203 VH | EVQLVQSGAEVKKPGASVKISCKASGYTFTDYNMDWVKQAPGQRLEWIGDINPHNG |
| | | GPIYNQKFTGRATLTVDKSASTAYMELSSLRSEDTAVYYCARGELGHWYFDVWGQ |
| | | GTTVTVSS |
| 31 | hEnx203 VL | DIVLTQSPASLALSPGERATLSCRASKSVSTSGYSYIHWYQQKPGQAPRLLIYLASNL |
| | | DSGVPARFSGSGSGTDFTLTISSLEEEDFATYYCQHSRDLPPTFGQGTKLEIK |
| 32 | hEnx209 VH | QVQLVQSGAEVKKPGASVKVSCKASGYTFTNYWMHWLRQRPGQGLEWIGNIGPSD |
| | | SKTHYNQKFKDRVTMTVDKSTSTAYMELSSLRSEDTAVYYCARGDYVLFTYWGQG |
| | | TLVTVSS |
| 33 | hEnx209 VL | DIVLTQSPATLSLSPGERATLSCRASQSISNNLHWYQQKPGQAPRLLIKYASQSISGI |
| | | PARFSGSGSGTDFTLTISSLEPEDFAVYYCQQSYSWPLTFGQGTKLEIK |
| 34 | Enx108A VH CDR1 | SYGMH |
| 35 | Enx108A VH CDR2 | VISYDGSNKYYADSVKG |
| 36 | Enx108A VH CDR3 | IGATDPLDY |
| 37 | Enx108A VL CDR1 | TGTSSDVGGYNYVS |
| 38 | Enx108A VL CDR2 | DVNERSS |
| 39 | Enx108A VL CDR3 | ASYAGRYTWM |
| 40 | Enx203, hEnx203 VH | DYNMD |
| | CDR1 | |
| 41 | Enx203, hEnx203 VH | DINPHNGGPIYNQKFTG |
| | CDR2 | |
| 42 | Enx203, hEnx203 VH | GELGHWYFDV |
| | CDR3 | |
| 43 | Enx203, hEnx203 VL | RASKSVSTSGYSYIH |
| | CDR1 | |
| 44 | Enx203, hEnx203 VL | LASNLDS |
| | CDR2 | |
| 45 | Enx203, hEnx203 VL | QHSRDLPPT |
| | CDR3 | |
| 46 | Enx209, hEnx209 VH | NYWMH |
| | CDR1 | |
| 47 | Enx209, hEnx209 VH | NIGPSDSKTHYNQKFKD |
| | CDR2 | |
| 48 | Enx209, hEnx209 VH | GDYVLFTY |
| | CDR3 | |
| 49 | Enx209, hEnx209 VL | RASQSISNNLH |
| | CDR1 | |
| 50 | Enx209, hEnx209 VL | YASQSIS |
| | CDR2 | |
| 51 | Enx209, hEnx209 VL | QQSYSWPLT |
| | CDR3 | |
| 52 | Human IGHG1 | ASTKGPSVFPLAPSSKSTSGGTAALGCLVKDYFPEPVTVSWNSGALTSGVHTFPAVL |
| | constant (K214R, | QSSGLYSLSSVVTVPSSSLGTQTYICNVNHKPSNTKVDKRVEPKSCDKTHTCPPCPA |
| | D356E, L358M) | PELLGGPSVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGK |
| 53 | Human IGHG4 | ASTKGPSVFPLAPCSRSTSESTAALGCLVKDYFPEPVTVSWNSGALTSGVHTFPAVL |
| | constant (L248E, | QSSGLYSLSSVVTVPSSSLGTKTYTCNVDHKPSNTKVDKRVESKYGPPCPPCPAPE |
| | S241P) | FEGGPSVFLFPPKPKDTLMISRTPEVTCVVVDVSQEDPEVQFNWYVDGVEVHNAKT |
| | | KPREEQFNSTYRVVSVLTVLHQDWLNGKEYKCKVSNKGLPSSIEKTISKAKGQPREP |
| | | QVYTLPPSQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK |
| 54 | Human IGKC | RTVAAPSVFIFPPSDEQLKSGTASVVCLLNNFYPREAKVQWKVDNALQSGNSQESV |
| | constant | TEQDSKDSTYSLSSTLTLSKADYEKHKVYACEVTHQGLSSPVTKSFNRGEC |
| 55 | Human IGLC2 | GQPKAAPSVTLFPPSSEELQANKATLVCLISDFYPGAVTVAWKADSSPVKAGVETTT |
| | constant | PSKQSNNKYAASSYLSLTPEQWKSHRSYSCQVTHEGSTVEKTVAPTECS |
| 56 | hEnx203 hIgG1 HC | EVQLVQSGAEVKKPGASVKISCKASGYTFTDYNMDWVKQAPGQRLEWIGDINPHNG |
| | | GPIYNQKFTGRATLTVDKSASTAYMELSSLRSEDTAVYYCARGELGHWYFDVWGQ |
| | | GTTVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVSNKALPAPIEKTI |
| | | SKAKGQPREPQVYTLPPSREEMTKNQVSLTCLVKGFYPSDIAVEWESNGQPENNYK |
| | | TTPPVLDSDGSFFLYSKLTVDKSRWQQGNVFSCSVMHEALHNHYTQKSLSLSPGK |
| 57 | hEnx203 kappa LC | DIVLTQSPASLALSPGERATLSCRASKSVSTSGYSYIHWYQQKPGQAPRLLIYLASNL |
| | | DSGVPARFSGSGSGTDFTLTISSLEEEDFATYYCQHSRDLPPTFGQGTKLEIKRTVA |
| | | APSVFIFPPSDEQLKSGTASVVCLLNNFYPREAKVQWKVDNALQSGNSQESVTEQD |
| | | SKDSTYSLSSTLTLSKADYEKHKVYACEVTHQGLSSPVTKSFNRGEC |
| 58 | hEnx209 hIgG4 | QVQLVQSGAEVKKPGASVKVSCKASGYTFTNYWMHWLRQRPGQGLEWIGNIGPSD |
| | (L248E, S241P) HC | SKTHYNQKFKDRVTMTVDKSTSTAYMELSSLRSEDTAVYYCARGDYVLFTYWGQG |
| | | TLVTVSSASTKGPSVFPLAPCSRSTSESTAALGCLVKDYFPEPVTVSWNSGALTSGV |
| | | HTFPAVLQSSGLYSLSSVVTVPSSSLGTKTYTCNVDHKPSNTKVDKRVESKYGPPCP |
| | | PCPAPEFEGGPSVFLFPPKPKDTLMISRTPEVTCVVVDVSQEDPEVQFNWYVDGVE |
| | | VHNAKTKPREEQFNSTYRVVSVLTVLHQDWLNGKEYKCKVSNKGLPSSIEKTISKAK |
| | | GQPREPQVYTLPPSQEEMTKNQVSLTCLVKGFYPSDIAVEWESNGQPENNYKTTPP |
| | | VLDSDGSFFLYSRLTVDKSRWQEGNVFSCSVMHEALHNHYTQKSLSLSLGK |
| 59 | hEnx209 kappa LC | DIVLTQSPATLSLSPGERATLSCRASQSISNNLHWYQQKPGQAPRLLIKYASQSISGI |
| | | PARFSGSGSGTDFTLTISSLEPEDFAVYYCQQSYSWPLTFGQGTKLEIKRTVAAPSV |
| | | FIFPPSDEQLKSGTASVVCLLNNFYPREAKVQWKVDNALQSGNSQESVTEQDSKDS |
| | | TYSLSSTLTLSKADYEKHKVYACEVTHQGLSSPVTKSFNRGEC |

The invention includes the combination of the aspects and preferred features described except where such a combination is clearly impermissible or expressly avoided.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

Methods disclosed herein may be performed, or products may be present, in vitro, ex vivo, or in vivo. The term “in vitro” is intended to encompass experiments with materials, biological substances, cells and/or tissues in laboratory conditions or in culture whereas the term “in vivo” is intended to encompass experiments and procedures with intact multi-cellular organisms. In some embodiments, methods performed in vivo may be performed on non-human animals. “Ex vivo” refers to something present or taking place outside an organism, e.g. outside the human or animal body, which may be on tissue (e.g. whole organs) or cells taken from the organism.

Where a nucleic acid sequence is disclosed herein, the reverse complement thereof is also expressly contemplated.

For standard molecular biology techniques, see Sambrook, J., Russel, D.W. Molecular Cloning, A Laboratory Manual. 3 ed. 2001, Cold Spring Harbor, New York: Cold Spring Harbor Laboratory Press

Aspects and embodiments of the present invention will now be discussed with reference to the accompanying figures. Further aspects and embodiments will be apparent to those skilled in the art. All documents mentioned in this text are incorporated herein by reference in their entirety. While the invention has been described in conjunction with the exemplary embodiments described below, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

### BRIEF DESCRIPTION OF THE FIGURES

Embodiments and experiments illustrating the principles of the invention will now be discussed with reference to the accompanying figures.

FIGS. 1A and 1B. Graph and table showing the results of treatment with anti-IL-11 antibody antagonist of IL-11 mediated signalling on survival of subjects in a mouse model of Alport syndrome. (1A) Graph showing survival of subjects over time. (1B) Table summarising the results of statistical analysis of the survival curves of FIG. 1A.

FIGS. 2A to 2E. Graphs and images showing that IL11 is upregulated in kidneys of Col4a3^{-/-} mice and IL11RA is expressed in podocytes and renal tubular epithelial cells. (2A-2C) Renal (2A) II11 RNA and (2B-2C) IL11 protein expression in wildtype and Col4a3^{-/-} mice. (2D) Immunohistochemistry staining of IL11RA with anti-IL11RA (X209) or IgG (11E10) as control on the kidneys of wild-type and II11ra1^{-/-} mice (scale bars, 20 μm). (2E) Comparison of II11ra1 and gp130 expression in mouse kidney cells based on single cell transcriptomic analysis by Park et. al.¹⁶. (2A) Data are shown as box-and-whisker with median (middle line), 25^{th}-75^{th} percentiles (box) and min-max values (whiskers), (2C) data are shown as mean±SD; 2-tailed Student's t-test. FC: fold change.

FIGS. 3A to 3G. Schematic, graphs and images showing that in Col4a3^{-/-} mice, a neutralizing IL11 antibody reduces renal ERK and STAT activation, fibrosis and a signature of epithelial-to-mesenchymal transition. (3A) Schematic showing therapeutic dosing of Col4a3^{-/-} mice for experimental data shown in B-I. 6-week-old Col4a3^{-/-} mice were administered IgG/X203 (20 mg/kg, 2×/week) for 2.5 weeks wild-type littermates were used as controls. (3B) Body weight (shown as a percentage (%) of initial body weight). (3C) Kidney weight. (3D) Total renal collagen content. (3E) Representative and (3F) quantification (from 100× field images) of Masson Trichrome's staining. (3G) Relative renal mRNA expression of pro-fibrotic markers (Col1a1, Col3a1, II11, Col1a2, Fn, Acta2, and Tgff3). (3H) Western blots and (3I) densitometry analysis of p-ERK, ERK, p-STAT3, STAT3, αSMA, Fibronectin, E-cadherin, SNAIL, and GAPDH. (3B, 3F, 3I) Data are shown as mean±SD, (3C, 3D, 3G) data are shown as box-and-whisker with median (middle line), 25^{th}-75^{th} percentiles (box) and min-max values (whiskers). (3B) 2-way ANOVA with Tukey's correction, (3C, 3D, 3F, 3G, 3I) one-way ANOVA with Tukey's correction. FC: fold change.

FIGS. 4A to 4E. Images and graphs showing that inhibition of IL11 signaling with a neutralizing IL11 antibody preserves podocytes and reduces renal inflammation and tubule damage in Col4a3^{-/-} mice. 4A-4E show Data for experiments shown in schematic FIG. 3A. (4A) Representative and (4B) quantification (from 200× field images) of Wilms' Tumor 1 (VVT1) staining. (4C) Western blots and (4D) densitometry analysis of TGFβ, Cleaved Caspase 3, Caspase 3, Podocin, WT1, and GAPDH. (4E) Relative renal mRNA expression of kidney injury markers (Kim1 and Nga1), podocyte marker (Podocin), and pro-inflammation markers (II6, Cc12, Cc15, Tnfα, and II1β). (4B, 4E) Data are shown as box-and-whisker with median (middle line), 25^{th}-75^{th} percentiles (box) and min-max values (whiskers), (4D) data are shown as mean±SD; one-way ANOVA with Tukey's correction. FC: fold change

FIGS. 5A to 5E. Graphs and schematic showing that therapeutic targeting of IL11 in Col4a3^{-/-} mice improves renal function and prolongs median lifespan. (5A-5C) Data for experiments shown in schematic FIG. 3A; data are shown as box-andwhisker with median (middle line), 25^{th}-75^{th} percentiles (box) and min-max values (whiskers); one-way ANOVA with Tukey's correction. (5A) Blood urea nitrogen (BUN). (5B) Serum Creatinine. (5C) Urinary Albumin:Creatinine ratios. (5D) Schematic showing therapeutic dosing of Col4a3^{-/-} mice in lifespan study. Col4a3^{-/-} mice were administered IgG/X203 (20 mg/kg, 2×/week) starting from 6 weeks of age until death ensued. (5E) Survival curves of mice treated with either IgG or X203 for experiments shown in 5D; Gehan-Breslow-Wilcoxon test.

### EXAMPLES

In the following Examples, the inventors demonstrate that inhibition of IL-11-mediated signalling reduces increases survival of subjects in a model of Alport syndrome.

The inventors show that the fibro-inflammatory cytokine interleukin 11 (IL11) is upregulated in the kidneys of Col4a3^{-/-} mice, and that the receptor for IL11 (IL11RA1) is expressed on podocytes and tubule cells. Administering Col4a3^{-/-} mice with neutralizing IL11 antibody (X203) at 6-weeks of age (a time when ACE inhibition is no longer effective in slowing progression of kidney disease in this model) is shown to reduce kidney fibrosis, inflammation and tubule damage, improve kidney function and extend lifespan by 41.6%. Given the excellent translatability of Col4a3^{-/-} mouse as a model of Alport syndrome, the data indicate that IL11-targeted therapies are useful for the treatment of Alport syndrome in humans.

### Example 1: Analysis of the Effect of Antagonism of IL-11-Mediated Signalling In Vivo in a Mouse Model of Alport Syndrome

The inventors investigated the effect of administration of an antagonist of IL-11-mediated signalling on survival of subjects in a mouse model of Alport syndrome.

129-Col4a3^{tm1Dec}/J mice were obtained from The Jackson Laboratory (Stock No:002908; COL4A3 KO). The mice comprise the Co/4a3^{tm1Dec} mutation described e.g. in Cosgrove et al., Genes Dev. (1996) 10:2981-2992, which is hereby incorporated by reference in its entirety. 129-Col4a3^{tm1Dec}/J mice comprise a targeted mutation to Col4a3, resulting in progressive glomerulonephritis with microhematuria and proteinuria, and mice homozygous for the mutation typically die at about 8.5 weeks.

From 6 weeks of age, mice homozygous for the Co/4a3^{tm1Dec} mutation were administered biweekly by intraperitoneal injection of 20 mg/kg of Enx203, or an isotype-matched control antibody, until mortality.

Enx203 is a mouse anti-mouse IL-11 IgG, and is described e.g. in Ng et al., Sci Transl Med. (2019) 11(511) pii: eaaw1237 (also published as Ng, et al., “IL-11 is a therapeutic target in idiopathic pulmonary fibrosis.” bioRxiv 336537; doi: https://doi.orq/10.1101/336537). Enx203 is also referred to as “X203”. Enx203 comprises the VH region according to SEQ ID NO:92 of WO 2019/238882 A1 (SEQ ID NO:22 of the present disclosure), and the VL region according to SEQ ID NO:94 of WO 2019/238882 Al (SEQ ID NO:23 of the present disclosure).

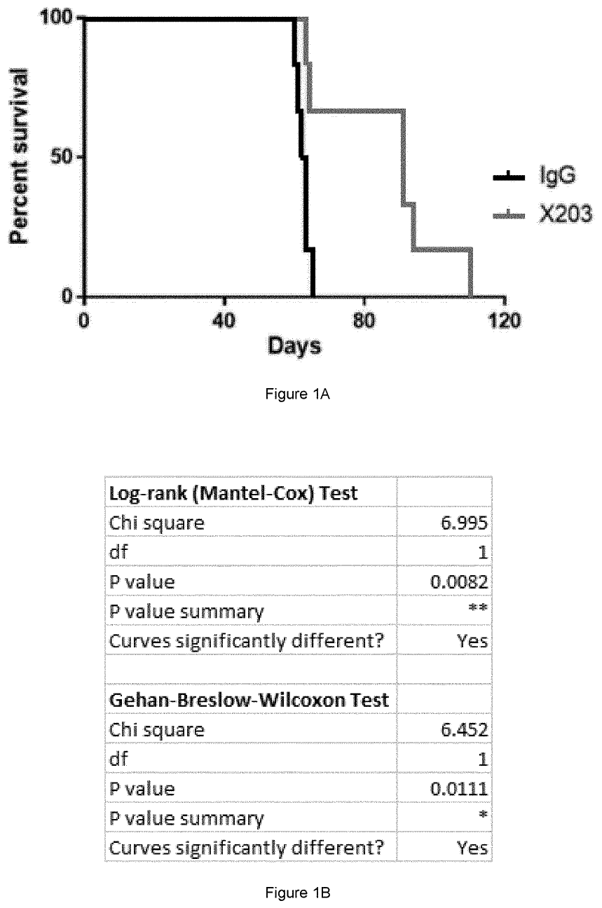

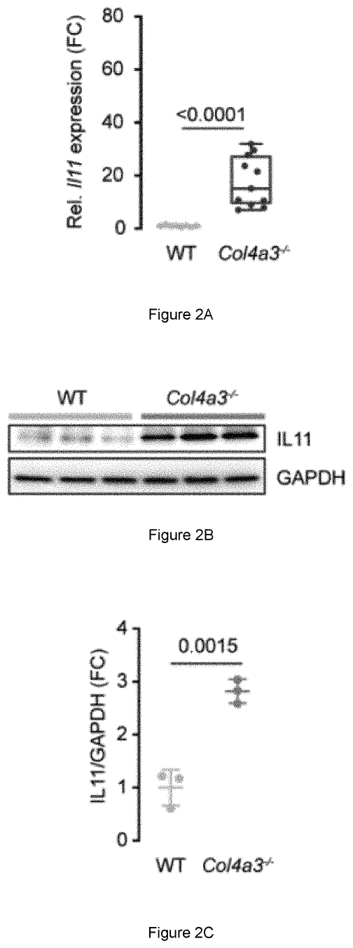

The results of the experiment are shown in FIGS. 1A and 1B. Treatment of mice from 6 weeks of age with Enx203 was found to increase survival by up to 40% relative to survival of mice treated with isotype-matched control antibody.

Thus the inventors establish antagonism of IL-11-mediated signalling as a useful therapeutic intervention for Alport syndrome.

This is the first therapeutic intervention found to be effective to increase survival of mice in this model when commenced later than 4 weeks after birth.

### Example 2: A Neutralising Antibody Antagonist of IL-11-Mediated Signalling Improves Renal Function and Increases Lifespan in a Mouse Model of Alport Syndrome

2.1 Overview

Background: Alport syndrome is a genetic disorder characterized by a defective glomerular basement membrane, tubulointerstitial fibrosis and progressive renal failure. The role of interleukin 11 (IL11) in Alport syndrome is unknown.

Methods: The effects of a neutralizing IL11 antibody (X203) were assessed, as compared to IgG control, in Col4a3^{-/-} mice, from six weeks of age, on lifespan, renal tubule damage, function, fibrosis, and inflammation using histology, qPCR, immunoblotting and immunohistochemistry.

Results: Renal expression of IL11 is elevated in Col4a3^{-/-} mice and the IL11RA1 receptor is found predominantly in tubular cells and podocytes. Administration of X203 reduced albuminuria and improved renal function, assessed by BUN and serum creatinine levels. X203 also extended the median life span of Col4a3^{-/-} mice by 41.6%, from 62.5 to 88.5 days. Podocyte numbers and levels of key podocytes proteins including Wilms' Tumor 1 and Podocin, which are reduced in Col4a3^{-/-} mice, were restored towards normal by X203 administration. The beneficial effects of X203 on kidney structure and function were accompanied by reduced severity of renal fibrosis and inflammation, markers of tubule damage, and features of epithelial-to-mesenchymal transition. Pathogenic ERK and STAT3 activities were elevated in Col4a3^{-/-} mice and these pathways were largely reduced by administration of X203.

Conclusion: In a mouse model of Alport syndrome, IL11 is upregulated in the kidney and a neutralizing IL11 antibody, given at a time point when angiotensin-converting enzyme inhibition is ineffective, improves kidney structure and function while extending lifespan.

2.2 Materials and Methods

Antibodies

ACTA2 (19245, CST; WB), Cyclin D1 (55506, CST), E-Cadherin (3195, CST), p-ERK1/2 (4370, CST), ERK1/2 (4695, CST), GAPDH (2118, CST), IgG (11E10, Aldevron; which is produced from 1.10E+11 cells (ATCC, No. CRL-1907)), neutralizing anti-IL11 (X203, Aldevron), anti-IL11RA (X209, Aldevron), NHPS2/Podocin (ab181143, Abcam), PCNA (13110, CST), SNAIL (3879, CST, WB), p-STAT3 (4113, CST), STAT3 (4904, CST), TGFβ (3711, CST), Wilms' Tumor 1 (ab89901, Abcam, IHC, Wilms' Tumor 1 (ab267377, Abcam, WB), anti-rabbit HRP (7074, CST), anti-mouse HRP (7076, CST).

Mouse Model of Alport

Animal studies were carried out in compliance with the recommendations in the Guidelines on the Care and Use of Animals for Scientific Purposes of the National Advisory Committee for Laboratory Animal Research (NACLAR). All experimental procedures were approved (SHS/2019/1482) and conducted in accordance with the SingHealth Institutional Animal Care and Use Committee.

Col4a3^{-/-} (Col4a3^{tm1Dec}) mice were purchased from The Jackson Laboratory (https://www.jax.org/strain/002908). Mice were housed in temperatures of 21-24° C. with 40-70% humidity on a 12 h light/12 h dark cycle and provided with food and water ad libitum. For treatment study, Col4a3^{-/-} were administered 20 mg/kg of anti-IL11 (X203) or IgG isotype control (11E10) by intraperitoneal (IP) injection starting from 6 weeks of age twice a week for 2.5 weeks; wild-type littermates were used as controls. Mice were sacrificed for blood and kidney collection when they were 8.5-week-old. For lifespan study, IgG/X203 was administered to 6-week-old Col4a3^{-/-} (2×/week) until death ensued.

Western Blot

Western blot was carried out on total protein extracts from mouse kidney tissues. Kidneys were lysed in radioimmunoprecipitation assay (RIPA) buffer containing protease and phosphatase inhibitors (Thermo Scientifics), followed by centrifugation to clear the lysate. Protein concentrations were determined by Bradford assay (Bio-Rad). Protein lysates were separated by SDS-PAGE, transferred to PVDF membrane, and subjected to immunoblot analysis for various antibodies as outlined in the main text, figures, or and/or figure legends. Proteins were visualized using the ECL detection system (Pierce) with the appropriate secondary antibodies: anti-rabbit HRP or antimouse HRP.

Quantitative Polymerase Chain Reaction (qPCR)

Total RNA was extracted from snap-frozen kidney tissues using Trizol (Invitrogen) followed by RNeasy column (Qiagen) purification. cDNAs were synthesized with iScript™ cDNA synthesis kit (Bio-Rad) according to manufacturer's instructions. Gene expression analysis was performed on duplicate samples with either TaqMan (Applied Biosystems) or fast SYBR green (Qiagen) technology using StepOnePlus™ (Applied Biosystem) over 40 cycles. Expression data were normalized to GAPDH mRNA expression and fold change was calculated using 2^{-ΔΔct} method. The sequences of specific TaqMan probes and SYBR green primers are available upon request.

Colorimetric Assays

The levels of blood urea nitrogen (BUN) and creatinine in mouse serum were measured using Urea Assay Kit (ab83362, Abcam) and Creatinine Assay Kit (ab65340, Abcam), respectively. Urine albumin and creatinine levels were measured using Mouse Albumin ELISA kit (ab108792, Abcam) and Creatinine Assay Kit (ab204537, Abcam), respectively. All ELISA and colorimetric assays were performed according to the manufacturer's protocol.

Masson Trichrome's Staining

Kidney tissues were fixed for 48 hours at RT in 10% neutral-buffered formalin (NBF), dehydrated, embedded in paraffin, and sectioned at 7 μm. Transverse kidney sections were then stained with Masson's Trichrome according to standard protocol. Images of the sections were captured by light microscopy and blue-stained fibrotic areas were semi-quantitatively determined with Image-J software (color deconvolution-Masson Trichrome) from the whole kidney area (100× field, n=4 kidneys/group). Treatment and genotypes were not disclosed to investigators performing the histology and generating semi-quantitative readouts.

Immunohistochemistry

Kidneys were fixed in 10% neutral-buffered formalin (NBF), paraffinized, cut into 7 μm sections, incubated with primary antibodies overnight and visualized using an ImmPRESS HRP anti-rabbit IgG polymer detection kit (MP-7401, Vector Laboratories) with ImmPACT DAB Peroxidase Substrate (SK-4105, Vector Laboratories). Quantification of WT^{+ve} cells were performed in a blinded fashion from 4 images (200× field)/kidney (n=3-4 kidneys/group).

Statistical Analyses

Statistical analyses were performed using GraphPad Prism software (version 8). Statistical significance between control and experimental groups were analysed by twosided Student's t tests or by one-way ANOVA as indicated in the figure legends. P values were corrected for multiple testing according to Tukey when several conditions were compared to each other within one experiment. Comparison analysis for two parameters from two different groups were performed by two-way ANOVA. Survival curves were analyzed by Gehan-Breslow-Wilcoxon test. The criterion for statistical significance was P<0.05.

2.3 Results

IL11 is Upregulated in the Kidneys of Col4a3^{-/-} Mice.

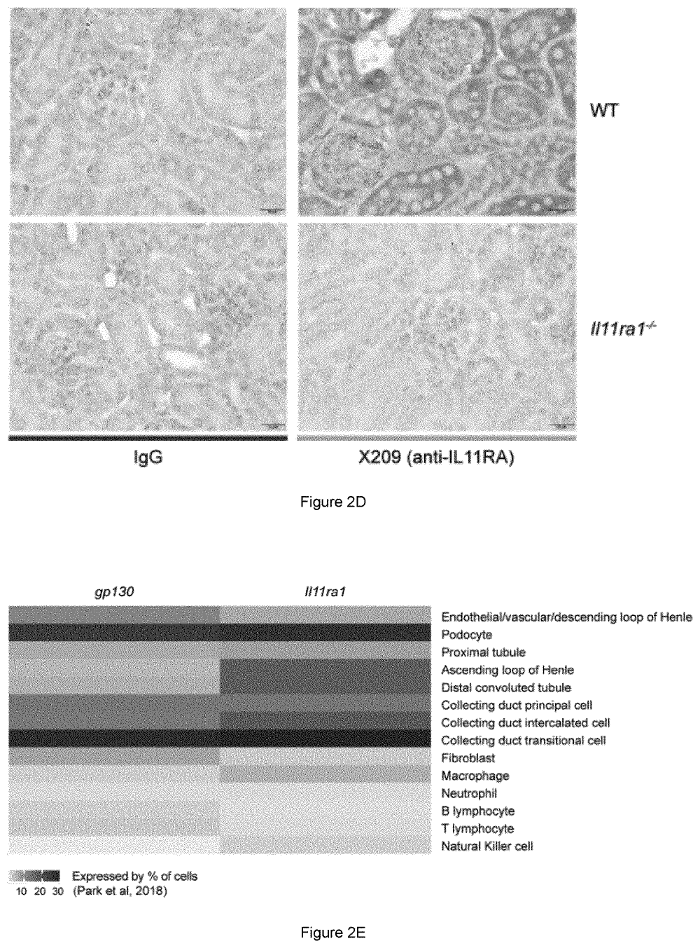

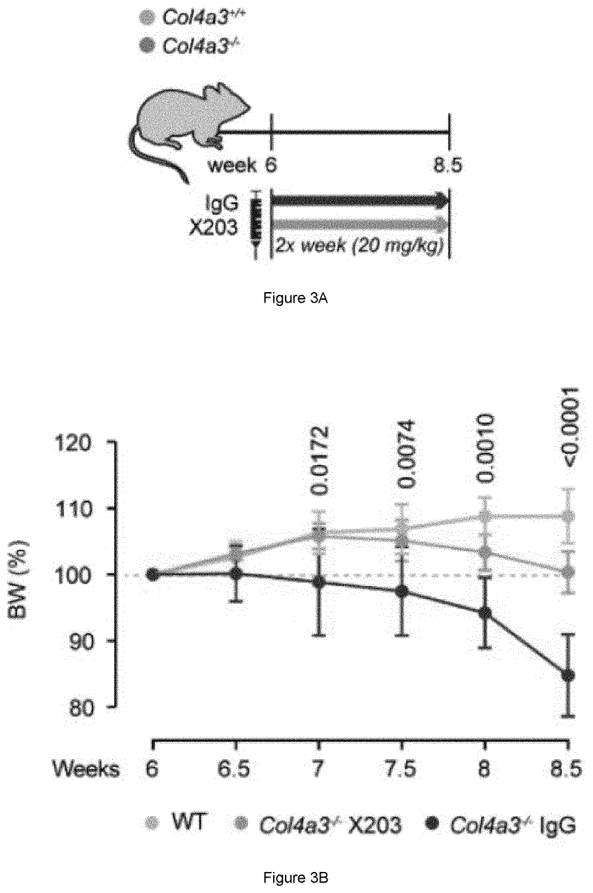

IL11 is not expressed in normal healthy tissues but its induction is commonly seen in fibroinflammatory diseases¹⁵. Expression of II11 mRNA was profiled in kidneys of Col4a3^{-/-} mice, and was found to be upregulated (17.8-fold, P<0.0001), as compared to wild-type littermate controls (FIG. 2A). IL11 was also notably upregulated (P<0.0015) at the protein level (FIGS. 2B to 2C).

Stromal cells, epithelial cells, and other cells can express IL11, and so the cells in the kidney that express the IL11 receptor (IL11RA1) were determined by both immunohistochemistry and mining publicly available single cell RNA sequencing (scRNA-seq) data¹⁶. In wild-type mice, IL11RA1 expression was easily seen in tubules and also in the glomerulus, whereas no staining was seen in sections from II11ra1^{-/-} mice, confirming specificity of detection (FIG. 2D). In scRNA-seq data from wild-type mice¹⁶, it was observed that II11ra1 and its partner receptor (gp130) were most highly expressed in podocytes and collecting ducts with lesser expression in tubule cells across the nephron, as well as in fibroblasts (FIG. 2E).

Antibody Neutralization of IL11 Reduces Molecular Pathologies in Col4a3^{-/-} Mice.

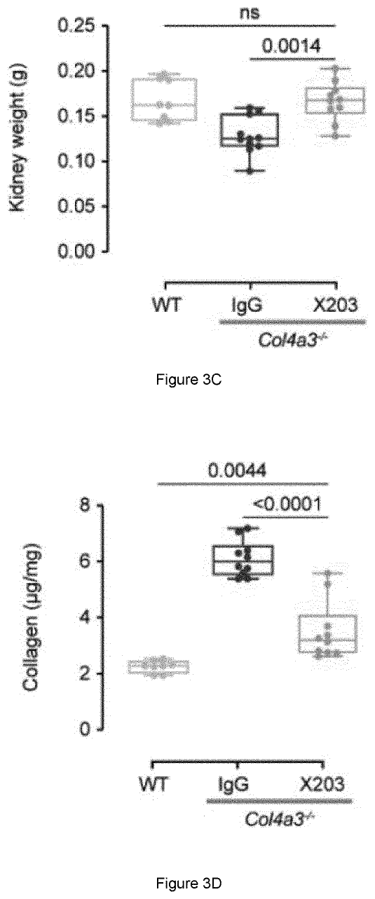

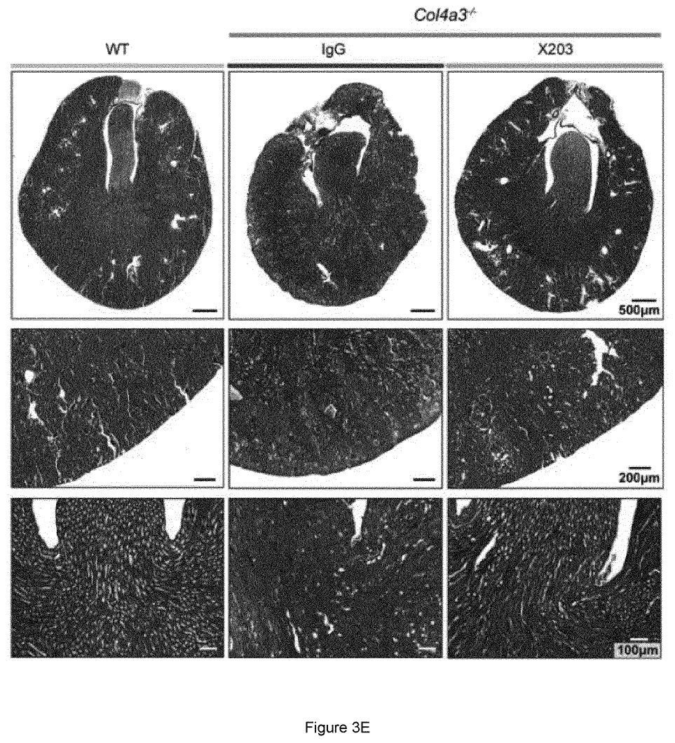

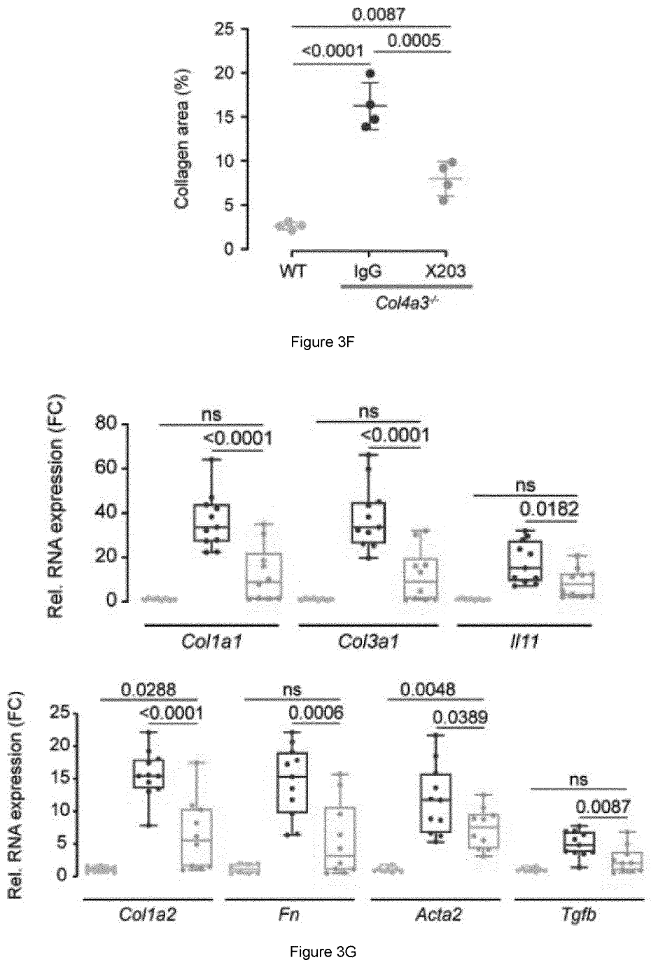

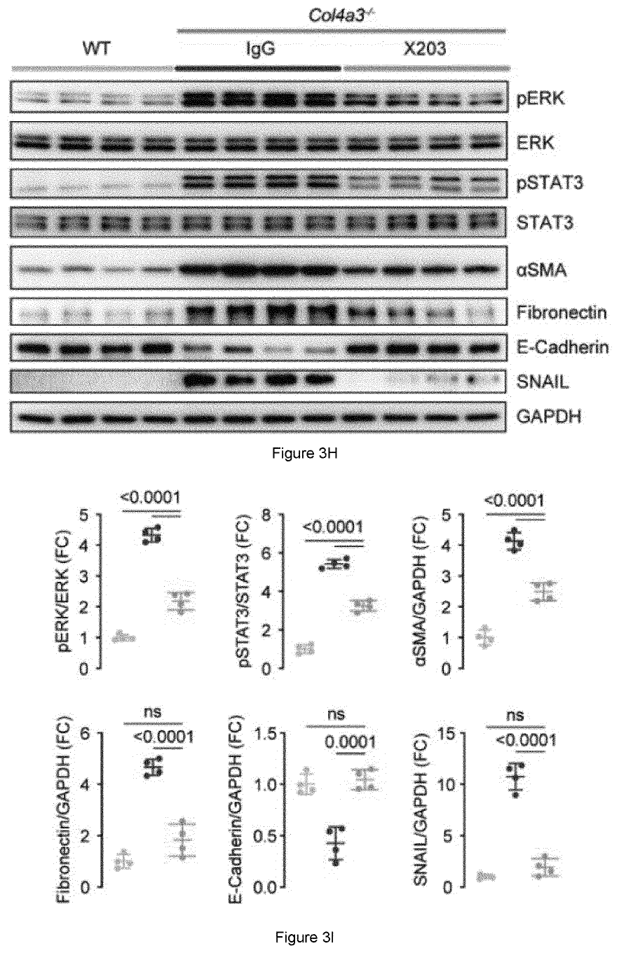

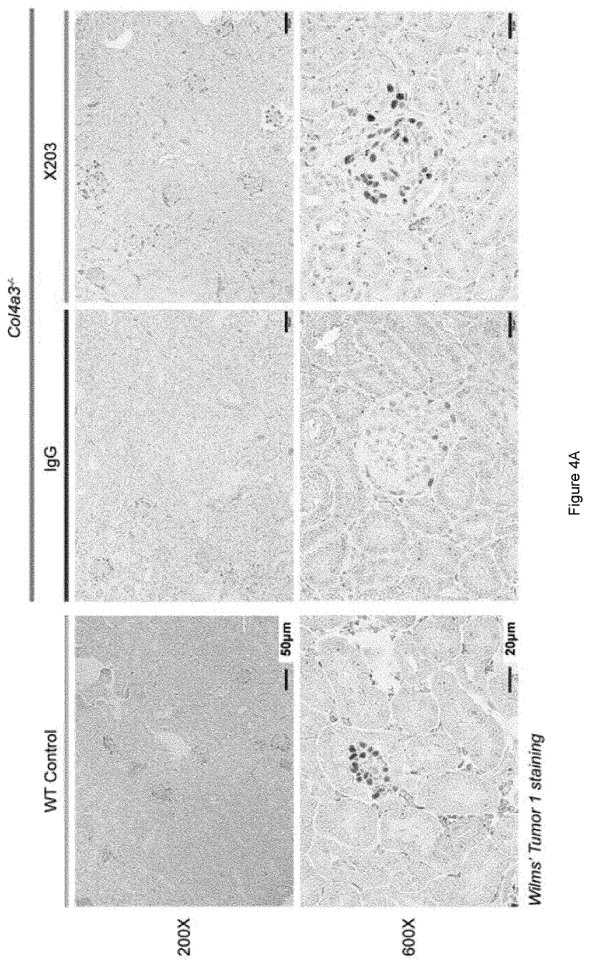

Over recent years, antibodies that inhibit IL11 signaling in mouse and human cells have been developed^{16,17}. One of these neutralizing IL11 antibodies (X203) or an IgG control were administered to 6-week-old Col4a3^{-/-} mice, the time point when initiation of ramipril has proven ineffective⁶, and examined renal pathologies at 8.5 weeks of age, as compared to age-matched wild-type controls (FIG. 3A).

At the end of the study period, total body weight loss, measured as a percentage of their starting body weight, was significantly attenuated in Col4a3^{-/-} mice receiving X203, compared to those administered with IgG (IgG: 30%, X203:17%; P=0.0003) (FIG. 3B). As compared to Col4a3^{-/-} mice receiving IgG, X203-treated mice exhibited preserved kidney mass (FIG. 3C) and had significantly less kidney fibrosis by both biochemical and histological assessments (FIGS. 3D to 3F). Gene expression analyses showed renal levels of extracellular matrix genes (Col1a1, Col1a2, Col3a1 and Fn), the myofibroblast marker Acta2 and pro-fibrotic factors (II11 and Tgfb1) were all reduced by X203 as compared to IgG (FIG. 3G). The effect seen on transcript expression was confirmed at the protein level for alpha-smooth muscle actin (αSMA) and fibronectin (FIGS. 3H and 3I).

At the signaling level, IL11 is known to activate ERK across primary cell types and this pathway has been mechanistically linked with MI-driven fibrosis¹⁷⁻¹⁹. IL11 inhibition in vivo can also be associated with reduced STAT3 activation, which is thought to be a secondary phenomenon reflecting lesser stromal-driven inflammation^{17,20,21.} As compared to wild-type mice, kidneys from Col4a3^{-/-} mice treated with IgG exhibited elevated ERK and STAT3 activation, in contrast ERK and STAT3 phosphorylation was largely diminished in kidneys of X203-treated Col4a3^{-/-} mice (FIGS. 3H and 3I). These data are consistent with X203 target engagement in the kidney, reduced ERK activation and diminished inflammation.

In many kidney diseases, it is thought that damaged TECs undergo a pEMT process, which is critical for the subsequent development of tubulointerstitial fibrosis and CKD^{22,23}. TEC pEMT is characterised by increased SNAI1 expression and reciprocal downregulation of E-Cadherin, which is regulated in part by TGFβ22,23. As compared to wild-type controls, Col4a3^{-/-} mice receiving IgG exhibited a strong molecular signature of EMT with increased SNAI1 and decreased E-Cadherin expression (FIGS. 3H and 3I). In contrast, SNAI1 and E-Cadherin levels in Col4a3^{-/-} mice receiving X203 were similar to those seen in wild-type mice.

Podocyte Preservation and Lesser Renal Inflammation is Associated with Inhibition of IL11 Signaling in Col4a3^{-/-} Mice.

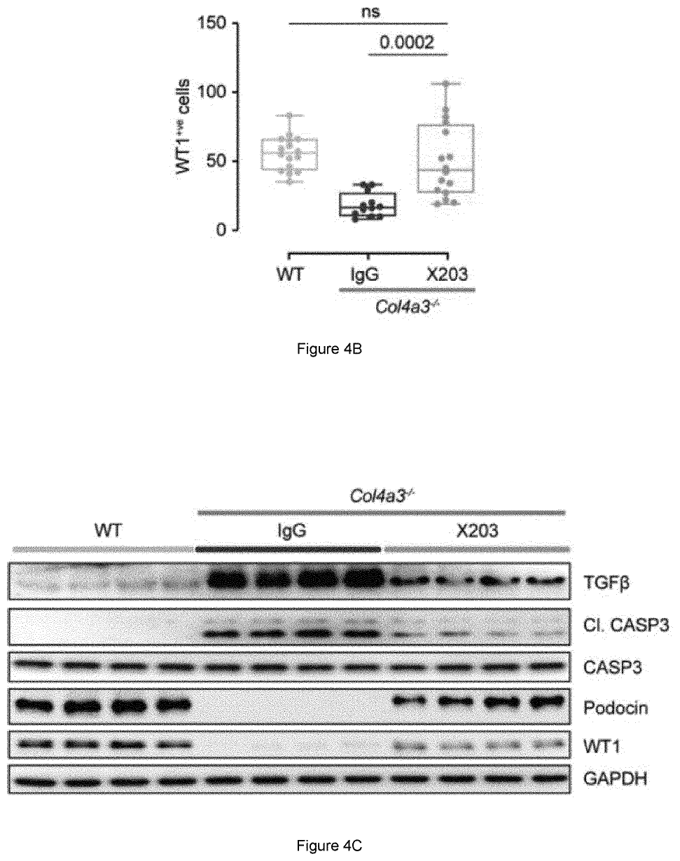

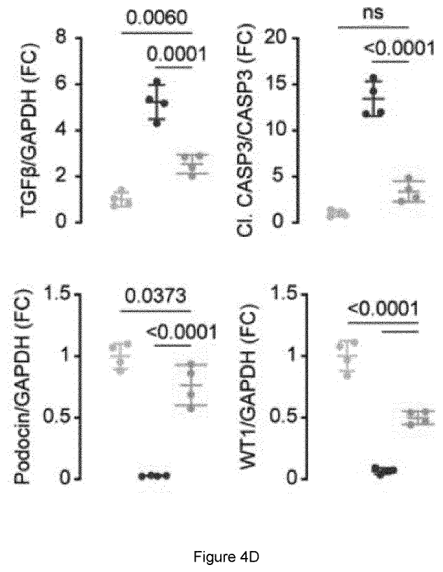

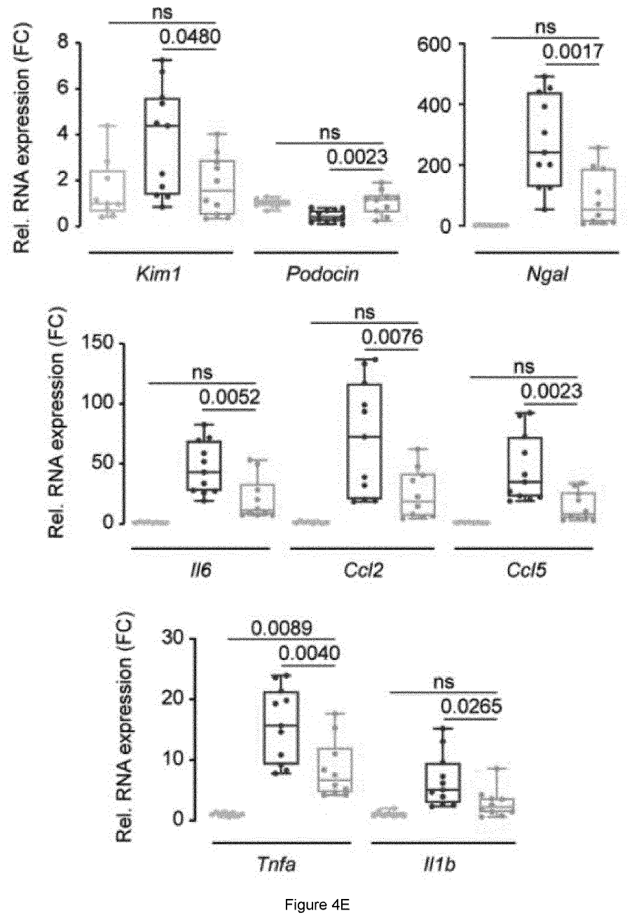

AS affects GBM composition leading to podocyte dysfunction/loss that relates to TGFβ activity in both podocytes and TECs⁹. Immunohistochemistry analysis of the podocyte marker, Wilms' Tumor 1 (WT1) revealed a greater staining in wild-type mice and X203-treated Col4a3^{-/-} mice, as compared to IgG-treated Col4a3^{-/-} mice (FIG. 4A). Quantification of the number of WT1-positive cells (podocytes) was carried out in a blinded fashion and confirmed significant (P=0.0002) restoration of podocytes in Col4a3^{-/-} mice receiving X203 as compared to Col4a3^{-/-} mice receiving IgG (FIG. 4B). Preservation of podocytes in X203-treated Col4a3^{-/-} mice was further confirmed by immunoblotting and findings were extended using Podocin, a second podocyte marker (FIGS. 4C and 4D).

TGFβ upregulation in podocytes and tubular cells, which is coincident with the onset of proteinuria in the Col4a3^{-/-} mouse^{9,24}, is thought of importance for disease pathogenesis in AS. Levels of TGFβ were therefore examined, and it was observed that X203, but not IgG, significantly reduced the degree of TGFβ upregulation in the kidneys of Col4a3^{-/-} mice (FIGS. 4C and 4D). Apoptosis of podocytes and tubule cells is implicated in AS and caspase activity is reduced in Col4a3^{-/-} mice given Olmesartan²⁴. Caspase 3 activation was observed in the IgG-treated Col4a3^{-/-} mice that was reduced by X203 administration (FIGS. 4C and 4D).

Tnfα expression in podocytes is of particular importance in AS and leads to podocyte apoptosis and glomerulosclerosis¹³. It was therefore notable that X203 reduced Tnfα expression in Col4a3^{-/-} mice, as compared to IgG treated controls (FIG. 4E). Markers of tubule damage and inflammation were also assessed. As compared to wild-type mice, control Col4a3^{-/-} mice had elevated indicators of tubule damage (Kim1 and Ngal), which were restored by X203 administration towards the levels seen in wildtype mice (FIG. 4E). Proinflammatory interleukins (II6 and II1b) and CC chemokines (CcI2 and CcI5) were also elevated in Col4a3^{-/-} mice receiving IgG and were equally diminished by administration of X203 (FIG. 4E).

Inhibition of IL11 Signaling Improves Renal Function and Prolongs Lifespan in Col4a3^{-/-} Mice.

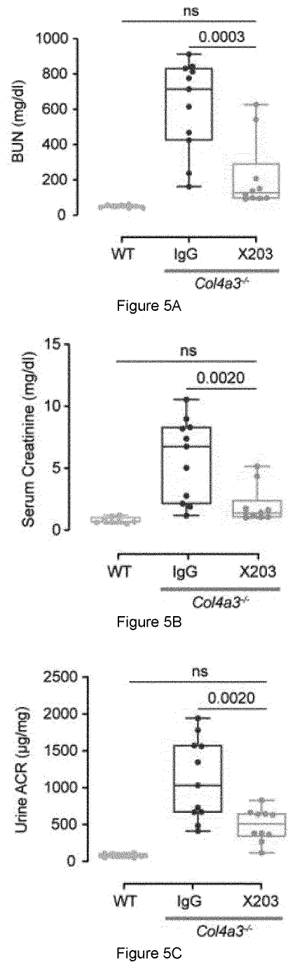

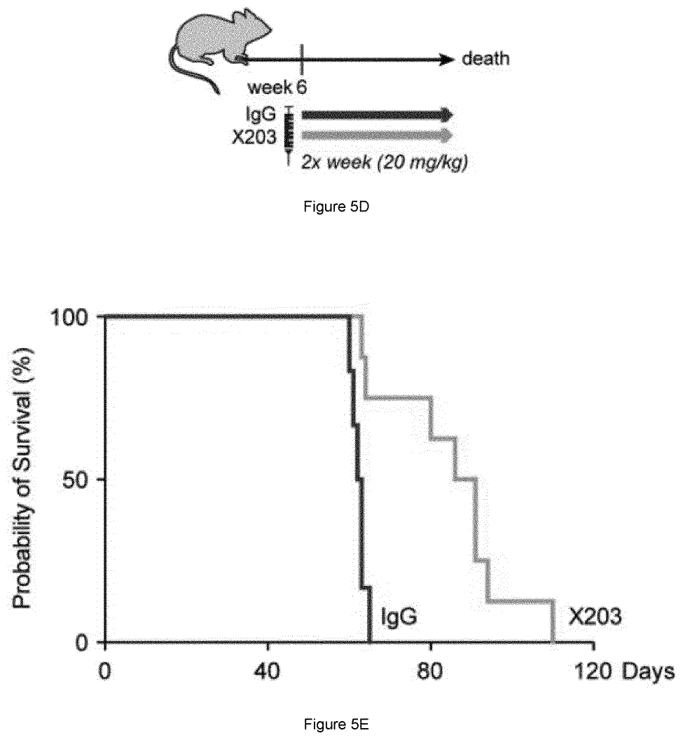

Next, it was investigated whether inhibition of IL11 signaling, which mitigated intermediate phenotypes of renal pathology in Col4a3^{-/-} mice, also improved renal function. To do so serum blood urea nitrogen (BUN) and creatinine (Cr) and also urinary albumin:creatinine ratios were determined at study end (8.5 weeks of age). As compared to wild-type mice, IgG-treated Col4a3^{-/-} mice had elevated BUN, Cr and urinary ACR levels (fold elevation compared to wild-type: 12.4, 7.3, 13.6, respectively), whereas administration of X203 from week 6 markedly lowered BUN, Cr and urinary ACR consistent with an overall improvement in renal function (FIGS. 5A to 5C).

Death from progressive kidney failure is consistently seen in untreated Col4a3^{-/-} mice from 8.5 weeks of age and mean survival times are reproducibly reported at around 10 weeks (e.g. 71 days⁶; 69 days²⁶). The Col4a3^{-/-} mice used for the studies described here had a similar mean survival of 62.7±1.9 days, albeit with a trend towards a shorter lifespan. It was investigated whether administration of X203 (20 mg/kg; 2×/week) to 6-week old Col4a3^{-/-} mice extended life, as compared to IgG control-treated mice (FIG. 5D). This proved to be the case and X203 extended median lifespan from 62.5 to 88.5 days (P=0.0015) (FIG. 5E).

2.4 Discussion

While ACEi is the mainstay of therapy for patients with AS, progression to endstage renal failure is typical^{6,10}. This shortcoming likely reflects the complex renal pathology in AS, involving GBM-specific initiating factors and generic tubulointerstitial disease mechanisms. ACEi may impact a number of AS pathologies and reducing ultrafiltration is thought of as of primary importance. As IL11 antibody therapy is beneficial at 6 weeks of age in Col4a3^{-/-} mice, when ACEi is ineffective, this suggests an alternative mechanism of action is associated with inhibition of IL11.

IL11 is secreted from a variety of stromal and epithelial cells in response to cellular injury and acts in an autocrine and paracrine manner to cause epithelial cell dysfunction, stromal activation and tissue inflammationm. In the kidney, IL11RA is expressed on tubule cells throughout the nephron and in podocytes, two cell types that can be affected by pEMT^{22,23,26}. One way that inhibition of IL11 signaling is protective in Col4a3^{-/-} mice could be through inhibition of pEMT, thus preserving both podocyte and TEC function. This could be secondary to lower TGFβ, a master determinant of pEMT in the kidney^{22,23}, after X203 administration. While inhibition of TGFβ directly is proinflammatory²⁷ it is shown here in the kidney, as in other tissues, that inhibition of IL11 is anti-inflammatory^{17,21,28.}

The results suggest that inhibition of IL11 signaling is a plausible therapeutic approach for patients with AS and perhaps other forms of progressive CKD, especially as new IL11-targeting therapies are progressing towards the clinic. Besides limited developmental bone/tooth defects, humans with loss-of-function in IL11RA are otherwise well with normal immune function and lifespans, as are II11ra1 null mice. Furthermore, two recently and separately developed II11 null mice appear normal with no bone deficits^{29,30} and it could be that inhibiting the IL11 cytokine has advantages over targeting IL11RA. Taken together, the human and mouse 1L11RA and II11 genetic null data, along with long-term antibody studies in mice¹⁷, provide a safety signal needed to consider long term trials of IL11 inhibition in AS^{16,31}.

### REFERENCES

1. Hudson B G, Tryggvason K, Sundaramoorthy M, Neilson E G: Alport's Syndrome, Goodpasture's Syndrome, and Type IV Collagen [Internet]. New England Journal of Medicine. 348: 2543-2556, 2003 Available from: http://dx.doi.org/10.1056/nejmra022296

2. Hudson B G, Reeders S T, Tryggvason K: Type IV collagen: structure, gene organization, and role in human diseases. Molecular basis of Goodpasture and Alport syndromes and diffuse leiomyomatosis. J. Biol. Chem. 268: 26033-26036, 1993
3. Savige J: Alport syndrome: its effects on the glomerular filtration barrier and implications for future treatment. J. Physiol. 592: 4013-4023, 2014
4. Funk S D, Lin M-H, Miner J H: Alport syndrome and Pierson syndrome: Diseases of the glomerular basement membrane. Matrix Biol. 71-72: 250-261, 2018
5. Jais J P, Knebelmann B, Giatras I, De Marchi M, Rizzoni G, Renieri A, Weber M, Gross O, Netzer K O, Flinter F, Pirson Y, Verellen C, Wieslander J, Persson U, Tryggvason K, Martin P, Hertz J M, Schröder C, Sanak M, Krejcova S, Carvalho M F, Saus J, Antignac C, Smeets H, Gubler M C: X-linked Alport syndrome: natural history in 195 families and genotype-phenotype correlations in males. J. Am. Soc. Nephrol. 11: 649-657, 2000
6. Gross O, Beirowski B, Koepke M-L, Kuck J, Reiner M, Addicks K, Smyth N, Schulze-Lohoff E, Weber M: Preemptive ramipril therapy delays renal failure and reduces renal fibrosis in COL4A3-knockout mice with Alport syndrome. Kidney Int 63: 438-446, 2003
7. Gross O, Tönshoff B, Weber L T, Pape L, Latta K, Fehrenbach H, Lange-Sperandio B, Zappel H, Hoyer P, Staude H, König S, John U, Gellermann J, Hoppe B, Galiano M, Hoecker B, Ehren R, Lerch C, Kashtan C E, Harden M, Boeckhaus J, Friede T, German Pediatric Nephrology (GPN) Study Group and EARLY PRO-TECT Alport Investigators: A multicenter, randomized, placebo-controlled, double-blind phase 3 trial with open-arm comparison indicates safety and efficacy of nephroprotective therapy with ramipril in children with Alport's syndrome. Kidney Int. 97: 1275-1286, 2020
8. Gross O, Licht C, Anders H J, Hoppe B, Beck B, Tonshoff B, Hocker B, Wygoda S, Ehrich J H H, Pape L, Konrad M, Rascher W, Dotsch J, Müller-Wiefel D E, Hoyer P, Study Group Members of the Gesellschaft für Pädiatrische Nephrologie, Knebelmann B, Pirson Y, Grunfeld J-P, Niaudet P, Cochat P, Heidet L, Lebbah S, Torra R, Friede T, Lange K, Müller G A, Weber M: Early angiotensin-converting enzyme inhibition in Alport syndrome delays renal failure and improves life expectancy. Kidney Int 81: 494-501, 2012
9. Sayers R, Kalluri R, Rodgers K D, Shield C F, Meehan D T, Cosgrove D: Role for transforming growth factor-beta1 in alport renal disease progression. Kidney Int 56: 1662-1673, 1999
10. Kashtan C E: Renal transplantation in patients with Alport syndrome: patient selection, outcomes, and donor evaluation. Int. J. Nephroi. Renovasc. Dis. 11: 267-270, 2018
11. Hood J C, Dowling J, Bertram J F, Young R J, Huxtable C, Robinson W, Savige J: Correlation of histopathological features and renal impairment in autosomal dominant Alport syndrome in Bull terriers. Nephrol. Dial. Transplant 17: 1897-1908, 2002
12. Qi R, Yang C: Renal tubular epithelial cells: the neglected mediator of tubulointerstitial fibrosis after injury. Cell Death Dis. 9: 1126, 2018
13. Ryu M, Mulay S R, Miosge N, Gross O, Anders H-J: Tumour necrosis factor-α drives Alport glomerulosclerosis in mice by promoting podocyte apoptosis. J. Pathol. 226: 120-131, 2012
14. Schafer S, Viswanathan S, Widjaja A A, Lim W-W, Moreno-Moral A, DeLaughter D M, Ng B, Patone G, Chow K, Khin E, Tan J, Chothani S P, Ye L, Rackham O J L, Ko NSJ, Sahib N E, Pua C J, Zhen N T G, Xie C, Wang M, Maatz H, Lim S, Saar K, Blachut S, Petretto E, Schmidt S, Putoczki T, Guimarães-Camboa N, Wakimoto H, van Heesch S, Sigmundsson K, Lim S L, Soon J L, Chao V T T, Chua Y L, Tan T E, Evans S M, Loh Y J, Jamal M H, Ong K K, Chua K C, Ong B-H, Chakaramakkil M J, Seidman J G, Seidman C E, Hubner N, Sin K Y K, Cook S A: IL-11 is a crucial determinant of cardiovascular fibrosis. Nature 552: 110-115, 2017
15. Cook S A, Schafer S: Hiding in Plain Sight: Interleukin-11 Emerges as a Master Regulator of Fibrosis, Tissue Integrity, and Stromal Inflammation. Annu. Rev. Med. 71: 263-276, 2020
16. Park J, Shrestha R, Qiu C, Kondo A, Huang S, Werth M, Li M, Barasch J, Susztak K: Single-cell transcriptomics of the mouse kidney reveals potential cellular targets of kidney disease. Science [Internet] 2018 Available from: http://dx.doi.org/10.1126/science.aar2131
17. Widjaja A A, Singh B K, Adami E, Viswanathan S, Dong J, D'Agostino G A, Ng B, Lim W W, Tan J, Paleja B S, Tripathi M, Lim S Y, Shekeran S G, Chothani S P, Rabes A, Sombetzki M, Bruinstroop E, Min L P, Sinha R A, Albani S, Yen P M, Schafer S, Cook S A: Inhibiting Interleukin 11 Signaling Reduces Hepatocyte Death and Liver Fibrosis, Inflammation, and Steatosis in Mouse Models of Nonalcoholic Steatohepatitis. Gastroenterology 157: 777-792.e14, 2019
18. Lim W-W, Corden B, Ye L, Viswanathan S, Widjaja A A, Xie C, Su L, Tee N G Z, Schafer S, Cook S A: Antibody-mediated neutralization of IL11 signalling reduces ERK activation and cardiac fibrosis in a mouse model of severe pressure overload. Clin. Exp. Pharmacol. Physiol. [Internet] 2021 Available from: http://dx.doi.org/10.1111/1440-1681.13458
19. Adami E, Viswanathan S, Widjaja A A, Ng B, Chothani S, Zhihao N, Tan J, Lio P M, George B L, Altunoglu U, Ghosh K, Paleja B S, Schafer S, Reversade B, Albani S, Ling A L H, O'Reilly S, Cook S A: IL11 is elevated in systemic sclerosis and IL11-dependent ERK signaling underlies TGFβ-mediated activation of dermal fibroblasts. Rheumatology [Internet] 2021 Available from: http://dx.doi.org/10.1093/rheumatology/keab168
20. Dong J, Viswanathan S, Adami E, Singh B K, Chothani S P, Ng B, Lim V W V, Zhou J, Tripathi M, Ko N S J, Shekeran S G, Tan J, Lim S Y, Wang M, Lio P M, Yen P M, Schafer S, Cook S A, Widjaja A A: Hepatocyte-specific IL11 cis-signaling drives lipotoxicity and underlies the transition from NAFLD to NASH. Nat. Commun. 12: 66, 2021
21. Ng B, Dong J, Viswanathan S, Widjaja A A, Paleja B S, Adami E, Ko N S J, Wang M, Lim S, Tan J, Chothani S P, Albani S, Schafer S, Cook S A: Fibroblast-specific IL11 signaling drives chronic inflammation in murine fibrotic lung disease. FASEB J. [Internet] 2020 Available from: http://dx.doi.org/10.1096/fj.202001045RR
22. Lovisa S, LeBleu V S, Tampe B, Sugimoto H, Vadnagara K, Carstens J L, Wu C-C, Hagos Y, Burckhardt B C, Pentcheva-Hoang T, Nischal H, Allison J P, Zeisberg M, Kalluri R: Epithelial-to-mesenchymal transition induces cell cycle arrest and parenchymal damage in renal fibrosis. Nat. Med. 21: 998-1009, 2015
23. Grande M T, Sanchez-Laorden B, Lopez-Blau C, De Frutos C A, Boutet A, Arevalo M, Rowe R G, Weiss S J, Lopez-Novoa J M, Nieto M A: Snaill-induced partial epithelial-to-mesenchymal transition drives renal fibrosis in mice and can be targeted to reverse established disease. Nat. Med. 21: 989-997, 2015
24. Suh S H, Choi H S, Kim C S, Kim I J, Ma S K, Scholey J W, Kim S W, Bae E H: Olmesartan Attenuates Kidney Fibrosis in a Murine Model of Alport Syndrome by Suppressing Tubular Expression of TGFβ. Int. J. Mol. Sci. [Internet] 20: 2019 Available from: http://dx.doi.org/10.3390/ijms20153843
25. Ninichuk V, Gross O, Reichel C, Khandoga A, Pawar R D, Ciubar R, Segerer S, Belemezova E, Radomska E, Luckow B, Perez de Lema G, Murphy P M, Gao J-L, Henger A, Kretzler M, Horuk R, Weber M, Krombach F, Schlondörff D, Anders H-J: Delayed chemokine receptor 1 blockade prolongs survival in collagen 4A3-deficient mice with Alport disease. J. Am. Soc. Nephroi. 16: 977-985, 2005
26. Ying Q, Wu G: Molecular mechanisms involved in podocyte EMT and concomitant diabetic kidney diseases: an update. Ren. Fail. 39: 474-483, 2017 27. Shull M M, Ormsby I, Kier A B, Pawlowski S, Diebold R J, Yin M, Allen R, Sidman C, Proetzel G, Calvin D: Targeted disruption of the mouse transforming growth factorbeta 1 gene results in multifocal inflammatory disease. Nature 359: 693-699, 1992
28. Lim W-W, Corden B, Ng B, Vanezis K, D'Agostino G, Widjaja A A, Song W-H, Xie C, Su L, Kwek X-Y, Tee N G Z, Dong J, Ko N S J, Wang M, Pua C J, Jamal M H, Soh B, Viswanathan S, Schafer S, Cook S A: Interleukin-11 is important for vascular smooth muscle phenotypic switching and aortic inflammation, fibrosis and remodeling in mouse models [Internet]. Scientific Reports. 10: 2020 Available from: http://dx.doi.org/10.1038/s41598-020-74944-7
29. Nishina T, Deguchi Y, Ohshima D, Takeda W, Ohtsuka M, Shichino S, Ueha S, Yamazaki S, Kawauchi M, Nakamura E, Nishiyama C, Kojima Y, Adachi-Akahane S, Hasegawa M, Nakayama M, Oshima M, Yagita H, Shibuya K, Mikami T, Inohara N, Matsushima K, Tada N, Nakano H: Interleukin-11-expressing fibroblasts have a unique gene signature correlated with poor prognosis of colorectal cancer. Nat. Commun. 12: 2281, 2021
30. Ng B, Widjaja A A, Viswanathan S, Dong J: Similarities and differences between IL11 and IL11RA1 knockout mice for lung fibro-inflammation, fertility and craniosynostosis. bioRxiv [Internet] 2020 Available from: https://www.biorxiv.org/content/10.1101/2020.12.10.420695v1.abstract
31. Widjaja A A, Chothani S P, Cook S A: Different roles of interleukin 6 and interleukin 11 in the liver: implications for therapy. Hum. Vaccin. Immunother. 16: 2357-2362, 2020

1. An agent capable of inhibiting interleukin 11 (IL-11)-mediated signalling for use in a method of treating or preventing Alport syndrome.

2. Use of an agent capable of inhibiting interleukin 11 (IL-11)-mediated signalling in the manufacture of a medicament for use in a method of treating or preventing Alport syndrome.

3. A method of treating or preventing Alport syndrome, comprising administering a therapeutically or prophylactically effective amount of an agent capable of inhibiting interleukin 11 (IL-11)-mediated signalling to a subject.

4. The agent for use according to claim 1, the use according to claim 2, or the method according to claim 3, wherein the agent is an agent capable of preventing or reducing the binding of interleukin 11 (IL-11) to a receptor for interleukin 11 (IL-11R).

5. The agent for use according to claim 1 or claim 4, the use according to claim 2 or claim 4, or the method according to claim 3 or claim 4, wherein the agent is capable of binding to interleukin 11 (IL-11) or a receptor for interleukin 11 (IL-11R).

6. The agent for use according to any one of claim 1, 4 or 5, the use according to any one of claim 2, 4 or 5, or the method according to any one of claims 3 to 5, wherein the agent is selected from the group consisting of: an antibody or an antigen-binding fragment thereof, a polypeptide, a peptide, a nucleic acid, an oligonucleotide, an aptamer or a small molecule.

7. The agent for use, the use or the method according to claim 5 or claim 6, wherein the agent is an antibody or an antigen-binding fragment thereof.

8. The agent for use, the use or the method according to claim 7, wherein the agent is an anti-IL-11 antibody antagonist of IL-11-mediated signalling, or an antigen-binding fragment thereof.

9. The agent for use, the use or the method according to claim 7 or claim 8, wherein the antibody or antigen-binding fragment comprises:
(i) a heavy chain variable (VH) region incorporating the following CDRs:
HC-CDR1 having the amino acid sequence of SEQ ID NO:34
HC-CDR2 having the amino acid sequence of SEQ ID NO:35
HC-CDR3 having the amino acid sequence of SEQ ID NO:36; and
(ii) a light chain variable (VL) region incorporating the following CDRs:
LC-CDR1 having the amino acid sequence of SEQ ID NO:37
LC-CDR2 having the amino acid sequence of SEQ ID NO:38
LC-CDR3 having the amino acid sequence of SEQ ID NO:39.

10. The agent for use, the use or the method according to claim 7 or claim 8, wherein the antibody or antigen-binding fragment comprises:
(i) a heavy chain variable (VH) region incorporating the following CDRs:
HC-CDR1 having the amino acid sequence of SEQ ID NO:40
HC-CDR2 having the amino acid sequence of SEQ ID NO:41
HC-CDR3 having the amino acid sequence of SEQ ID NO:42; and
(ii) a light chain variable (VL) region incorporating the following CDRs:
LC-CDR1 having the amino acid sequence of SEQ ID NO:43
LC-CDR2 having the amino acid sequence of SEQ ID NO:44
LC-CDR3 having the amino acid sequence of SEQ ID NO:45.

11. The agent for use, the use or the method according to claim 7, wherein the agent is an anti-IL-11Rα antibody antagonist of IL-11-mediated signalling, or an antigen-binding fragment thereof.

12. The agent for use, the use or the method according to claim 7 or claim 11, wherein the antibody or antigen-binding fragment comprises:
(i) a heavy chain variable (VH) region incorporating the following CDRs:
HC-CDR1 having the amino acid sequence of SEQ ID NO:46
HC-CDR2 having the amino acid sequence of SEQ ID NO:47
HC-CDR3 having the amino acid sequence of SEQ ID NO:48; and
(ii) a light chain variable (VL) region incorporating the following CDRs:
LC-CDR1 having the amino acid sequence of SEQ ID NO:49
LC-CDR2 having the amino acid sequence of SEQ ID NO:50
LC-CDR3 having the amino acid sequence of SEQ ID NO:51.

13. The agent for use, the use or the method according to claim 5 or claim 6, wherein the agent is a decoy receptor.

14. The agent for use, the use or the method according to claim 13, wherein the agent is a decoy receptor for IL-11.

15. The agent for use, the use or the method according to claim 14, wherein the decoy receptor for IL-11 comprises: (i) an amino acid sequence corresponding to the cytokine binding module of gp130 and (ii) an amino acid sequence corresponding to the cytokine binding module of IL-11Rα.

16. The agent for use, the use or the method according to claim 5 or claim 6, wherein the agent is an IL-11 mutein.

17. The agent for use, the use or the method according to claim 16, wherein the IL-11 mutein is W147A.

18. The agent for use according to claim 1, the use according to claim 2, or the method according to claim 3, wherein the agent is capable of preventing or reducing the expression of interleukin 11 (IL-11) or a receptor for interleukin 11 (1L-11R).

19. The agent for use, the use, or the method according to claim 18, wherein the agent is an oligonucleotide or a small molecule.

20. The agent for use, the use or the method according to claim 19, wherein the agent is an antisense oligonucleotide capable of preventing or reducing the expression of IL-11.

21. The agent for use, the use or the method according to claim 20, wherein the antisense oligonucleotide capable of preventing or reducing the expression of IL-11 is siRNA targeted to IL11 comprising the sequence of SEQ ID NO:12, 13, 14 or 15.

22. The agent for use, the use or the method according to claim 19, wherein the agent is an antisense oligonucleotide capable of preventing or reducing the expression of IL-11Rα.

23. The agent for use, the use or the method according to claim 22, wherein the antisense oligonucleotide capable of preventing or reducing the expression of IL-11Rα is siRNA targeted to IL11RA comprising the sequence of SEQ ID NO:16, 17, 18 or 19.

24. The agent for use, the use or the method according to any one of claims 4 to 23, wherein the interleukin 11 receptor is or comprises IL-11Rα.

25. The agent for use according to any one of claims 1, or 4 to 24, the use according to any one of claims 2, or 4 to 24, or the method according to any one of claims 3 to 24, wherein the method comprises administering the agent to a subject in which expression of interleukin 11 (1L-11) or a receptor for IL-11 (1L-11R) is upregulated.

26. The agent for use according to any one of claims 1, or 4 to 25, the use according to any one of claims 2, or 4 to 25, or the method according to any one of claims 3 to 25, wherein the method comprises administering the agent to a subject in expression of interleukin 11 (1L-11) or a receptor for interleukin 11 (1L-11R) has been determined to be upregulated.

27. The agent for use according to any one of claims 1, or 4 to 26, the use according to any one of claims 2, or 4 to 26, or the method according to any one of claims 3 to 26, wherein the method comprises determining whether expression of interleukin 11 (1L-11) or a receptor for IL-11 (1L-11R) is upregulated in the subject and administering the agent to a subject in which expression of interleukin 11 (1L-11) or a receptor for IL-11 (1L-11R) is upregulated.
